(19) United States
(12) Patent Application Publication
(10) Pub. No.: US 2025/0177367 A1
(43) Pub. Date: Jun. 5, 2025

(54) COMBINATION THERAPIES FOR MODULATION OF LIPID PRODUCTION

(71) Applicant: Ohio State Innovation Foundation, Columbus, OH (US)

(72) Inventors: Deliang GUO, Columbus, OH (US); Chunming CHENG, Columbus, OH (US); Yaogang ZHONG, Columbus, OH (US)

(21) Appl. No.: 18/845,119

(22) PCT Filed: Mar. 9, 2023
(86) PCT No.: PCT/US2023/014883

§ 371 Date: Sep. 9, 2024

Related U.S. Application Data
(60) Provisional application No. 63/318,140, filed on Mar. 9, 2022.
(60) Provisional application No. 63/329,936, filed on Apr. 12, 2022.
(60) Provisional application No. 63/333,784, filed on Apr. 22, 2022.
(60) Provisional application No. 63/338,595, filed on May 5, 2022.

(51) Int. Cl. A61K 31/454; A61K 31/167; A61P 35/00

(52) U.S. Cl. CPC A61K 31/454; A61K 31/167; A61P 35/00

(57) ABSTRACT

The invention is directed to compositions and methods of inhibiting lipogenesis lipid supplies from internal and external sources in a subject. In some embodiments, the invention is directed to compositions and methods for treating cancer, including glioblastoma, by inhibiting lipogenesis lipid supplies in a subject.

### RELATED APPLICATIONS

This application claims the benefit of U.S. Provisional Applications 63/318,140, filed Mar. 9, 2022, 63/329,936, filed Apr. 12, 2022, 63/333,784, filed Apr. 22, 2022, and 63/338,595, filed May 5, 2022, each of which is hereby incorporated in its entirety.

### STATEMENT REGARDING FEDERALLY SPONSORED RESEARCH

This invention was made with Government Support under Grant/Contract Numbers R01 NS112935, R01 CA240726, R01 CA227874, and R01 NS104332 awarded by the National Institutes of Health. The Government has certain rights in the invention.

### FIELD

The present disclosure is directed to compositions and methods of inhibiting lipid supplies in a subject. In some embodiments, the disclosure is directed to compositions and methods for treating a cancer.

### BACKGROUND

Lipids form the basic structure of the plasma membrane and of many cellular organelle membranes. As such, sufficient lipid supply is a precondition for cell growth and proliferation. Under physiological conditions, lipid levels are mainly regulated by sterol regulatory element-binding proteins (SREBPs), a family of transcription factors that regulate numerous cellular processes. SREBP-1 is highly activated in several malignancies including glioblastoma (GBM), liver, breast, and colorectal cancers but the specific mechanisms of activation and lipid metabolism remain elusive. Since rapidly growing and dividing cells (e.g., cancer cells) demand high amounts of fatty acids (FAs) for phospholipid and membrane biogenesis, therapies that reduce fatty acid availability represent a useful modality of cancer treatment. In addition, tumor cells contain large amounts of lipid droplets (LDs), which together with lipoproteins, such as LDL, HDL and VLDL, serve as important sources of fatty acids (FAs) and cholesterol to support tumor growth. LDs and lipoproteins are hydrolyzed in the lysosomes to release their stored FAs and cholesterol to support tumor growth.

Thus, there is a need for improved compositions and methods for inhibiting all lipid sources to treat cancer by concurrently suppressing lipogenesis and lipid release from LDs and lipoproteins in a subject. There is a need for improved compositions and methods for treating cancer, including solid tumors and hard-to-treat cancers. There is a need for improved compositions and methods for treating glioblastoma and other forms of brain cancers. There is a need for increasing the number of reactive oxygen species in a subject. There is a need for controllably imparting mitochondrial damage in a subject.

### BRIEF DESCRIPTION OF FIGURES

The accompanying figures, which are incorporated in and constitute a part of this specification, illustrate several aspects described below.

FIG. TA shows the glutamate, ammonia, and lactate levels in H1299 or U87 cell culture media measured with the Bioprofile 100 Plus Analyzer (mean±SD; n=3). H1299 or U87 cells were cultured in RPMI 1640 or DMEM medium supplemented with 5% FBS for 24 hr. Cells were then washed with PBS once and placed in fresh serum-free RPMI1640 or DMEM medium with or without glutamine (4 mM) or glucose (5 mM) for 12 hr. before measurement. Cell culture conditions prior to treatment for subsequent panels are the same.

FIG. 1B shows the western blot analysis of whole lysates from H1299 or U87 cells cultured in serum-free medium with or without the presence of glucose (5 nM), glutamine (4 mM), glutamate (4 mM), lactate (10 mM) or NH₄Cl (4 mM) for 12 hr.

FIG. 1C show the western blot analysis of whole lysates from H1299 or U87 cells after NH₄Cl stimulation at the indicated doses for 12 hr. in the presence of glucose (5 mM) under serum-free culture conditions.

FIG. 1D shows the western blot analysis of whole lysates from H1299 or U87 cells over time after 4 mM NH₄Cl stimulation in the presence of glucose (5 mM) under serum-free culture conditions.

FIG. 1E shows the representative IF images of anti-SREBP-1 staining (red) in H1299 or U87 cells with or without NH₄Cl (4 mM) stimulation for 12 hr. in the presence of glucose (Gluc, 5 mM) under serum-free medium. Nuclei were stained with DAPI (blue). Scale bars, 10 m. The nuclear intensity of SREBP-1 was quantified over 30 cells by ImageJ (mean±SEM) and shown below. Significance was determined by unpaired Student's t test. *p<0.05, ^{s}p<0.001, #p<0.0001; N.S. not significant.

FIG. 2A shows the western blot analysis of paired tumor (T) vs. adjacent normal (N) lung tissues from individuals with adenocarcinoma (Adeno), squamous cell carcinoma (Squamous) and large cell carcinoma (Large) lung cancer.

FIG. 2B shows the representative immunohistochemistry (IHC) images of anti-GLS and -SREBP-1 staining in tumor vs. adjacent normal lung tissues from individuals with adenocarcinoma or squamous lung cancer. Scale bars, 50 μm.

FIG. 2C shows the ammonia levels in paired human lung tumors vs. adjacent normal lung tissues. Significance was determined by unpaired Student's t test; *p<0.05.

FIG. 2D shows the representative IHC images of anti-GLS and -SREBP-1 staining from a lung cancer tissue microarray (TMA, n=99) that contains 48 adenocarcinoma, 43 squamous, 5 large cell and 3 small cell tumor and 50 paired adjacent normal lung tissues. Scale bars, 100 m.

FIG. 2E shows the levels of GLS expression and SREBP-1 staining from FIG. 2D were quantified by ImageJ and shown by H score. Red lines in the graphs show mean±SEM. Data were analyzed by using one-way ANOVA followed by comparisons with normal control with Dunnett's multiple comparisons adjustment. *p<0.001, **p<0.0001.

FIG. 2F shows the correlation between GLS expression and SREBP-1 levels in tissues from lung cancer TMA shown in FIG. 2C. Correlation coefficient (R) and significance were determined by the Pearson correlation test. p<0.0001.

FIG. 2G shows the representative IHC images of anti-GLS and anti-SREBP-1 staining in tumor tissues from individuals with GBM. Scale bars, 100 μm.

FIG. 2H shows the representative IHC images of anti-GLS and anti-SREBP-1 staining in different types of glioma in a glioma TMA (n=91) that contains 12 A2, 8 AA, 45 GBM, 16 O2 and 10 AO. Scale bars, 100 μm.

FIG. 2I shows the GLS expression and SREBP-1 staining from FIGS. 4G and 4H in TMA were quantified by ImageJ and H score. Red lines in the graphs show mean z SEM. A2, astrocytoma grade II; AA, anaplastic astrocytoma, grade III. GBM, glioblastoma, grade IV; 02, oligodendroglioma, grade 11 and AO anaplastic oligodendroglioma, grade 111.

FIG. 2J shows the correlation between GLS expression and SREBP-1 staining in glioma TMA tissues shown in FIGS. 2H and 2I. The correlation co-efficiency and significance were determined by Pearson's correlation test. p<0.0001.

FIG. 2K shows the Kaplan-Meier curves of the overall survival of individuals with GBM (n=45) from the TMA (FIG. 2H), separated based on the quantification of GLS expression (mean=199.78) or SREBP-1 levels (mean=200.02) (FIG. 2I). Significance was determined by the Log-rank test. p=0.0042 for GLS and p=0.0001 for SREBP-1 comparison.

FIG. 3A shows the effects of GFP-SCAP wild-type or mutant D428A compared to GFP control on SREBP-1 and -2 cleavage in H1299 cells as analyzed by westem blot.

FIG. 3B shows the effects of GFP-SCAP wild-type or mutant D428A on lung tumor growth as analyzed in mice (1×106 cells/mouse) by bioluminescence imaging at day 50 after implantation via tail vein injection.

FIG. 3C shows the tumor growth rate from day 7 to 50 was quantified by bioluminescence imaging (mean±SEM, n=5).

FIG. 3D shows the representative gross images (left panels) and lung sections (right panels) of mouse lungs after hematoxylin and eosin (H&E) staining (middle panels; Scale bars, 2 mm), and of IHC staining of SREBP-1 in tumor tissues (right panels; scale bars, 50 m) from the different groups shown in at day 50 after implantation.

FIG. 3E shows the percentage of tumor nodules occupied per total lung area (upper panel) and the intensity of SREBP-1 staining (lower panel) were quantified by ImageJ (mean±SEM, n=5).

FIG. 3F shows the effects of GFP-SCAP wild-type or mutant D428A compared to GFP control on SREBP-1 and -2 cleavage in primary GBM30 cells analyzed by western blot.

FIG. 3G shows the effects of GFP-SCAP wild-type or mutant D428A on intracranial tumor growth as analyzed in mice (3.5×103 cells/mouse) by magnetic resonance imaging (MRI) (yellow circles). The white arrows indicate the injection sites.

FIG. 3H shows that brain sections were stained with H&E (left panels; scale bars, 1 mm), and IHC for SREBP-1 (right panels, scale bars, 50 in).

FIG. 3I shows that nuclear SREBP-1 staining in tumor tissues was quantified by ImageJ.

FIG. 3J shows the mouse survival was assessed by Kaplan-Meier plot (n=7/group). Significance was determined by one-way ANOVA (FIGS. 3E, 3G, and 3I) or two-way ANOVA (FIG. 3C) with Dunnett's multiple comparisons adjustment. Significance in (FIG. 3J) was determined by Log-rank test. *p<0.05; **p<0.001; ***p<0.0001. N.S. not significant.

FIG. 4A shows the relative levels of ammonia, glutamate, and α-KG in H1299 cells measured with the appropriate assay kit after treating cells with/without GPNA or CB-839. The results are presented as mean±SEM (n=3). H1299 or U87 cells were cultured in RMPI 1640 or DMEM medium supplemented with 5% FBS for 24 hr. Cells then were washed with PBS once and treated with/without GPNA (5 mM) or CB-839 (100 nM) in the presence of glutamine (4 mM) and glucose (5 mM) under the fresh serum-free RMPI 1640 or DMEM medium for 12 hr before analysis. Cell culture conditions prior to treatment are the same for the subsequent panels unless otherwise stated.

FIG. 4B shows the western blot analysis of primary GBM30 cells treated with/without CB-839 (200 nM) for 12 hr in the absence or presence of glutamine, glutamate or NH₄Cl (all 4 mM) under serum-free culture conditions.

FIG. 4C shows the ammonia measurement of tumor tissues (n=6) from H1299 cells (4×106 implantation/mouse)-derived xenograft mouse model treated with vehicle (control, Ctrl) or CB-839 (30 mg/kg mouse weight, i.p. injection, twice per day for 3 days) with anammonia assay kit following the manufacturer's instructions. The treatment started when the tumor size reached 200 mm³. Middle panel shows representative IHC images of SREBP-1 in tumor tissues. Scale bars, 50 m. The expression levels were quantified by using ImageJ to analyze 4 images per tumor and 3 tumors in each group.

FIG. 5A shows the representative IHC images of anti-GLS and -SREBP-1 staining in tumor vs. adjacent normal tissues from individuals with adenocarcinoma (Adeno) or squamous lung cancer. Scale bars, 50 μm.

FIG. 5B shows the representative IHC images of anti-GLS and anti-SREBP-1 staining from lung cancer TMA.

FIG. 5C shows the representative images of different levels of anti-GLS or anti-SREBP-1 staining and scoring.

FIG. 5D shows the comparison of GLS expression and SREBP-1 levels in 50 paired tumors vs. adjacent normal lung tissues from the lung cancer TMA based on H score. Significance was determined by an unpaired Student's t test. All p<0.0001.

FIG. 5E shows the genetic inhibition of GLS or SREBP-1 dramatically suppressed lung tumor growth in vivo. NSCLC H1299 cells were infected with shGLS- or shSREBP-1-expressing lentivirus for 48 hr and then were implanted (2×106 cells/mouse) into the flank of nude mice. The tumors were isolated from mice at 53 days post-implantation and were imaged (left panel) and weighed (right panel) for comparison. Data are shown as mean z SEM (n=6). Significance was determined by one-way ANOVA with Dunnett's multiple comparisons adjustment. *p<0.002.

FIG. 5F shows the representative IHC images of anti-GLS, anti-SREBP-1, anti-ASPG and anti-SDS staining in tumor tissues from patients with GBM. Scale bars, 50 μm.

FIG. 5G shows the representative images of anti-GLS and anti-SREBP-1 staining from glioma TMA.

FIG. 5H shows the representative images of different levels of anti-GLS or anti-SREBP-1 staining and scoring.

FIG. 6A shows the gross and macroscopic images of mouse lungs (a) and H&E staining of lung sections (b) at day 50 after mouse implantation with H1299 cells expressing GFP, wild-type (WT) or mutant GFP-SCAP D428A. Framed images in red were presented in FIG. 3D as representatives. Scale bars, 2 mm.

FIG. 6B shows the number of nodules on mice lung sections was quantified by Image-J (b, lower panel). Data are shown as mean±SEM (n=5). Significance was determined by one-way ANOVA with Dunnett's multiple comparisons adjustment. **p<0.001; ***p<0.0001.

FIG. 6C shows the MRI scans of mouse brain at day 12 after implantation of GBM30 cells stably transfected with GFP, wild-type or mutant (D428A) GFP-SCAP (3.5×103 cells/mouse). Yellow circles indicate tumor location. White arrows indicate injection site. Scatter blot shows tumor volume from MRI scans quantified from the outlined region-of-interest (ROIs) (right panel). Significance was determined by unpaired Student's t test. *p<0.05.

FIG. 6D shows the H&E staining of mouse brain sections excised at day 17 after implantation of GBM30 cells as described in FIG. 6C. Rectangle-framed images were used in FIG. 3H as representatives. Scale bars, 1 mm.

FIG. 7 shows the relative growth of GBM cell line (U373) in response to different antipsychotic drug treatment and identifying pimozide is the most potent drug to inhibit GBM cell proliferation. 3×10⁴ U373 cells were cultured in 12-wells plate in full DMEM medium with 4.5 g/L glucose (25 mM) and 5% FBS for 24 hour, and then washed with PBS twice and replaced with fresh DMEM medium containing 1% FBS and 5 mM glucose, 4 mM glutamine and 1 mM pyruvate. Antipsychotic drugs were then added into each well at indicated concentrations (0-100 μM) and live cells were counted by trypan blue staining after 48 hour treatment and normalized with control cell numbers with DMSO treatment. PMZ, pimozide; HAL, Haloperidol; IMI, Imipramine; CLO, Clozapine; OLA, Olanzapine; PER, Perphenzaine; PRO, Promazine; SUL, sulpride; UA, U18666A. DMSO was used as a buffer solution for all drugs.

FIG. 8A shows the GBM30 (patient-derived primary GBM cells) is mesenchymal cells with a mutated EGFRvIII (constitutive EGFR mutation that lacks EGFR exons 2-7) and wild-type IDH. GBM30 was cultured in DMEM/F12 medium supplemented with B-27 (1×), heparin (2 μg/mL), EGF (20 ng/mL), and fibroblast growth factor (FGF, 20 ng/mL) in Geltrex matrix coated 6 cm dish with glass bottom, and U251 cells were cultured in full DMEM medium containing 5% FBS and 25 mM glucose in 6 cm dish with glass bottom for 24 hr. After PBS wash once and replaced with fresh DMEM medium containing 1% FBS and 5 mM glucose, 4 mM glutamine and 1 mM pyruvate after PBS wash twice, cells were then treated with/without pimozide (3 μM) for 24 hr before staining with BODIPY 493/503 (green)/Lysotracker (red)/Hoechst 33342 (blue) and observing by confocal microscopy. LD numbers stained with BODIPY were dramatically diminished after 1% FBS culture for 24 hr in comparing with pre-treatment condition in the absence of pimozide. In contrast, pimozide treatment blocked LDs diminish, which were largely accumulated in Lysotracker-stained lysosomes.

FIG. 8B shows the representative fluorescence imaging of lysosome stained with lysotracker (Red) (n=30 images in total) in GIBM30 and U251 cells were treated with/without pimozide (3 μM) as the same cultured conditions as in (A) for 24 hr. Lysosome sizes were dramatically increased after pimozide treatment,

FIG. 8C shows the determination of lysosomal activity by DQ-green BSA. GBM30 and U251 cells were treated with/without pimozide (3 μM) for 24 hr in 1% FBS, 5 NM glucose, 4 mM glutanine, 1 mM pyruvate medium, washed with PBS twice and then incubated with 10 μg/mL DQ-green BSA in fresh DMEM medium containing 1% FBS and 1% NEAA, 1% GlutaMax, and 1% HEPES for 6 hr before observation by confocal microscopy. The cell nucleus was stained with Hoechst 33342 (blue). In control cells with DMSO treatment, DQ-Green-BSA was endocytosed and delivered to the lysosomes, where DQ-Green-BSA was degraded by active acidic lysosome hydrolase that de-quenched the fluorescence of the dye, thereby showing the bright green spots in the lysosomes. In contrast, when lysosomal activity was inhibited. DQ-Green-BSA failed to degrade, resulting in fluorescence of the dye remaining in quenched (PMZ treatment). The dramatically reduced green fluorescence in pimozide treatment compared with control DMSO treatment demonstrated lysosomal activity was significantly inhibited by pimozide.

FIG. 8D shows the determination of lysosomal pH with the ratiometric probe LysoSensor Yellow/Blue dextran. GBM30 and U251 cells were treated as panel C for 24 hr and incubated, protected from light, with 1 mg/ml LysoSensor Yellow/Blue dextran for 24 h before observation by confocal microscopy, Yellow fluorescence represents more acidic lysosomal environment (pH˜4.5), and blue fluorescence represents more neutral lysosomal environment (pH˜60). The data clearly showed that pimozide treatment dramatically increased lysosomal pH from acidic to neutral level.

FIG. 9A shows the western blot analysis of whole lysates of GBM30 cultured in DMEM/F12 supplemented with B-27 (1×) serum-free supplements, heparin (2 μg/mL), EGF (20 ng/mL), and fibroblast growth factor (FGF. 20 ng/mL) and U251 cells in DMEM medium containing 1% FBS, 5 mM Glucose, 4 mM Glutamine and 1 mM Pyruvate after treatment with different doses of pimozide for 24 hr. P, precursor of SREBP; N, N-terminus of SREBP-1. C, C-terminus of SREBP-2. The data showed that pimozide dose-dependently activated SREBP-1/2 cleavage and its downstream fatty acid synthesis targets FASN and SCD1 expression.

FIG. 9B shows the real-time PCR examination of gene expression in the fatty acid and cholesterol synthesis pathways in GBM30 and U373 cells under the same culture/treatment condition shown above. The results are shown as mean±SD (n=3). FASN, fatty acid synthase; LDLR, low-density lipoprotein receptor; SCD1, stearoyl-CoA desaturase 1; hMGCS1, 3-Hydroxy-3-Methylglutaryl-CoA Synthase 1; SRFBF1, the gene name of SREBP-1; ACLY, ATP citrate lyase; ACACA, acetyl-CoA carboxylase alpha; HMGCR, 3-hydroxy-3-methylglutaryl-CoA reductase. The data showed that pimozide treatment significantly increased the expression of genes in controlling fatty acid/cholesterol synthesis.

FIG. 9C shows the western blot analysis of membrane extracts (for ASCT2) from GBM30 and U251 cells under the same culture/treatment conditions as in (A). Transferring receptor-1(CD71), an integral membrane protein that mediates the uptake of transferrin-iron complexes, was used as a membrane loading control for ASCT2. The data showed that pimozide treatment dose-dependently increased ASCT2 expression.

FIG. 9D shows the representative confocal immuno-fluorescence images of anti-ASCT2 staining (green) in GBM30 and U251 cells under the same culture conditions as in (A). Nuclei were stained with DAPI (blue). The data showed that pimozide treatment dose-dependently increased ASCT2 expression.

FIG. 9E shows the western blot analysis of whole lysates and membrane protein from GBM30 and U251 cells treated with/without pimozide (5 μM) and cholesterol (3 μg/ml) for 24 hr in same culture condition as panel (A). The data showed that pimozide treatment-elevated ASCT2 expression was suppressed by cholesterol addition, accompanying with the suppression of SREBP-1 activation.

FIG. 9F shows the real-time PCR analysis of SREBP-1 association with ASCT2 gene promoter (also known as SLC1A5 gene) after chromatin-immunoprecipitation (ChIP) by using anti-SREBP-1 antibody. Top panel shows putative SREBP-1 binding site (SRE) in ASCT2 promoter by using xxx promoter analysis. The data showed that SRtEBP-1 binds to ASCT2 gene promoter.

FIG. 9G shows the analysis of SREBP-1 transcriptional activity on ASCT2 gene promoter via promoter-luciferase reporter assay. U373 cells were transfected with pGL3-luc plasmid containing different length of ASCT2 gene promoter with/without SREBP-1 putative binding site for 24 hr in DMEM medium with 5% FBS, and then infected with adenovirus expression null, N-terminal ad-SREBP-1a, -1c or -2 for 24 hr before analysis. The data showed that N-terminal SREBP-1a is the major form to activate ASCT2 gene promoter.

FIG. 9H shows the western blot analysis of ASCT2 expression in GBM cells after overexpression of N-terminal active SREBP-1a, -1c or -2 form. GBM cells were infected with adenovirus expressing null, N-terminal SREBP-1a, -1c or -2 for 48 hr in 5% FBS medium. Cells were then lysed, and total cell lysates and membrane extracts were analyzed by western blot. The data showed that N-terminal SREBP-1a is the major form to activate ASCT2 protein expression.

FIG. 10A shows the western blot analysis of whole lysates or membrane extracts of GBM30 cultured in DMEM/F12 supplemented with B-27 (1×) serum-free supplements, heparin (2 μg/mL), EGF (20 ng/mL), and fibroblast growth factor (FGF, 20 ng/mL), U373 or U251 cells after treatment with/without pimozide (5 μM) in the presence or absence of glutamine (4 mM) or NH₃·H₂O (4 mM) for 24 hr in 1% FBS culturing condition containing 5 mM glucose and 1 mM pyruvate. The data show that glutanine absence completely abolished pimozide-stimulated SREBP-1 activation, FASN/SCD1 and ASCT2 expression, while which were fully restored by ammonia (NH₄⁺) derived from NH₃·H₂O, demonstrating that ammonia released from glutamine is an essential activator for pimozide-mediated SREBP-1/lipogenesis pathway activation and ASCT2 upregulation.

FIG. 10B shows the western blot analysis of whole lysates or membrane extracts of GBM30 cultured in DMEM/F12 supplemented with B-27 (1×) serum-free supplements, heparin (2 μg/mL), EGF (20 ng/mL), and fibroblast growth factor (FGF, 20 ng/mL), U373 or U251 cells after treatment with/without pimozide (5 μM), GPNA (1 nM, ASCT2 inhibitor) in the presence or absence of NH₃·H₂O (4 mM) for 24 hr in 1% FBS culturing condition containing 5 mM glucose. 4 mM glutamine and 1 nM pyruvate. The data show that pharmacologically inhibiting glutamine uptake by GPNA completely abolished pimozide-stimulated SREBP-1 activation, FASN/SCD1 and ASCT2 expression, while which were fully restored by ammonia (NH₄⁺) derived from NH₃·H₂O, further demonstrating that ammonia released from glutamine is an essential activator for pimozide-mediated SREBP-1/lipogenesis pathway activation and ASCT2 upregulation.

FIG. 10C shows the western blot analysis of whole lysates or membrane extracts of GBM30 cultured in DMEM/F12 supplemented with B-27 (1×) serum-free supplements, heparin (2 μg/mL), EGF (20 ng/mL), and fibroblast growth factor (FGF, 20 ng/mL), U373 or U251 cells after treatment with/without pimozide (5 μM) or CB-839 (100 nM, glutaminolysis enzyme GLS inhibitor) (C) in the presence or absence of NH₃·H₂O (4 mM) for 24 hr in 1% FBS culturing condition containing 5 mM glucose, 4 mM glutamine and 1 mM pyruvate. The data show that pharmacologically inhibiting GLS by CB-839 completely abolished pimozide-stimulated SREBP-1 activation, FASN/SCD1 and ASCT2 expression, while which were fully restored by ammonia (NH₄⁺) derived from NH₃·H₂O, further demonstrating that ammonia released from glutamine is an essential activator for pimozide-mediated SREBP-1/lipogenesis pathway activation and ASCT2 upregulation.

FIG. 11A shows the representative confocal fluorescence imaging (n=12 images in total) of U1251 cells stained with MitoTracker (staining mitochondria. Green) and CellROX Deep Red (detect reactive oxygen species, ROS) after treatment with/without pimozide (3 μM), GINA (1 mM), DON (10 μM) or CB-839 (100 nM) alone or combination in 1% FBS, 5 mM Glucose, 4 nM Glutanmine, 1 mM Pyruvate medium for 24 hr. ROS levels were quantified in more than 100 cells (mean±SEM). Sp<0.0001. Scale bar, 10 μm. The data show that the combination of pimozide and glutamine metabolism inhibitors leads to mitochondria fragmentation and dramatic elevation of ROS occurred in mitochondria.

FIG. 11B shows the western blot analysis of whole lysates from GBM30, U373 or U251 cells treated with/without Pimozide (5 μM), GPNA (1 mM), DON (10 μM) or CB-839 (10 nM) for 48 hr as the same conditions as in (A). The data show that the combination of pimozide and glutamine metabolism inhibitors dramatically increased apoptosis markers caspase and PARP cleavage, demonstrating the combination results in marked tumor cell apoptosis.

FIG. 11C shows the proliferation curves of different cancer cells, GBM30 was cultured in DMEM/F12 supplemented with B-27 (1×) serum-free supplements, heparin (2 μg/mL), EGF (20 ng/mL), and fibroblast growth factor (FGF, 20 ng/mL) with/without pimozide (3 μM), GPNA (1 mM), DON (10 μM) or CB-839 (100 nM) for 9 days. U373 and U251 were cultured in medium supplemented with 5% FBS for 24 hr. Cells were then washed with PBS twice and treated in 1% FBS with the presence of 5 mM Glucose, 4 mM Glutamine and 1 mM Pyruvate in fresh serum-free medium with/without pimozide (3 μM), GPNA (1 mM), DON (10 μM) or CB-839 (100 nM) for 4 days (mean±SD, n=3). The data show that the combination of pimozide and glutamine metabolism inhibitors dramatically inhibited tumor cell proliferation.

FIG. 11D shows the representative confocal fluorescence images of U251 cells stained with MitoTracker (staining mnitochondria, Green) and CellR-OX Deep Red (detecting ROS level) after treatment with/without pimozide (3 μM), GPNA (1 mM), DON (10 μM) or CB-839 (100 nM) alone or in combination as in (A) in the presence or absence of GSH (3 mM). The data show that the antioxidant GSH reduced the combination treatment-elevated ROS level in mitochondria and restored mitochondria morphology to the levels similar as control cells without treatment, demonstrating that the combination of pimozide and glutamine metabolism inhibitors induced ROS caused mitochondria fragmentation and damage. Scale bar, 10 μm.

FIG. 11E shows the representative cell micrographs of 5251 after treatment with pimozide (3 μM), GPNA (1 mM). DON (10 μM) or CB-839 (100 nM) alone or in combination as in (A) in the presence or absence of GSH (3 mM) for 48 hr. The data show that the antioxidant GSH dramatically rescued the combination-induced cell death.

FIG. 11F shows the representative confocal fluorescence images of U251 cells stained with MitoTracker (staining mitochondria, Green) and CellROX Deep Red (detecting ROS level) after treatment with/without pimozide (3 μM), GPNA (1 mM). DON (10 μM) or CB-839 (I00 nM) in combination as in (A) in the presence or absence of PA (palmitate, 20 μM) and OA (oleic acid, 20 μM) and cholesterol (3 μg/ml) for 24 hr. The data show that addition of fatty acid and cholesterol dramatically reduced pimozide and glutamine metabolism inhibitor combination-caused ROS and restored mitochondria morphology.

FIG. 11G shows the western blot of GBM30, U5251 and 15373 cells treated with/without pimozide (3 μM), GPNA (1 mM), DON (10 μM) or CB-839 (100 nM) in combination as in (A) in the presence or absence of PA (palmitate, 20 μM) and OA (oleic acid, 20 μM) and cholesterol (3 μg/ML) for 48 hr, Cyto, cytosol; Mito, mitochondria. The data show that addition of fatty acid and cholesterol dramatically reduced pimozide and glutamine metabolism inhibitor combination-induced apoptosis marker cleavage, demonstrating that the combination-caused cell death is triggered by limitation of fatty acid/cholesterol availability.

FIG. 11H shows the micrographs show the growth of U251 treated with pimozide (3 μM) and GPNA (1 mM) as in (A) in the presence or absence of PA (palmitate, 20 μM) and OA (oleic acid, 20 μM) and cholesterol (3 μg/mL). Cells were cultured in 5% FBS full DMEM medium for 24 hr. Cells were then washed with PBS twice and treated in 1% FBS with the presence of 5 mM Glucose, 4 mM Glutamine and 1 mM Pyruvate in fresh serum-free medium for 72 hr. Scale bar, 100 μm.

FIG. 12A shows the pimozide dose-dependently inhibited tumor growth derived by GBM30 cells. Mice were implanted with 3×10⁶ GBM30 cells in flank. When tumor volume reached to 80 mm³, pimozide (15, 30, 60 mg/kg/day) or vehicles were administered to mice via intraperitoneal injection for 14 days. FIG. 12A also shows tumor images after isolation from mice at the last day of treatment.

FIG. 12B shows the fold change of tumor growth curve as normalized With tumor volume prior to the treatment (day 0) (left), and tumor volume (right) was shown in right panel. 60 mg/kg dose was toxic, and mice died after treatment for 6-8 days.

FIG. 12C shows the colony formation assay to examine the combination effects of pimozide and fatostatin in GBM cells. GBM30 were cultured in DMEM/F12 supplemented with B-27 (1×) serum-free supplements, heparin (2 g/mL), EGF (20 ng/mL), and fibroblast growth factor (FGF, 20 ng/mL), U251 and U373 were first cultured in 5% FBS full medium to grow 6 days without treatment to form colony Cells were then washed with PBS twice and treated with/without pimozide (3 μM), Fatostatin (5 μM) in fresh medium containing 1% FBS, 5 mM glucose, 4 mM glutamine, 1 mM pyruvate medium for 8 days, and colony numbers were quantified. The data showed that either pimozide or fatostatin single treatment only slightly reduced colony number, while their combination almost completely eradicated pre-formed colonies.

FIG. 12D shows the combination of pimozide and SREBP fatostatin synergistically inhibited tumor growth in GBM30-derived xenograft model. GBM30 cells were implanted in mice to form tumor same as FIG. 12A. Mice were started treatment when tumor volume reached to ˜80 mm³ by pimozide (15 mg/kg/day) and fatostatin (25 mg/kg/day) alone or combination for 16 days. The tumors were isolated from mice at day 14 post-treatment and imaged. Data are shown as mean±SD (n=6).

FIG. 12E shows the fold change of tumor growth curve as normalized with day 0 tumor volume prior to treatment (left) and tumor volume without normalization. Data are shown as mean±SD(n=6). Significance was determined by one-way ANOVA with Dunnett's multiple comparisons adjustment. *p<0.002. Tumor diameter was measured using vernier caliper and calculated as V=L×S×S/2 (S refers to short diameter, L means long diameter, V is tumor volume). The data showed that combination of pimozide and fatostatin synergistically inhibited tumor growth in GBM30-derived xenograft model.

FIG. 13A shows the colony formation assay to examine the effects of pimozide treatment in GBM cells in the presence and absence of glutamine. GBM30 was cultured in DMEM/F12 supplemented with B-27 (1×) serum-free supplements, heparin (2 μg/mL), EGF (20 ng/mL), and fibroblast growth factor (FGF, 20 ng/mL), and U373, U251 Cells were first cultured in 5% FBS full medium to grow 6 days to form colony. Cells were then washed with PBS twice and treated with pimozide (3 μM) in the presence or absence of glutamine (4 mM) in fresh medium containing 1% FBS and 5 mM glucose, 1 mM pyruvate. The data show that in the absence of glutamine, pimozide almost eradicated pre-formed colonies.

FIG. 13B shows the GBM30 was cultured in DMEM/F12 supplemented with B-27 (1×) serum-free supplements, heparin (2 μg/mL), EGF (20 ng/mL), and fibroblast growth factor (FGF, 20 ng/mL), Proliferation curves of U373 and 13251 cells cultured in medium supplemented with 1% FBS with/without pimozide (3 μM), or glutamine (4 mM) for 4 days (mean±SD, n=3). Cells were cultured in medium supplemented with 5% FBS for 24 hr. Cells were then washed with PBS twice and treated in 1% FBS with the presence of 5 mM glucose, and 1 mM pyruvate in fresh medium. The data show that pimozide kills GBM cells in the absence of glutamine.

FIG. 13C shows the colony formation assay to examine the therapeutic effects of the combination of pimozide with glutamine metabolism inhibitors. Cells were first cultured in 5% FBS full medium to grow 6 days to form colony. Cells were then washed with PBS twice and treated with pimozide (3 μM), GPNA (1 mM), DON (10 μM) or CB-839 (100 nM) alone or in combination in 1% FBS medium containing 5 mM glucose, 4 mM glutamine, 1 mM Pyruvate for 8 days. The data show that the combination eradicated almost all pretreatment-formed colonies.

FIG. 13D shows the proliferation curves of different cancer cells cultured in medium supplemented with 1% FBS with/without pimozide (3 μM), GPNA (1 mM), DON (10 μM) or CB-839 (100 nM) alone or in combination. Cells were first cultured in medium supplemented with 5% FBS for 24 hr. Cells were then washed with PBS twice and treated in 1% FBS with the presence of 5 mM Glucose, and 1 mM Pyruvate for treatment for 4 days. The data show that the combination almost eradicated almost all killed almost all tumor cells.

FIG. 13E shows the combination of glutamine metabolism inhibition with pimozide synergistically inhibit tumor growth in GBM30-derived xenograft model. Mice were implanted with 3×10⁶ GBM30 cells in flank. When tumor volume reached to ˜80 mm³, pimozide (15 mg/kg/day), GPNA (50 mg/kg/day), DON (0.2 mg/kg/day), or CB-839 (25 mg/kg/day) alone or in combination were administered to mice via intraperitoneal injection for 14 days. FIG. 22E also shows the tumor images.

FIG. 13F shows the tumor weight after isolation from mice at the last day of treatment.

FIG. 13G shows the fold change of tumor growth curve as normalized with tumor volume prior to the treatment (day 0). Data are shown as mean±SEM (n=6). Significance was determined by one-way ANOVA with Dunnett's multiple comparisons adjustment. *p<0.002. The data show that the combination synergistically inhibited tumor growth.

FIG. 14 shows the inhibition of glutamine utilization sensitizes GBM cells to other antipsychotic drug treatment. Micrographs show the growth of U251 cells treated with/without pimozide (2, 3 μM), perphenazine (3, 5 μM), GPNA (1 mM), or CB-839 (100 nM). Cells were cultured in 5% FBS full medium for 24 hr. Cells were then washed with PBS twice and treated in 1% FBS with the presence of 5 mM Glucose, 4 mM Glutamine and 1 mM Pyruvate in fresh serum-free medium for 48 hr. Scale bar, 100 μm.

FIG. 15 shows the inhibition of glutamine utilization sensitizes breast cancer cells to antipsychotic drug treatment. Micrographs show the growth of breast cancer cell line (MDA-MB-231), liver cancer cell line (Huh7) and lung cancer cell line (H1299) treated with/without pimozide (2, 3 μM), perphenazine (3, 5 μM), GPNA (1 mM), or CB-839 (100 nM). Cells were cultured in 5% FBS full DMEM/RPMI1640 medium for 24 hr. Cells were then washed with PBS twice and treated in 1% FBS with the presence of 5 mM Glucose, 4 mM Glutamine and 1 mM Pyruvate in fresh serum-free medium for 48 hr. Scale bar, 100 μm.

FIG. 16 shows the inhibition of glutamine utilization sensitizes lung cancer cells to antipsychotic drug treatment. Micrographs show the growth of lung cancer cell line (H1299), liver cancer cell line (Huh7) and lung cancer cell line (H1299) treated with/without pimozide (2, 3 μM), perphenazine (3, 5 μM), GPNA (1 mM), or CB-839 (100 nM). Cells were cultured in 5% FBS full DMEM/RPMI1640 medium for 24 hr. Cells were then washed with PBS twice and treated in 1% FBS with the presence of 5 mM Glucose, 4 mM Glutamine and 1 mM Pyruvate in fresh serum-free medium for 48 hr. Scale bar, 100 μm.

FIG. 17 shows the shows the inhibition of glutamine utilization sensitizes lung cancer cells to antipsychotic drug treatment. Micrographs show the growth of lung cancer cell line (H1299) treated with/without pimozide (2, 3 μM), perphenazine (3, 5 μM), GPNA (1 mM), or CB-839 (100 nM). Cells were cultured in 5% FBS full DMEM/RPMI1640 medium for 24 hr. Cells were then washed with PBS twice and treated in 1% FBS with the presence of 5 mM Glucose, 4 mM Glutamine and 1 mM Pyruvate in fresh serum-free medium for 48 hr. Scale bar, 100 μm.

FIG. 18A shows the representative MRI imaging shows the effects of glutamine transporter ASCT2 inhibitor (GPNA, 50 mg/kg/daily, i.p.), glutaminase inhibitor (CB-839, 20 mg/kg/daily, i.p.), and SREBP-1 inhibitor (Fatostatin, 25 mg/kg/daily, i.p.) combined with/without Pimozide (PMZ, 25 mg/kg/daily, i.p.) compared to vehicle control in primary GBM30 cells-derived intracranial GBM model after 12 days treatment. Tumor in mice brain was highlighted by red circle. 5×10⁴ cells were implanted into mice brain. Drug treatment started after 7 days implantation.

FIG. 18B shows the mouse survival was assessed by Kaplan-Meier plot (n=7/group) for the mice treatment same as above. Significance was determined by Log-rank test. The statistical significance between Pimozide (PMZ)/fatostatin (Fato) combination drug treatment. *p<0.01. All signal drug treatment vs. control are no significance.

FIG. 18C shows the mouse survival was assessed by Kaplan-Meier plot (n=7/group) for the mice treatment same as above. Significance was determined by Log-rank test. The statistical significance between PMZ/GPNA, PMZ/CB-839 vs. vehicle and signal drug treatment is: *p<0.01. All signal drug treatment vs. control are no significance.

FIG. 19A shows the relative cell viability of patient-derived cell GBM30 (6 days) and U251 (3 days) after different antipsychotic drug treatment at the indicated doses. (PMZ, pimozide; FLU, fluoxetine; HAL, haloperidol; IMI, imipramine; CLO, clozapine; OLA, olanzapine; PER, perphenazine; PRO, promazine; SUL, sulpiride).

FIG. 19B shows the representative micrographs showing the effects of PMZ treatment on normal human astrocytes (NHA, 6 days), GBM30 (6 days) and U251 (3 days); Scale bar, 50 μm.

FIG. 19C shows the quantification of death percentage upon PMZ treatment as above. Statistical significance was analyzed by one-way ANOVA.

FIG. 20 shows the representative fluorescence imaging of the distribution of octane-amine linked Pacific blue (20 μM) in the plasma membrane of U251 cells after 24 hr. Lysosomes and mitochondria were co-stained with LysoTracker (red) and MitoTracker (green).

FIG. 21A shows the representative fluorescence imaging of LDs and lysosomes in GBM30 cells stained with BODPIY493/503 (green) and Lysotracker (red) after treatment with/without pimozide (PMZ) for 24 hr in 5% FBS or 1% FBS culturing conditions.

FIG. 21B shows the representative fluorescence imaging of LDL in GBM30 cells after treatment with/without pimozide (PMZ, 5 μM) for 24 hr and then supplemented with BODIPY-labeled LDL (green) for 4 hr, followed by replacing with fresh medium without containing BODIPY-LDL for 16 hr. Scale bar, 10 μm.

FIG. 22A shows the effects of pimozide (PMZ, 3 μM) treatment with/without combination with ASCT2 (GPNA, G, 0.5 mM), GLS (CB-839, C, 100 nM) or a general glutamine metabolism (6-diazo-5-oxo-I-norleucine, DON, D, 10 μM) inhibitors for 8 days in GBM30 cell-derived colonies grown for 6 days prior to treatment.

FIG. 22B shows the colonies were quantified by ImageJ at day 8 after treatment (mean±s.d., n=3).

FIG. 23 shows the representative fluorescence imaging of mitochondria in GBM U251 cells after treatment with/without pimozide (3 μM), GPNA (0.5 mM), DON (10 μM) and CB-839 (100 nM) for 24 hr. Scale bar, 10 μm.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers, or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compositions described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomers. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques el al., Enantiomers, Racemates, and Resolutions, Wiley Interscience, New York, 1981; Wilen et al., Tetrahedron 33: 2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or another labile atom. By way of example:

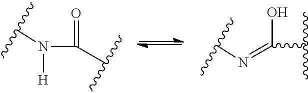

The prevalence of one tautomeric form over another will depend both on the specific chemical compound as well as its local chemical environment. Unless specified to the contrary, the depiction of one tautomeric form is inclusive of all possible tautomeric forms.

Compounds disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acid such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Unless specified explicitly as the free base or free acid, reference to compound that is capable of forming a salt embraces both pharmaceutically acceptable salts and the freebase/acid.

In one aspect, disclosed herein are methods and compositions for treating cancers, diseases related to neoplastic cellular growth, or diseases associated with increased lipid utilization. In one aspect, disclosed herein is a method of treating cancer in a patient in need thereof, comprising administering to the patient at least one ammonia suppressing agent alone or in combination with one or more lipid metabolism inhibitors, or related agents thereof. In one aspect, disclosed herein are methods and compositions for targeting and/or inhibiting multiple pathways along the lipogenesis pathways. In some implementations, the ammonia suppressing agent(s) and lipid metabolism inhibitor(s) synergistically combine to enhance therapeutic potency. In some embodiments, the one or more lipid metabolism inhibitors includes at least one inhibitor suppressing the utilization/release of lipids from extracellular uptake (LDL) and internal storage (lipid droplets, LDs). In some embodiments, the compositions and methods include one or more ammonia suppressing agents and/or one or more inhibitors suppressing the utilization/release of lipids from extracellular uptake (LDL) and internal storage (lipid droplets, LDs).

In some embodiments, the inhibitor suppressing the utilization/release of lipids from extracellular uptake and internal storage includes a SREBP inhibitor, fatty acid synthesis inhibitor, cholesterol synthesis pathway inhibitor, lysosome dysregulating agent, or combination thereof.

In some embodiments, the method includes administering at least one ammonia suppressing agent and at least one lysosome dysregulating agent. In some embodiments, the method includes administering at least one ammonia suppressing agent and at least one SREBP inhibitor. In some embodiments, the method includes administering at least one ammonia suppressing agent and at least one fatty acid synthesis inhibitor. In some embodiments, the method includes administering at least one ammonia suppressing agent and at least one cholesterol synthesis pathway inhibitor.

In some embodiments, the method includes administering at least one ammonia suppressing agent, at least one lysosome dysregulating agent, and at least one SREBP inhibitor. In some embodiments, the method includes administering at least one ammonia suppressing agent, at least one lysosome dysregulating agent, and at least one fatty acid synthesis inhibitor. In some embodiments, the method includes administering at least one ammonia suppressing agent, at least one lysosome dysregulating agent, and at least one cholesterol synthesis pathway inhibitor.

In some embodiments, the lysosome dysregulating agent increases lysosomal pH.

In some embodiments, the lysosome dysregulating agent includes an antibiotic, an antipsychotic, an antimalarial, an amebicide, a chemical chaperone, an antidepressant, an antiparasitic, a mucolytic agent, an isoflavone, a monosaccharide analog, a calcium channel agonist or activator, a potassium channel agonist or activator, a micropeptide, an antiepileptic, an immunosuppressant, an antiviral/anticancer inhibitor, a cathepsin inhibitor, a proteinase inhibitor or peptidase inhibitor, aluminum oxide compound or derivative thereof, a kinase inhibitor, a fatty acid synthesis inhibitor, a cholesterol synthesis inhibitor, a serotonin or dopamine inhibitor, an exosome-related inhibitor, a galactosidase inhibitor, a heat shock protein (HSP) inhibitor, a piperidine, a bone disease-related inhibitor, or combinations thereof.

In some embodiments, the antibiotic includes bafilomycin A, concanamycin, salicylihalamide, oximidine, or combinations thereof. In some embodiments, the antipsychotic includes pimozide, haloperidol, clozapine, olanzapine, perphenazine, promazine, sulpiride, penfluridol, olanzapine, chlorpromazine, or combinations thereof. In some embodiments, the antimalarial includes chloroquine, hydroxychloroquine, or combinations thereof. In some embodiments, the chemical chaperone includes migalasatat, N-octyl-β-valienamine, NCGC607, or combinations thereof. In some embodiments, the antidepressant includes fluoxetine. In some embodiments, the antiparasitic includes pyrimethamine.

In some embodiments, the mucolytic agent includes N-acetylcysteine, ambroxol, monensin, or combinations thereof. In some embodiments, the isoflavone includes genistein, 3,4,7-trihydroxyisoflavone, or a combination thereof. In some embodiments, the monosaccharide analog includes afegostat. In some embodiments, the calcium channel agonist or activator includes ML-SA1, MK6-83, or a combination thereof. In some embodiments, the potassium channel agonist or activator includes ICA-069673. In some embodiments, the micropeptide includes humanin, SD1002, or a combination thereof. In some embodiments, the antiepileptic includes retigabine. In some embodiments, the immunosuppressant includes rapamycin, sirolimus, P140, or combinations thereof.

In some embodiments, the antiviral/anticancer inhibitor includes apilimod, BRD 1240, saliphenylhalamide, or combinations thereof. In some embodiments, the cathepsin inhibitor includes R05461111, odanacatib, CA030, CA-074, CLIK-164, CLIK-181, CLIK-195, SB-357114, L-006235, LHVS (also referred to as Mu-Leu-HphVSPh), or combinations thereof. In some embodiments, the proteinase or peptidase inhibitor includes pepstatin A, α1-antichymotrypsin, CLIK-148, or combinations thereof. In some embodiments, the aluminum oxide compound includes SD1003 or derivatives thereof. In some embodiments, the kinase inhibitor includes Ly294002, YM-201636, YM-201636, or combinations thereof. In some embodiments, the fatty acid synthesis inhibitor includes eliglustat, ibiglustat, lucrerastat, or combinations thereof.

In some embodiments, the cholesterol synthesis inhibitor includes U18666A, lonafarnib, tipifamib, or combinations thereof. In some embodiments, the serotonin or dopamine inhibitor includes SF-22. In some embodiments, the exosome inhibitor includes GW4869. In some embodiments, the galactosidase inhibitor includes deoxygalactonojirimycin. In some embodiments, the HSP inhibitor includes VER-155008. In some embodiments, the piperidine includes miglustat. In some embodiments, the bone disease-related inhibitor includes SB-242784, FR167356, or a combination thereof. In some embodiments, the lysosome dysregulating agent includes α-logeline, 5N,6S-(N′-butyliminomethylidene)-6-thio-1-deoxygalactonojirimycin, PADK, or combinations thereof.

In some embodiments, the lysosome dysregulating agent is P140 peptide, a synthetic peptide currently in Phase III trials for lupus. In some implementations, the lysosome dysregulating agent has the formula:

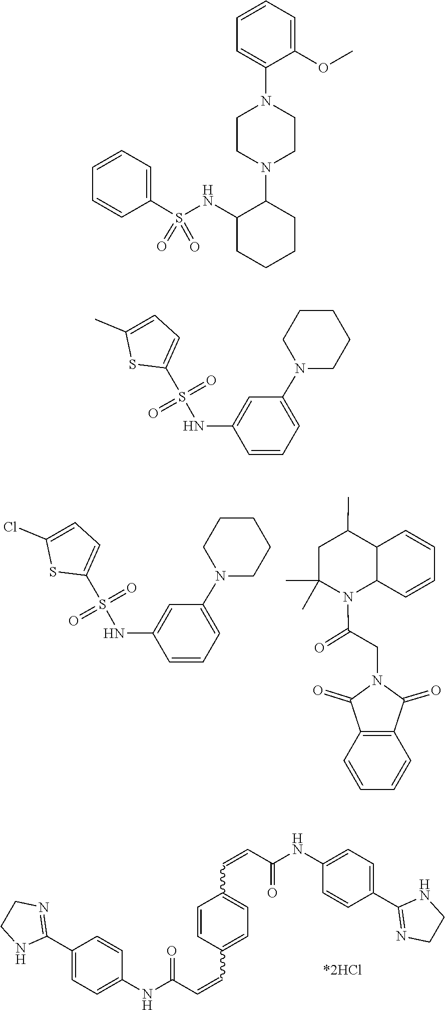

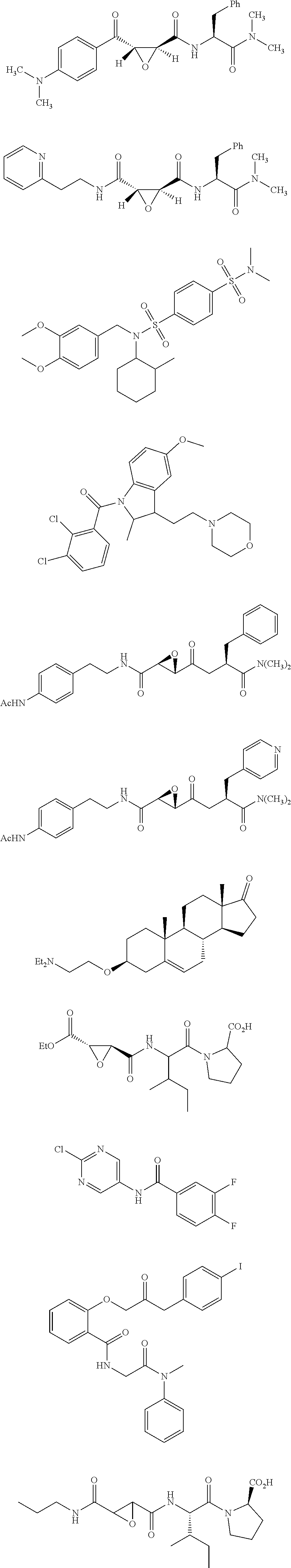

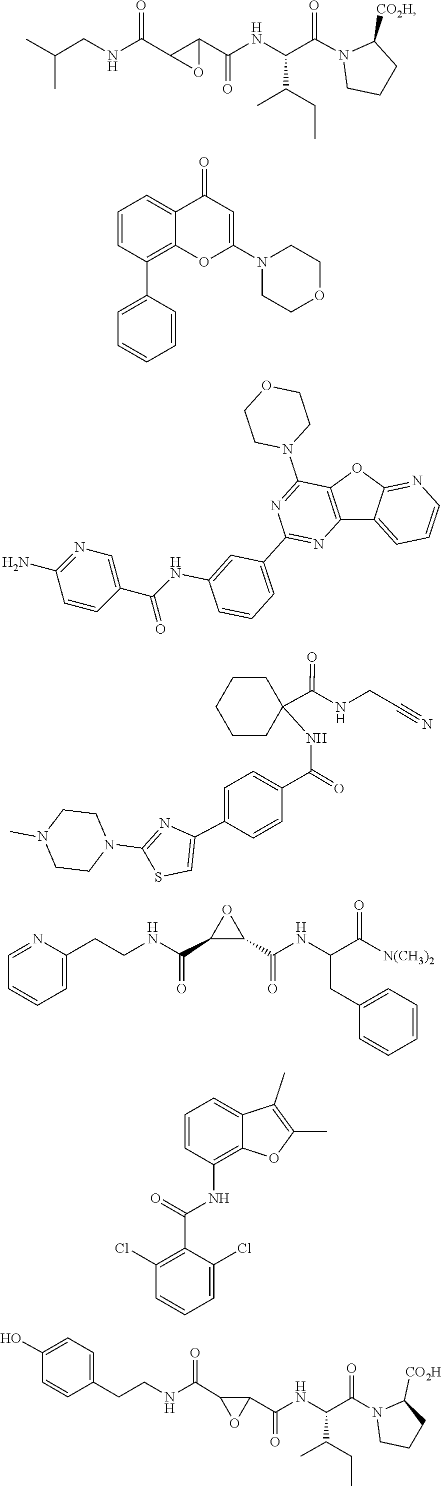

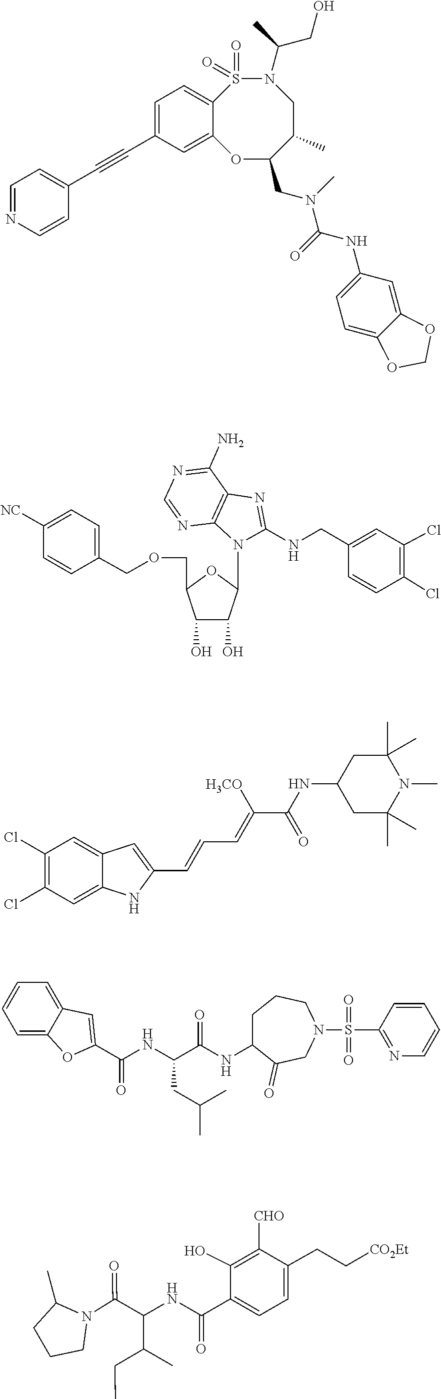 [SMILES: C[C@H]1CN([C@@H](C)CO)S(=O)(=O)c2ccc(C#Cc3ccncc3)cc2O[C@@H]1CN(C)C(=O)Nc1ccc2c(c1)OCO2 ; N#Cc1ccc(COCC2OC(n3c(NCc4ccc(Cl)c(Cl)c4)nc4c(N)ncnc43)[C@H](O)[C@@H]2O)cc1 ; CO/C(=C\C=C\c1cc2cc(Cl)c(Cl)cc2[nH]1)C(=O)NC1CC(C)(C)N(C)C(C)(C)C1 ; CC(C)C[C@H](NC(=O)c1cc2ccccc2o1)C(=O)NC1CCCN(S(=O)(=O)c2ccccn2)CC1=O ; CCOC(=O)CCc1ccc(C(=O)NC(C(=O)N2CCCC2C)C(C)CC)c(O)c1C=O]

In some embodiments, the ammonia suppressing agent includes a ASCT2 inhibitor. In some embodiments, the ammonia suppressing agent includes a ASCT2 inhibitor comprising V-9302, GPNA, benzylserine (BenSer), 2-amino-4-bis(aryloxybenzyl)aminobutanoic acid (AABA), or a

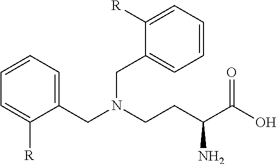

combination thereof. In some embodiments, the 2-amino-4-bis(aryloxybenzyl)aminobutanoic acids, for instance compounds having the formula:
wherein R is in each case independent selected from C₀₋₄alkaryl and C₀₋₄alkheteroaryl. When C is 0, the substituent is simply the aryl or heteroaryl, when C is 1, the substituent may be designated a benzyl group, etc. Exemplary aryl groups include unsubstituted and monosubstituted aryl wherein the substitution is selected from C₁₋₄alkyl, C₁₋₄haloalkyl, C₁₋₄alkoxy, F, Cl, COOH, CN. Specific substituents (when C is 0) include 2-methylphenyl, 3-methylphenyl, 4-methylphenyl, 2-methoxyphenyl, 3-methoxyphenyl, 4-methoxyphenyl, 2-trifluorophenyl, 3-trifluorophenyl, 4-trifluorophenyl, 2-chlorophenyl, 3-chlorophenyl, 4-chlorophenyl, 2-fluorophenyl, 3-fluorophenyl, and 4-fluorophenyl. In other embodiments, C is 1; specific substituents include 2-methylbenzyl, 3-methylbenzyl, 4-methylbenzyl, 2-methoxybenzyl, 3-methoxybenzyl, 4-methoxybenzyl, 2-chlorobenzyl, 3-chlorobenzyl, 4-chlorobenzyl, 2-fluorobenzyl, 3-fluorobenzyl, and 4-fluorobenzyl. Exemplary heteroaryl groups include pyridine-2-yl, pyridine-3-yl, pyridine-4-yl, preferably when C is 0 or 1.

In some embodiments, the ACST2 inhibitor is one of.

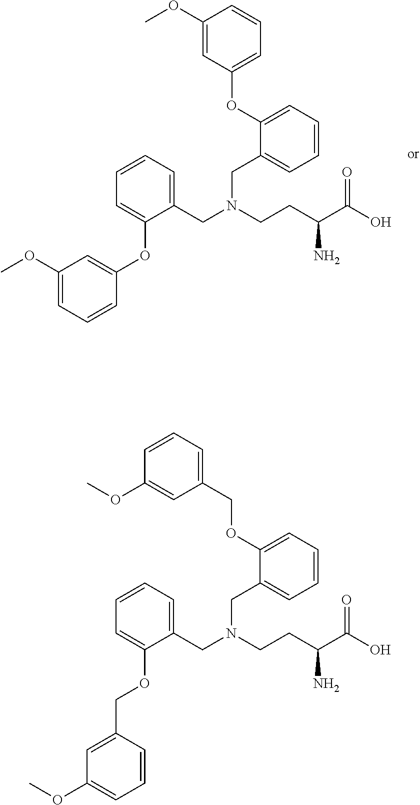 [SMILES: COc1cccc(Oc2ccccc2CN(CC[C@H](N)C(=O)O)Cc2ccccc2Oc2cccc(OC)c2)c1 ; COc1cccc(COc2ccccc2CN(CC[C@H](N)C(=O)O)Cc2ccccc2OCc2cccc(OC)c2)c1]

In some embodiments, the ACST2 inhibitor is

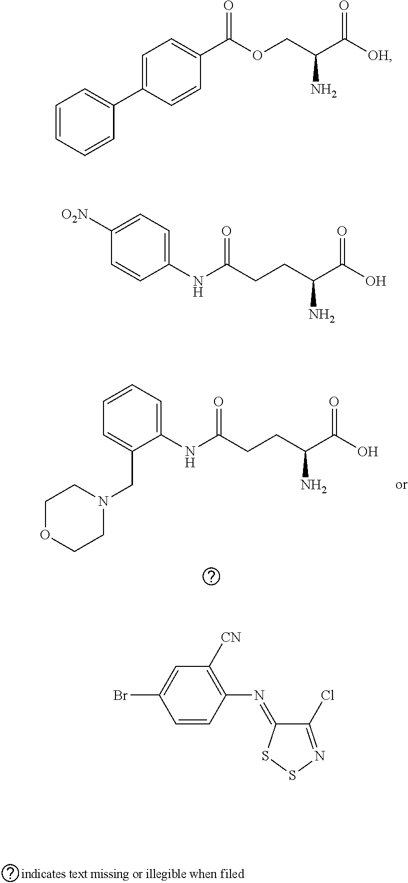 [SMILES: N[C@@H](COC(=O)c1ccc(-c2ccccc2)cc1)C(=O)O ; N[C@@H](CCC(=O)Nc1ccc([N+](=O)[O-])cc1)C(=O)O ; N[C@@H](CCC(=O)Nc1ccccc1CN1CCOCC1)C(=O)O ; N#Cc1cc(Br)ccc1/N=c1\ssnc1Cl]

In some embodiments the ammonia suppressing agent includes a glutaminase inhibitor. In some embodiments, the ammonia suppressing agent is includes GLS2 inhibitor. In some embodiments, the ammonia suppressing agent is includes GLS1 inhibitor.

In some embodiments, the ammonia suppressing agent includes a glutaminase inhibitor includes 6-diazo-5-oxonorleucine (aka “DON”), bis-2-(5-phenylacetamido-1,3, 4-thiadiazol-2-yl) ethyl sulphide (“BPES”), 5-(3-bromo-4-(dimethylamino)phenyl)-2,2-dimethyl-2,3,5,6-tetrahydrobenzo[a]phenanthridin-4(1H)-one, telaglenastat (“CB-839”), ethyl 2-(2-amino-4-methylpentanamido)-6-diazo-5-oxonorleucine, IPN60090, e.g., GK921; UPGL00004; BPTES1 JHU-083, or combinations thereof, as well as compounds having the formula.

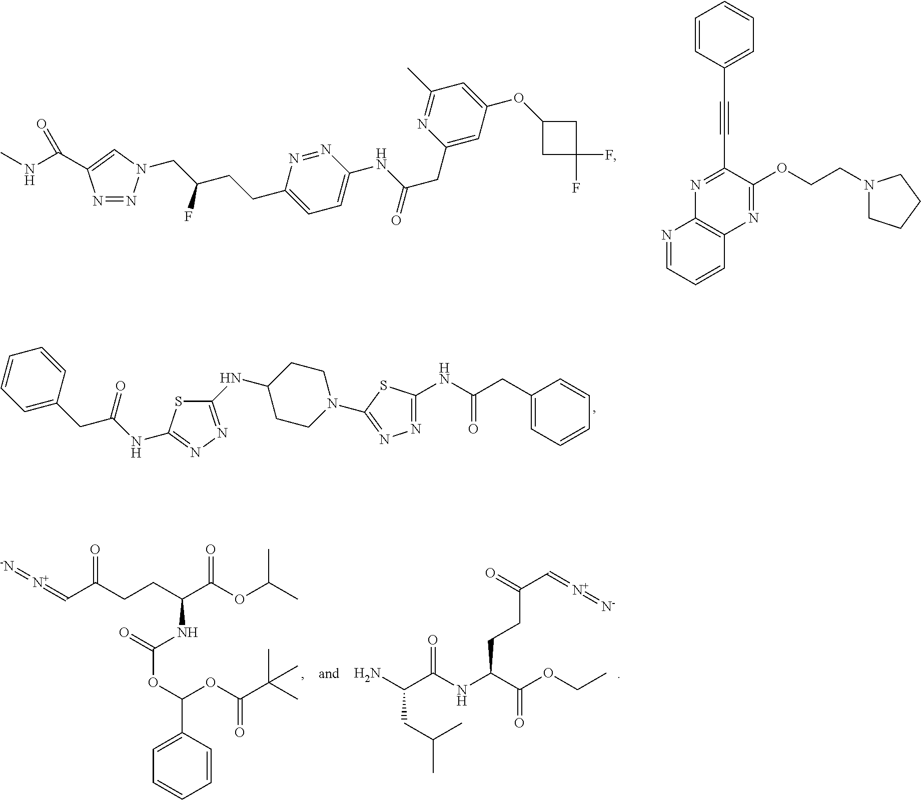

In some embodiments, the SREBP inhibitor includes a SRBEP-2 inhibitor. In some embodiments, the SREBP inhibitor is a S2P inhibitor, a S1P inhibitor, a SQLE inhibitor, a fatty acid synthesis pathway inhibitor, a SCD1 inhibitor, an HMG-CoA inhibitor, a FASN inhibitor, or combinations thereof. In some embodiments, the SREBP inhibitor includes fatostatin, tocotrienol, artesunate, ursolic acid, archazolid B, PF-429242, nelfinavir, cinobufotalin, 24yridin; 1-(4-bromophenyl)-3-(25yridine-3-yl)urea, firsocostate, YTX-7739, TVB-2640, PF-05221304; ND646; PF-05175157, CP 640186, NB-598, terbinafine, or a combination thereof.

Exemplary HMG-CoA inhibitors include cerivastatin, itavastatin, pitavastatin, simvastatin, simvastatin acid, mevastatin, 3′-hydroxy simvastatin acid, 6′-hydroxymethyl simvastatin acid, lovastatin, atorvastatin. Fluvastatin, pravastatin, and rosuvastatin.

In some embodiments, the SREBP inhibitor includes a compound having the formula:

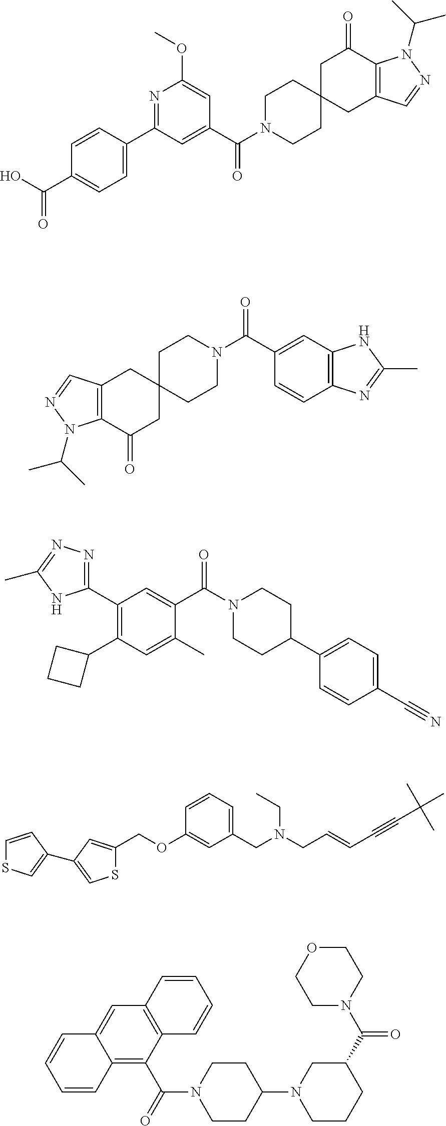

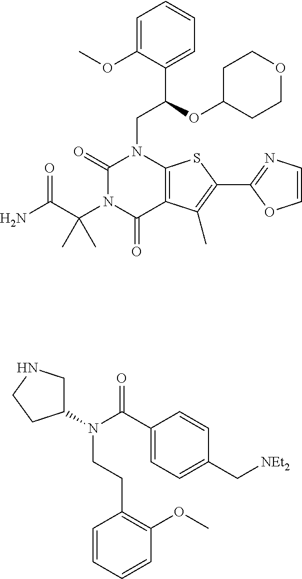

In some embodiments, the at least one ammonia suppressing agent, SREBP inhibitor, fatty acid synthesis inhibitor, or cholesterol synthesis pathway inhibitor and lysosomal dysregulating agent are administered concurrently. In some embodiments, a first ammonia suppressing agent, SREBP inhibitor, fatty acid synthesis inhibitor, or cholesterol synthesis pathway inhibitor is administered over the course of a first period of time, and a lysosomal dysregulating agent is administered over the course of a second period of time.

In some embodiments, the first ammonia suppressing agent, SREBP inhibitor, fatty acid synthesis inhibitor, or cholesterol synthesis pathway inhibitor is administered for a period of 1-28 days, 7-28 days, 14-28 days, 21-28 days, 1-21 days, 7-21 days, 14-21 days, 1-14 days, 7-14 days, 1-10 days, 2-10 days, 5-10 days, 1-7 days, 2-7 days, 1-5 days, 1-4 days, 1-3 days, 1-2 day, or 1 day.

In some embodiments, the lysosomal dysregulating agent is administered for a period of 1-28 days, 7-28 days, 14-28 days, 21-28 days, 1-21 days, 7-21 days, 14-21 days, 1-14 days, 7-14 days, 1-10 days, 2-10 days, 5-10 days, 1-7 days, 2-7 days, 1-5 days, 1-4 days, 1-3 days, 1-2 day, or 1 day.

In some embodiments, the method includes further administering at least one additional anti-cancer agent to the subject. In some embodiments, the method includes further administering to the subject at least one additional anti-cancer agent including Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aligopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil--Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil--Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil--Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil--Topical), Fluorouracil Injection, Fluorouracil--Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin). Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hvaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritale (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil--Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

In some embodiments, the cancer includes acute lymphoblastic leukemia (ALL), acute myeloid leukemia (AML), cancer in adrenocortical carcinoma, adrenal cortex cancer, AIDS-related cancers, Kaposi sarcoma, AIDS-related lymphoma, primary CNS lymphoma, anal cancer, appendix cancer, carcinoid tumors, astrocytomas, atypical teratoid/rhabdoid tumor, basal cell carcinoma, skin cancer (nonmelanoma), bile duct cancer, extrahepatic bladder cancer, bladder cancer, bone cancer (includes Ewing sarcoma and osteosarcoma and malignant fibrous histiocytoma), brain tumors, breast cancer, bronchial tumors, Burkitt lymphoma (non-Hodgkin), carcinoid tumor, cardiac (heart) tumors, atypical teratoid/rhabdoid tumor, embryonal tumors, germ cell tumors, lymphoma, primary-cervical cancer, cholangiocarcinoma, chordoma, chronic lymphocytic leukemia (CLL), chronic myelogenous leukemia (CML), chronic myeloproliferative neoplasms, colorectal cancer, colorectal cancer, craniopharyngioma, cutaneous T-cell lymphoma, ductal carcinoma in situ (DCIS), embryonal tumors, central nervous system, endometrial cancer, ependymoma, esophageal, esthesioneuroblastoma, ewing sarcoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastric (stomach) cancer, gastrointestinal carcinoid tumor, gastrointestinal stromal tumors (GIST), gastrointestinal stromal tumors (GIST), germ cell tumors, central nervous system, extracranial, extragonadal, ovarian testicular, gestational trophoblastic disease, gliomas, hairy cell leukemia, head and neck cancer, heart tumors, hepatocellular (liver) cancer, histiocytosis, Langerhans Cell, Hodgkin's lymphoma, hypopharyngeal cancer, intraocular melanoma, islet cell tumors, pancreatic neuroendocrine tumors, Kaposi sarcoma, kidney-langerhans cell histiocytosis, laryngeal cancer, laryngeal cancer and papillomatosis, leukemia, lip and oral cavity cancer, liver cancer (primary), lung cancer, lung cancer, lymphoma-macroglobulinemia, Waldenström -Non-Hodgkin lymphoma, male breast cancer, malignant fibrous histiocytoma of bone and osteosarcoma, melanoma, intraocular (eye), Merkel cell carcinoma, mesothelioma, malignant, mesothelioma, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (CML), myeloid leukemia, acute (AML), nasal cavity and paranasal sinus cancer, nasopharyngeal cancer, nasopharyngeal cancer, neuroblastoma, non-Hodgkin lymphoma, non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), papillomatosis, paraganglioma, paraganglioma, paranasal sinus and nasal cavity cancer, parathyroid cancer, penile cancer, pharyngeal cancer, pheochromocytoma, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, retinoblastoma, rhabdomyosarcoma, salivary gland cancer, salivary gland tumors, Ewing sarcoma, Kaposi sarcoma, osteosarcoma, rhabdomyosarcoma, uterine sarcoma, vascular tumors, Sézary syndrome, skin cancer, small cell lung cancer, small intestine cancer, soft tissue sarcoma, squamous cell carcinoma, squamous neck cancer with occult primary, metastatic, stomach (gastric) cancer, stomach (gastric) cancer, T-cell lymphoma, cutaneous, testicular cancer, throat cancer, thymoma and thymic carcinoma, thyroid cancer, ureter and renal pelvis, transitional cell cancer, urethral cancer, uterine cancer, endometrial and uterine sarcoma, vaginal cancer, vaginal cancer, vascular tumors, vulvar cancer, Waldenstrom Macroglobulinemia, or Wilms Tumor.

It should be understood that the method includes administering a composition, compound, or formula in such amounts, time, and route deemed necessary in order to achieve the desired result. The exact amount will vary from subject to subject, depending on the species, age, and general condition of the subject, the severity of the cancer, the particular composition, compound, or formula, its mode of administration, its mode of activity, and the like. The composition, compound, or formula are preferably formulated in dosage unit form for ease of administration and uniformity of dosage. It will be understood, however, that the total daily usage of the composition, compound, or formula will be decided by the attending physician within the scope of sound medical judgment. The specific therapeutically effective dose level for any particular subject will depend upon a variety of factors including the cancer being treated and the severity of the cancer; the activity of the composition, compound, or formula employed; the specific composition, compound, or formula employed; the age, body weight, general health, sex and diet of the patient; the time of administration, route of administration, and rate of excretion of the specific composition, compound, or formula employed; the duration of the treatment; drugs used in combination or coincidental with the specific composition, compound, or formula employed; and like factors well known in the medical arts.

The composition, compound, or formula may be administered by any route. In some embodiments, the composition, compound, or formula is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition, compound, or formula (e.g., its stability in the environment of the gastrointestinal tract), the condition of the subject (e.g., whether the subject is able to tolerate oral administration), etc.

The exact amount of a composition, compound, or formula required to achieve a therapeutically effective amount will vary from subject to subject, depending on species, age, and general condition of a subject, severity of the side effects or disorder, identity of the particular composition(s), compound(s), or formula(s), mode of administration, and the like. The amount to be administered to, for example, a child or an adolescent can be determined by a medical practitioner or person skilled in the art and can be lower or the same as that administered to an adult.

In one aspect, disclosed herein is a kit comprising a first agent comprising an ammonia suppressing agent and another agent comprising one or more lipid metabolism inhibitors, or related agents thereof.

In some embodiments, the lipid metabolism inhibitor according to any preceding aspect includes at least one inhibitor suppressing the utilization/release of lipids from extracellular uptake (LDL) and internal storage (lipid droplets, LDs).

In some embodiments, the inhibitors suppressing the utilization/release of lipids from extracellular uptake and internal storage according to any preceding aspect includes a SREBP inhibitor, fatty acid synthesis inhibitor, cholesterol synthesis pathway inhibitor, or lysosome dysregulating agent.

In some embodiments, the kit includes at least one ammonia suppressing agent and at least one SREBP inhibitor according to any preceding aspect.

In some embodiments, the kit includes at least one ammonia suppressing agent and at least one fatty acid synthesis inhibitor according to any preceding aspect.

In some embodiments, the kit includes at least one ammonia suppressing agent and at least one cholesterol synthesis pathway inhibitor according to any preceding aspect.

In some embodiments, the kit includes at least one ammonia suppressing agent and at least one lysosome dysregulating agent according to any preceding aspect.

In some embodiments, the kit includes at least one ammonia suppressing agent, at least one lysosome dysregulating agent, and at least one SREBP inhibitor according to any preceding aspect.

In some embodiments, the kit includes at least one ammonia suppressing agent, at least one lysosome dysregulating agent, and at least one fatty acid synthesis inhibitor according to any preceding aspect.

In some embodiments, the kit includes at least one ammonia suppressing agent, at least one lysosome dysregulating agent, and at least one cholesterol synthesis pathway inhibitor according to any preceding aspect.

In one aspect, disclosed herein is a pharmaceutical composition including at least one ammonia suppressing agent alone or in combination with one or more lipid metabolism inhibitors, or related agents thereof.

In some embodiments, the pharmaceutical composition includes the one or more lipid metabolism inhibitors includes at least one inhibitor suppressing the utilization/release of lipids from extracellular uptake (LDL) and internal storage (lipid droplets, LDs) according to any preceding aspect.

In some embodiments, the inhibitors suppressing the utilization/release of lipids from extracellular uptake and internal storage includes a SREBP inhibitor, fatty acid synthesis inhibitor, cholesterol synthesis pathway inhibitor, or lysosome dysregulating agent according to any preceding aspect.

In some embodiments, the pharmaceutical composition includes at least one ammonia suppressing agent and at least one SREBP inhibitor according to any preceding aspect.

In some embodiments, the pharmaceutical composition includes at least one ammonia suppressing agent and at least one fatty acid synthesis inhibitor according to any preceding aspect.

In some embodiments, the pharmaceutical composition includes at least one ammonia suppressing agent and at least one cholesterol synthesis pathway inhibitor according to any preceding aspect.

In some embodiments, the pharmaceutical composition includes at least one ammonia suppressing agent and at least one lysosome dysregulating agent according to any preceding aspect.

In some embodiments, the pharmaceutical composition includes at least one ammonia suppressing agent, at least one lysosome dysregulating agent, and at least one SREBP inhibitor according to any preceding aspect.

In some embodiments, the pharmaceutical composition includes at least one ammonia suppressing agent, at least one lysosome dysregulating agent, and at least one fatty acid synthesis inhibitor according to any preceding aspect.

In some embodiments, the pharmaceutical composition includes at least one ammonia suppressing agent, at least one lysosome dysregulating agent, and at least one cholesterol synthesis pathway inhibitor according to any preceding aspect.

In one aspect, disclosed herein is a method of treating a solid tumor in a patient in need thereof, including administering to the patient at least one ammonia suppressing agent, SREBP inhibitor, fatty acid synthesis inhibitor, or cholesterol synthesis pathway inhibitor alone or in combination with inhibitors suppressing the utilization/release of lipids from extracellular uptake and internal storage.

In one aspect, disclosed herein is a method of inhibiting lipogenesis in a patient in need thereof, including administering to the patient at least one ammonia suppressing agent, SREBP inhibitor, fatty acid synthesis inhibitor, or cholesterol synthesis pathway inhibitor alone or in combination with inhibitors suppressing the utilization/release of lipids from extracellular uptake and internal storage.

In one aspect, disclosed herein is a method of increasing reactive oxygen species in a patient in need thereof, including administering to the patient at least one ammonia suppressing agent, SREBP inhibitor, fatty acid synthesis inhibitor, or cholesterol synthesis pathway inhibitor alone or in combination with inhibitors suppressing the utilization/release of lipids from extracellular uptake and internal storage.

In one aspect, disclosed herein is a method of causing mitochondrial damage in a patient in need thereof, including administering to the patient at least one ammonia suppressing agent, SREBP inhibitor, fatty acid synthesis inhibitor, or cholesterol synthesis pathway inhibitor alone or in combination with inhibitors suppressing the utilization/release of lipids from extracellular uptake and internal storage.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Ammonia is A Key Activator Stimulating Scap/Insig Dissociation and Srebp-1 Activation to Promote Lipogenesis and Tumor Growth

### Experimental Procedures

Reagents. Antibodies for SCAP (9D5) (#sc-69836), PDI (H-17) (#sc-30932) and Lamin A (H-102) (#sc-20680) were purchased from Santa Cruz Biotechnology. SCAP antibody (#A303-554A) was from Bethyl Laboratories, Inc. SREBP-2 (#557037) and SREBP-1 (IgG-2A4) (557036) antibodies for western blot were purchased from BD Pharmingen. SREBP-1 (2A4) (#ab3259), GLS (#ab93434) and Giantin (#ab24586) antibodies for immunofluorescence (IF) were from Abcam. Antibodies for ASPG (#HPA069761) and SDS (#LS-C173534) were from Sigma and Lifespan Biosciences, respectively. Antibodies for GFP (#11814460001), FLAG-tag (#F3165), p-EGFR Y1086 (#369700) and EGFR (#05-1047) were purchased from Roche, Sigma, Invitrogen, and Millipore, respectively. Antibodies for FASN (#3180S), SCD1 (M38) (#2438S), HA-tag (C29F4) (#3724S), p-Akt Thr308 (#9275S), Ser473 (587F11) (#4051S), Akt (pan) (C67E7) (#4691S), BiP (C50B12) (3177s) and Grp94 (20292S) were purchased from Cell Signaling. Antibodies for Ribophorin I (PIPA527562) was purchased from Fisher. Antibodies for ERGIC-53 (rat homolog, p58) (E1031) was purchased from Sigma. Glucose (#G8644), sodium L-lactate (#L7022), α-Ketoglutaric acid sodium salt (#K1875), L-Glutamic acid monosodium salt monohydrate (#49621), and ammonium hydroxide solution (#318612) were from Sigma. L-glutamine (#25030-081) and sodium pyruvate (#11360-070) were from Life Technologies. Ammonium chloride (#12125-02-9), GPNA (gamma-L-Glutamyl-p-nitroanilide Hydrochloride) (#151495), and RPMI1640 with 2 g/L sodium bicarbonate and without L-glutamine and glucose (#091646854) were from MP Biomedicals. CB-839 (#A14396-5) was from AdooQ Bioscience, Pepstatin A (#P5318), Leupeptin (#L2884), and human EGF (#E9644) were purchased from Sigma. Dulbecco's modified Eagle's medium (DMEM) without glucose, pyruvate, glutamine (#17-207-CV) and DPBS (21-030-CV) were purchased from Corning. Cholesterol-Water Soluble (#C4951), 25-Hydroxycholesterol (25-HC) (#H1015) and GTP (10106399001) were purchased from Sigma. Hanks' Balanced Salt Solution (HBSS) (#14170) was purchased from Life Technologies. Octyl-α-KG (SML2205), L-Histidine monohydrochloride

(H5659), L-Isoleucine (I7403), L-Leucine (L8912), L-Lysine monohydrochloride (L8662), L-Methionine (M5308), L-Phenylalanine (P5482), L-Threonine (T8441), L-Tryptophan (T8941), L-Valine (V0513), L-Aspartic acid (A7219), L-Asparagine monohydrate (A8381), L-Arginine monohydrochloride (A6969), L-Tyrosine (T8566), and L-Cystine dihydrochloride (C6727) were purchased from Sigma. Ammonia Assay Kit (ab83360), Glutamate Assay Kit (ab138883) and α-ketoglutarate (α-KG) Assay Kit (ab83431) were purchased from Abcam. The ATG5 siRNA (sc-41445) was purchased from Santa Cruz. The siRNAs for GDH1 (cat #L-004032-00-0005), GDH2 (cat #L-009067-01-0005), ASPG (cat #E-030336-00-0005) and SDS (cat #L-008214-01-0005) were purchased from Dharmacon. Creatine kinase (CK) (10127566001), Sodium creatine phosphate dibasic tetrahydrate (27920), Sorbitol (56755), Adenosine 5′-triphosphate disodium salt hydrate(A7699), and Hexyl β-D-glucopyranoside (53180) were from Sigma.

Clinical Samples. Individual lung tumor and adjacent normal tissues, lung tumor tissue microarray (TMA) containing 50 paired (tumors and matched adjacent normal lung tissues) and 49 unpaired lung tumor tissues, and individual GBM tumor tissues were from the Department of Pathology at The Ohio State University. All human tissues were collected from Ohio State University Hospitals under Institutional Review Board-(IRB) and HIPPA-approved protocols, and histologically confirmed. Glioma TMA with 91 tumors was from the University of Kentucky and IRB approval was obtained at UK prior to study initiation. All samples had tested negative for HIV and hepatitis B. TMA slides were scanned using ScanScope and analyzed using ImageScope v11 software (Aperio Technologies, Vista, CA, USA). The staining intensity of tissues was graded as 0, 1+, 2+, or 3+. H-score was calculated using the following formula: H score=[1×(% cells with 1+)+2×(% cells with 2+)+3×(% cells with 3+)]×100.

Plasmids, pCMV-Myc-Insig-1, pcDNA3.1-2×Flag-SREBP-1a (full length) and -1c (full length), pcDNA3.0-HA-SREBP-2 (full length), and pcDNA3.0-GFP-SCAP (QQQ) plasmids were obtained or cloned as previously described28. pcDNA3.0-GFP-SCAP wild-type plasmid was a gift from Dr. Peter Espenshades from Johns Hopkins University. The pcDNA3.0-GFP-SCAP (D428A) was constructed by PCR from the pcDNA3.1-SCAP D428A plasmid provided by Drs. Brown and Goldstein from the University of Texas Southwestern Medical Center6l. The other four single-point-mutants, including pcDNA3.0-GFP-SCAP-(D428E), -(D428N), -(D428K), -(S326A), -(S330A), -(S326A/S330A) and -(V353G) were constructed using site-directed mutagenesis (Q5 Site-Directed Mutagenesis Kit, #E0554S, NEB).

Cell Culture and Transfection. U87, U87EGFR, LN229, T98, M233, HepG2, HEK293T, and MDA468 were maintained in DMEM (#15-013-CV, Cellgro). H1299, H1975, HCC4006, and H1299-luc cell lines were cultured in RPMI-1640 medium (#15040CV, Cellgro). All media were supplemented with 5% HyClone fetal bovine serum (FBS, #SH30071.03, GE Healthcare) and 4 mM Glutamine (#25030-081, Life Technologies). GBM30, primary GBM patient-derived cells were maintained in DMEM/F12 (#MT90090PB, Fisher) containing B-27 serum-free supplements (1×), heparin (2 mg/ml), EGF (50 ng/ml), glutamine (2 mM) and fibroblast growth factor (FGF, 50 ng/ml). All cell lines were cultured in a humidified atmosphere of 5% C02 at 37° C. Transfection of plasmids was performed using X-tremeGENE HP DNA Transfection Reagent (#06366236001, Roche) following the manufacturer's instructions.

Cell Proliferation. A total of 2×104 cells was seeded in 12-well plates, and washed with PBS after 24 hr, followed by addition of fresh medium with 1% dialyzed FBS (#35-071-CV, Cellgro), and supplemented with or without 5 mM glucose or/and 4 mM glutamine for 4 days. Live cells were counted at the indicated times using a hemocytometer after trypan blue staining.

Western Blot. Cells were lysed with RIPA buffer containing a protease inhibitor cocktail and phosphatase inhibitors. The proteins were separated on 12% SDS-PAGE, and transferred onto an ECL nitrocellulose membrane (#1620112, Bio-Rad). After blocking for 1.5 hr in 5% non-fat dried milk diluted by Tris-buffered saline containing 0.1% Tween 20, the membranes were incubated with various primary antibodies, followed by appropriate secondary antibodies conjugated to horseradish peroxidase. Immunoreactivity was revealed using an ECL kit (#RPN2106, GE Healthcare).

Quantitative Real-time PCR. Total RNA was isolated with TRIzol according to the manufacturer's protocol, and cDNA was synthesized with the iScript cDNA Synthesis Kit. Quantitative real-time PCR was performed with iQ SYBR Green Supermix using the Applied Biosystems (ABI) 7900HT Real-Time PCR System. The expression was normalized to the 36B4 housekeeping gene and calculated with the comparative method (2-AACt).

MISSION pLKO.1-puro lentivirus vectorscontaining shRNA for SREBP-1 (#1: TRCN0000414192; #2: TRCN0000421299), shSREBP-2 (TRCN0000020665), shGLS (#1: TRCN0000051135; #2: TRCN0000051137) and non-mammalian shRNA control (#SHC002) were purchased from Sigma. The 293FT cells were transfected with shRNA vector and packing plasmids psPAX2 (#12260, Addgene) and the envelope plasmid pMD2.G (#12259, Addgene) using polvethyleneimine (#23966; Polysciences). Supernatants were harvested after 48 hr and 72 hr and concentrated using the Lenti-X Concentrator (#631232; Clontech) according to the protocol. The virus titer was quantified by real-time PCR using the qPCR Lentivirus Titration Kit. Lentiviral transduction was performed according to the Sigma MISSION protocol with polybrene (8 g/ml).

Cells were infected with the same multiplicity of infection (MOI) of shRNA. siRNA Knockdown. After the cells were seeded and cultured in full medium supplemented with 5% FBS for 24 hr, the related siRNA targeting ATG5, GDH1/2, ASPG, or SDS were transfected into H1299 cells using lipofectamine RNAiMAX (13778-150, Invitrogen) for 24 hr. The cells were then washed with PBS once and treated as described in each experiment for 12 hr. The treated cells were harvested and extracted for real-time qRT-PCR and Western Blot analysis.

RNA Sequencing. Total RNA from treated H1299 cells was extracted using the Total RNA Purification Plus kit (#48300, NORGEN BIOTEK CORP., Canada), followed by quality assessment by NanoDrop One (#70-105-8111, Thermo Fisher Scientific, USA). For mRNA library generation, 200 ng of total RNA was treated with NEBNext Poly mRNA Magnetic Isolation Module (#E7490L, New England Biolabs, USA) following the manufacturer's protocol. Subsequently, isolated mRNA was fragmented for 10 min. cDNA was synthesized and amplified for 12 PCR cycles using NEBNext Ultra II Directional (stranded) RNA Library Prep Kit for Illumina (#E7760L, New England Biolabs, USA) with NEBNext Multilex Oligos Indexes kit following the manufacturer's directions. Distributions of the template length and adapter-dimer contamination were assessed using an Agilent 2100 Bioanalyzer (#G2939BA, Agilent Technologies, Inc) and High Sensitivity DNA kit (#5067-4626, Agilent Technologies, Inc). The average template length was approximately 150 bp. Contamination of adapter-dimers was negligible. The concentration of cDNA libraries was determined using Invitrogen Qubit dsDNA HS reagents (#32851, Invitrogen) and read on a Qubit Fluorometer (#Q33238, Thermo Fisher), and cDNA libraries were paired end sequenced on a NovaSeq6000 SP 300 cycles (<2×150 bp) (Illumina, USA). Raw data were mapped via HISAT2 v2.1.0 to the human reference genome (GRCh38p12). Differentially expressed genes (DEGs) were called using the limma-voom method. Gene expression fold change, false discovery rate (FDR), and p values were calculated. Highly significant DEGs (p value <10-6) were subjected to pathway analysis through Ingenuity Pathway Analysis (IPA) (QIAGEN Bioinformatics). Enriched metabolomics pathways were ranked by Z-scores. The top 20 pathways were visualized by heatmaps, generated by MeV4.9. RNA-seq was performed by the OSUCCC Genomics Shared Resource.

Co-immunoprecipitation. Co-immunoprecipitations were performed as previously described28. Briefly, HEK293T cells were transiently transfected with pcDNA3.0-GFP, pcDNA3.0-GFP-SCAP wild-type or pcDNA3.0-GFP-SCAP (D428A) together with/without pCMV-Myc-Insig-1 using X-tremeGENE HP DNA Transfection Reagent. At 24 hr post-transfection, cells were washed once with ice-cold PBS and lysed with 0.5 ml of immunoprecipitation (IP) lysis buffer (50 mM HEPES-KOH, pH 7.4, 100 mM NaCl, 1.5 mM MgCl2, 0.1% Nonidet P-40, 1 g/ml pepstatin A, 10 μg/ml leupeptin, and 2 g/ml aprotinin). Cell lysates were passed through a 22-gauge needle 15 times and incubated for 1 hr at 4° C. The cell extracts were clarified by centrifugation at 20,000×g for 30 min at 4° C. Supernatants were pre-cleared for 1 hr by rotation with 30 l of pre-equilibrated protein G-agarose beads at 4° C. (#11243233001, Roche Applied Science). Pre-cleared lysates were incubated with 2 g of anti-GFP antibody at 4° C. for 1 hr, 30 l of pre-equilibrated protein G-agarose beads were then added and rotated for 16 hr at 4° C. After centrifugation, the beads were washed three times with 1 ml of ice-cold IP lysis buffer. The bead-bound proteins were eluted by boiling in SDS-PAGE sample buffer and subjected to SDS-PAGE and subsequent western blot analysis.

Analysis of Metabolites in Cell Culture Medium. Metabolite levels in culture medium, including glucose, glutamine, lactate, glutamate, and NH4+, were measured using the Nova Bioprofile 100 Plus Bioanalyzer (Nova Biomedical). H1299 (4×105) or U87 (3×105) cells were seeded in 60 mm dish for 24 hr. After the cells were washed with PBS, they were switched to 2.5 ml serum-free medium with glucose (5 mM) and glutamine (4 mM) for 12 hr. The culture or control media (without cultured cells) were centrifuged at 12,000 rpm for 1 min and run on the bioanalyzer. Cell numbers were determined by using a hemocytometer after trypan blue staining. Consumption of glucose and glutamine or production of lactate, glutamate, and NH4+ under each experimental condition was calculated by subtracting their levels in control medium and normalizing to cell numbers.

Measures of Ammonia Levels in Tissues, Cells and bound to SCAP. To measure ammonia levels in tumors and normal tissues, 10 mg of tissues were collected and lysed through homogenization on ice in the ammonia assay buffer (100 l) from the commercial kit. The lysates were centrifuged at 20,000×g for 5 min at 4° C. and the ammonia levels in the supernatants were measured by the ammonia assay kit following the manufacturer's instructions (#ab83360, Abcam). For measurement of SCAP-bound ammonia, a total of 1.3×107 HEK293T cells was seeded in 15 cm dishes for 24 hr. The cells were transfected with GFP, GFP-SCAP wild-type, or D428A mutant together with myc-Insig1 plasmids for 24 hr, and then washed with PBS, followed by addition of fresh DMEM medium containing glucose (5 mM) and NH4Cl (4 mM) for 2 hr in the absence of glutamine. The cells were then washed with ice-cold PBS and lysed with 1 ml of buffer (25 mM Tris, pH 8.0, 150 mM NaCl, 1% (w/v) LMNG (DL14035, Biosynth Carbosynth) containing a protease inhibitor cocktail50. Cell lysates were passed through a 22-gauge needle 30 times and incubated for 1 hr at 4° C. The cell extracts were clarified by centrifugation at 17,000×g for 10 min at 4° C. Supernatants were incubated for 1 hr by rotation with 50 μl of pre-equilibrated GFP-Trap agarose beads (#gta, ChromoTek) at 4° C. The precipitated protein complex was washed with 1 ml buffer (25 mM Tris, pH 8.0, 150 mM NaCl, 0.005% (w/v) LMNG) twice, and then added to 50 μl ammonia assay buffer to measure ammonia according to the kit instructions. Measurements of ammonia, glutamate and α-KG levels in cells were conducted using the ammonia assay kit, glutamate assay kit (ab138883) and α-KG assay kit (ab83431), respectively, according to the manufacturer's instructions.

Immunohistochemistry. Tissue sections were cut from biopsy paraffin blocks. Tissue slides were placed in an oven at 60° C. for 30 min., and then deparaffinized by incubating with xylene three times for 5 min. each, followed by dipping in graded alcohols (100%, 95%, 80%, and 70%) three times for 2 min. each. Slides were washed with distilled water (dH2O) 3 times for 5 min., and then immersed in 3% hydrogen peroxide for 10 min, followed by washes with dH2O. Slides were transferred into preheated 0.01 M citrate buffer (pH 6.0) in a steamer for 30 min., and then washed with dH2O and PBS after cooling. Slides were blocked with 3% BSA/PBS at room temperature for 1 hr and then incubated with primary antibody overnight at 4° C., followed by incubation at room temperature for 30 min with the appropriate secondary antibody, including Biotinylated Anti-rabbit IgG and Biotinylated Anti-mouse IgG. After incubation with avidin-biotin complex followed by washing (3×5 min.) with PBS and staining with NovaRed solution, slides were washed with tap water, counterstained with hematoxylin and dipped briefly in graded alcohols (70%, 80%, 95% and 100%) and in xylene 2×5 min. Finally, slides were mounted and imaged with SPOT 5.2 (SPOT IMAGING) or scanned with the Aperio Scanscope XT scanner (Leica).

Immunofluorescence. Cells grown on coverslips were washed with PBS twice and fixed with 4% formaldehyde for 15 min, then permeabilized with 0.1% Triton X-100/PBS for 5 min and blocked by 3% bovine serum albumin for 30 min at room temperature. The cells were stained with primary antibodies overnight at 4° C. or for 30 min at 37° C., followed by incubation with Alexa Fluor 568-labeled goat anti-rabbit IgG (H+L) (#A11036, Invitrogen) for 30 min at 37° C. Cells were washed three times with PBS in a dark chamber. The coverslips were washed as described above, inverted, mounted on slides using ProLong Gold antifade reagent with DAPI (#2188179, Invitrogen) and examined with a Zeiss LSM510 Meta confocal microscopy.

Preparation of Cell Membrane Fractions and Nuclear Extracts. Cells were washed once with PBS, scraped into 1 ml PBS, and centrifuged at 1000×g for 5 min at 4° C. Cells were then suspended in ice-cold buffer containing 10 mM HEPES-KOH (pH 7.6), 10 mM KCl, 1.5 mM MgCl₂, and 1 mM sodium EDTA, 1 mM sodium EGTA, 250 mM sucrose and a mixture of protease inhibitors (5 μg/ml pepstatin A, 10 μg/ml leupeptin, 0.5 mM PMSF, 1 mM DTT, and 25 μg/ml ALLN) for 30 min on ice. Extracts were then passed through a 22G×1½ needle 30 times and centrifuged at 890×g at 4° C. for 5 min to isolate the nuclei. The nuclear pellet was re-suspended in 0.1 ml of buffer C (20 mM HEPES/KOH pH 7.6, 0.42 M NaCl, 2.5% (v/v) glycerol, 1.5 mM MgCl2, 1 mM sodium EDTA, 1 mM sodium EGTA), and a mixture of protease inhibitors (5 μg/ml pepstatin A, 10 μg/ml leupeptin, 0.5 mM PMSF, 1 mM DTT, and 25 μg/ml ALLN). The suspension was rotated at 4° C. for 60 min and centrifuged at 20,000×g in a microcentrifuge for 20 min at 4° C. The supernatant was designated as “nuclear extracts.” The nuclear extracts were heated at 100° C. for 10 min with 5×loading buffer before being subjected to SDS-PAGE.

The supernatant from the 890×g spin was centrifuged at 20,000×g for 20 min at 4° C., and the pellet was dissolved in 0.1 ml of SDS lysis buffer (10 mM Tris-HCl pH 6.8, 100 mM NaCl, 1% (v/v) SDS, 1 mM sodium EDTA, and 1 mM sodium EGTA), incubated at 37° C. for 30 min, and designated as “membrane fraction”. The protein concentration was determined by pierce rapid gold BCA protein assay kit (A53225, Thermo Scientific). A bromophenol blue solution (1 μl 100×) was added to each sample before being subjected to SDS-PAGE and subsequent western blot analysis.

Preparation of Rat Liver Cytosol. Male Sprague-Dawley rats (350-400×g) were anesthetized by isoflurane (1349003, Sigma) inhalation following an intraperitoneal injection of buprenorphine (1078700, Sigma) and carprofen (PHR1452, Sigma), after which their livers were perfused with 0.9% (w/v) NaCl (R5201-01, B. Braun) through the portal vein. The livers were excised and disrupted in 2 ml/g of ice-cold Buffer A (50 mM HEPES-KOH (pH 7.2), 250 mM sorbitol, 70 mM KOAc, 5 mM potassium EGTA, 2.5 mM Mg(OAc)2, and protease inhibitors) supplemented with 1 mM dithiothreitol (43819, sigma), and then followed by 10 strokes in a Dounce homogenizer fitted with a Teflon pestle. Homogenates were centrifuged at 1000×g for 10 min. Supernatants were sequentially centrifuged at 20,000×g for 20 min, 186,000×g for 1 hr, and 186,000×g for 45 min. Following the 186,000×g spins, the fat layer was removed carefully before collecting the aqueous supernatant. All steps were carried out at 4° C. The final supernatant, considered as cytosol (total protein concentration: 20-35 mg/ml), was divided into multiple aliquots, snap-frozen in liquid nitrogen, and stored at −80° C. For experiments, the cytosol was thawed in a 37° C. water bath and on ice for use.

Isolation of Microsomal Membranes from H1299 cells for ER-budding Assay. H1299 cells were washed and scraped into 2 ml of ice-cold DPBS with protease inhibitors from duplicate 15 cm dishes. The cells were centrifuged at 1000×g for 5 min at 4° C. The tubes were snap-frozen in liquid nitrogen and stored at −80° C. after aspiration of the supernatants. When needed, the tubes were thawed in a 37° C. water bath for 50 sec and placed on ice. Each cell pellet was resuspended in 0.4 ml of Buffer B (10 mM HEPES-KOH (pH 7.2), 250 mM sorbitol, 10 mM KOAc, 1.5 mM Mg(OAc)₂, and protease inhibitors), passed through a 22-gauge needle 20 times, and centrifuged at 1000×g for 5 min at 4° C. The supernatants were transferred to siliconized microcentrifuge tubes (#1212M66, Thomas scientific) and centrifuged at 16,000×g for 3 min at 4° C. Subsequently, each pellet was resuspended in 0.5 ml of Buffer A and centrifuged again at 16,000×g for 3 min at 4° C. The microsomes for use in the in vitro vesicle-formation assay were obtained by dissolving the remaining pellet into 60-100 μl of Buffer A. The protein concentration was determined after a 5 μl of the microsomal suspension was added to 5 μl of a solution of 20% (w/v) of hexyl-β-D-glucopyranoside.

In Vitro Vesicle-Formation Assay. Each reaction in a final volume of 80 μl contained 50 mM HEPES-KOH at pH 7.2, 250 mM sorbitol, 70 mM KOAc, 5 mM potassium EGTA, 2.5 mM Mg(OAc)2, 1.5 mM ATP, 0.5 mM GTP, 10 mM creatine phosphate, 4 units/ml of creatine kinase, protease inhibitors, 37-80 μg protein of H1299 microsomes, and 600 μg of rat liver cytosol. Reactions were carried out in siliconized 1.5 ml microcentrifuge tubes for 15 min at 37° C., terminated by transfer of the tubes to ice, and then followed by centrifugation at 16,000×g for 3 min at 4° C. to obtain a medium-speed pellet (the membrane fractions) and a medium-speed supernatant. The medium-speed supernatants were collected from each sample and centrifuged again at 61,000 rpm for 40 min at 4° C. in a Beckman TLA120.1 rotor to obtain a high-speed pellet (vesicle fractions). The vesicle and membrane fractions were each resuspended in 60 μl of the buffer (10 mM Tris-HCl at pH 7.6, 100 mM NaCl, 1% (w/v) SDS plus protease inhibitors, supplemented with 15 μl of the buffer: 150 mM Tris-HCl at pH 6.8, 15% SDS, 25% (v/v) glycerol, 0.02% (w/v) bromophenol blue, and 12.5% (v/v) 2-mercaptoethanol) and heated at 100° C. for 10 min. The vesicle and membrane fractions were subjected to 10% SDS-PAGE and analyzed by immunoblotting.

Lipid Synthesis Assay. Cells were grown in serum-free media (containing 5 mM glucose and 2 mM glutamine) for 24 hr, which was then replaced with fresh serum-free media containing 5 mM glucose and stimulated with or without glutamine (4 mM) for 10 hr. After switching to new serum-free media (containing 2 mM glucose alone or together with 2 mM glutamine), 0.5 μCi 14C-glucose was added to media and incubated for 2 hr. The cells were washed twice with PBS and lipids were extracted with 500 μl hexane:isopropanol (3:1) for 1 hr. The liquid phase was collected in 1.5 ml tube left overnight to air-dry, and lipids were then dissolved in 200 μl chloroform for 0.5-1 hr before analysis with a scintillation counter (Beckman coulter).

Xenograft Mouse Models. Athymic nu/nu female mice (6-8 weeks old) were used. For lung cancer model, H1299-luc cells were transfected with pC3.0-GFP, pC3.0-GFP-SCAP wild-type or pC3.0-GFP-SCAP D428A for 24 hr. The cells were selected with 600 ng/ml G418 for two weeks and implanted into mice via tail-vein injection (1×106 cells/mouse suspended in 0.1 ml of PBS). After seven weeks, the mice were sacrificed, and the lungs were collected, fixed with 4% formaldehyde, and embedded in paraffin. Sections (5 μm) were cut and stained with H&E and IHC. For intracranial xenograft models, GBM30 cells stably expressing GFP, GFP-SCAP wild-type, or GFP-SCAP D428A mutant (3.5×103 cells in 4 μl of PBS) were stereotactically implanted into mouse brain. Mice were observed and scanned by Magnetic Resonance Imaging (MRI) until they became moribund, at which point they were sacrificed. All animal procedures were approved by the Subcommittee on Research Animal Care at Ohio State University Medical Center.

Hematoxylin and Eosin Staining. Paraffin tissue sections were deparaffinized in xylene and rehydrated in degrading ethanol dilutions (100%, 95% and 70% ethanol). After washing with dH2O, slides were stained with hematoxylin and eosin (H&E) solution in sequence, followed by washing with dH2O. Slides were then dehydrated in degraded ethanol and immersed in xylene, followed by mounting in Permount (VECTOR, #H-5000-60).

Mouse Luminescence Imaging. Mice implanted with H1299 cells expressing luciferase were intraperitoneally injected with a Luciferin (#122796, Perkin Elmer) solution (15 mg/ml in PBS, dose of 150 mg/kg). The bioluminescence images were acquired using the IVIS Lumina system and analyzed by the Living Image software.

Magnetic Resonance Imaging. Animals were anesthetized with 2.5% isoflurane mixed with 1 L/min carbogen (95% 02 with 5% CO2) then maintained with 1% isoflurane. Physiological parameters, including respiration and temperature, were monitored using a small animal monitoring system (Model 1025, Small Animals Instruments, Inc. Stony Brook, NY). A pneumatic pillow was used to monitor respiration. Core temperature was maintained using circulating warm water within the animal holder. Animals were injected intraperitoneally with 0.1 M gadolinium-based contrast agent used at a ratio of 100 μl per 25 g body weight. Imaging was performed using a Bruker BioSpec 94/30USR MRI system (Bruker BioSpin, Karlsruhe, Germany) and a mouse brain circularly polarized (CP) surface coil and an 86 mm diameter CP volume coil as receiver and transceiver coils, respectively. Data were collected using a T1-weighted RARE sequence with the following acquisition parameters: TR=1200 ms, TE=7.5 ms, Rare factor=4, NA=3, FOV=20 mm×20 mm, matrix size=256×256, slice thickness=1 mm, number of slices=18. A T2-weighted RARE sequence was also used following T1-weighted acquisition (parameters: TR=2500 ms, TE=33 ms, Rare factor=8, NA=4, FOV=20 mm×20 mm, matrix size=256×256, slice thickness=1 mm, number of slices=18). For data analysis, a region-of-interest (ROI) that included tumors (hyper-intense regions) was outlined. Tumor volumes were calculated from the outlined ROIs. All imaging experiments were conducted at the OSU Small Animal Imaging Core.

Molecular Dynamics Simulations. The cryo-EM structure of the Insig/SCAP complex (PDB ID: 6M49)50 was used as the initial structure for our simulations. The SCAP structure without 25-HC was prepared by replacing the partially unfolded S4 helix (residues 354-358) in the inactive conformation with a fully folded S4 helix, which was built with Modeller V10.1 using NPC1 (PDB code: 6W5S)67 as a template. The CHARMM-GUI membrane builder was used to build a membrane bilayer consisting of 366 hydrated palmitoyl-oleyl-phosphatidylcholine (POPC) molecules68,69. Each system was solvated with approximately 34,000 TIP3P water molecules (a type of water used in simulations that represents 3-site rigid water molecule with charges and Lennard-Jones parameters assigned to each of the 3 atoms (HOH)) and 0.15 M NaCl70. The CHARMM 36 force field was used for the proteins, lipids, and ions, while the ligand (25-HC) was parameterized using SwissParam71. All simulations were performed at 310K and the temperature was regulated with the v-rescale scheme 72. The solutes (protein, membrane, and ligand) and solvents (water and ions) were coupled separately with a relaxation time constant of 0.1 ps. The Parrinello-Rahman barostat was used to keep the pressure at 1 bar with a coupling constant of 0.2 ps. The isothermal compressibility was 4.5×10-5 bar-1. The pressure was coupled semi-isotropically, where the x and y directions were coupled together, and the z direction was independently coupled. All bonds were constrained with the LINCS algorithm. The integration time step was 2 fs. The non-bonded long-range electrostatic interactions were calculated using the particle mesh Ewald method with a 14 Å cutoff. The van der Waals interaction also used a 14 Å cutoff. All simulations were carried out using Gromacs 202073. Each system was first energy minimized with the steepest-descent method with a maximum of 50,000 steps or the maximum force in the system reaching less than 100 kJ/mol-1 Å-2. After energy minimization, a 500 ps equilibration simulation was performed with position restraints on the protein, lipids, and ligands, which was followed by six 1 ns simulations with decreasing position restraints. Finally, one ˜1 μs-long production simulation without any restraints was run for each system, with trajectories saved every 100 ps (a total of ˜10,000 frames for each simulation) for subsequent analysis.

Quantification and Statistical Analysis. All figures, including western blots, metabolites analysis, and mouse experiments, are representative of at least two biological replicates, unless stated otherwise. Data analysis was performed using GraphPad Prism 7. Statistical significance was obtained using paired or unpaired Student's t test, or one-way or two-way ANOVA depending on the data. Multiplicities were adjusted by the Dunnetts's or Turkey methods. Kaplan-Meier method was used to generate patient and mice overall survival curves and the difference in survivals was tested by Log-rank test.

### Results

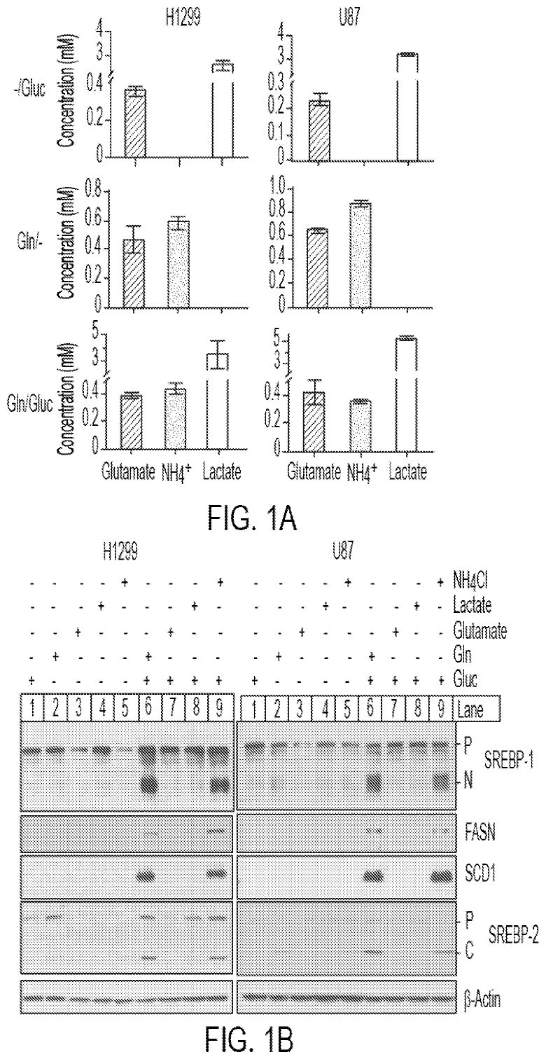

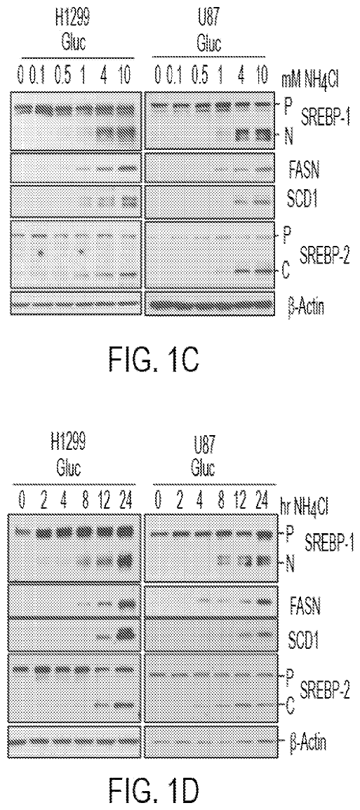

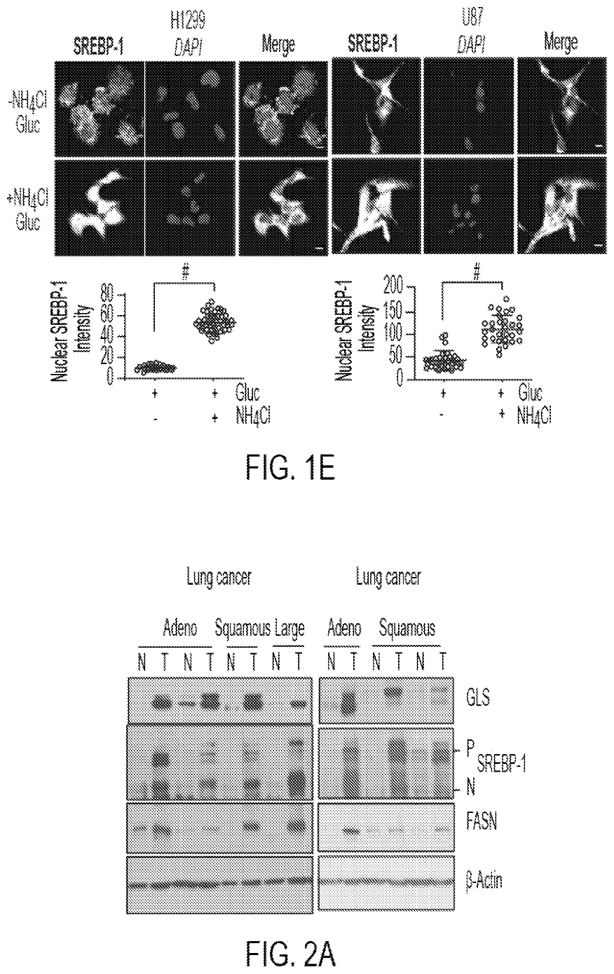

Glutamine-released ammonia is a key activator for SREBP activation and lipogenesis. Glutaminolysis is known to be highly activated in many cancers to promote rapid growth. In this process, glutamine is first deaminated by glutaminase (GLS) to release the polar molecule, ammonia (NH₃), and produce glutamate. Glutamate is further converted to α-ketoglutarate (α-KG) that incorporates into the tricarboxylic acid (TCA) cycle in the mitochondria for energy production. Using the Bioprofile 100 Plus Analyzer, the levels of the major metabolites derived from glutaminolysis, and glycolysis were measured in the culture medium of H1299 and U87 cells. NH3, which is converted to NH₄+ in aqueous solution, and glutamate, were detected in the media from cells cultured in the presence of glutamine without glucose (12 hr) (FIG. TA, middle panels). NH₃ and NH₄+ are thereafter referred as ammonia. In contrast, when cells were cultured in the presence of glucose but without glutamine, lactate and glutamate were detected (FIG. 1A, top panels), indicating that glutamate could be derived from glucose when glutamine is absent. When combining glutamine and glucose, all three metabolites, i.e., NH₄+/glutamate/lactate, were detected in the culture medium (FIG. 1A, bottom panels).

Next, metabolites involved in SREBP activation were examined. Western blot analysis showed that neither glutamate, ammonia (derived from added NH₄Cl) or lactate alone, nor the combination of glucose with glutamate or lactate were able to activate SREBP-1 or -2 cleavage and promote FASN and SCD1 expression (FIG. 1B). Interestingly, in the presence of glucose, ammonia induced SREBP-1 and -2 cleavage, and FASN and SCD1 expression to a similar extent as the combination of glutamine and glucose did (FIG. 1B, lane 9 vs. lane 6). Moreover, the effects of ammonia were dose- and time-dependent (FIG. 1C and FIG. 1D). Immunofluorescence imaging further showed that in the presence of glucose, ammonia markedly stimulated SREBP-1 translocation into the nucleus without the presence of glutamine (FIG. 1E), while glutamate, lactate or α-KG stimulation failed to do so.

Together, these data demonstrate that ammonia, released by glutamine, is a key activator of SREBP activation and lipogenesis, and requires the presence of glucose to maintain SCAP stability via its N-glycosylation, a prerequisite condition for SREBP activation by glutamine or ammonia.

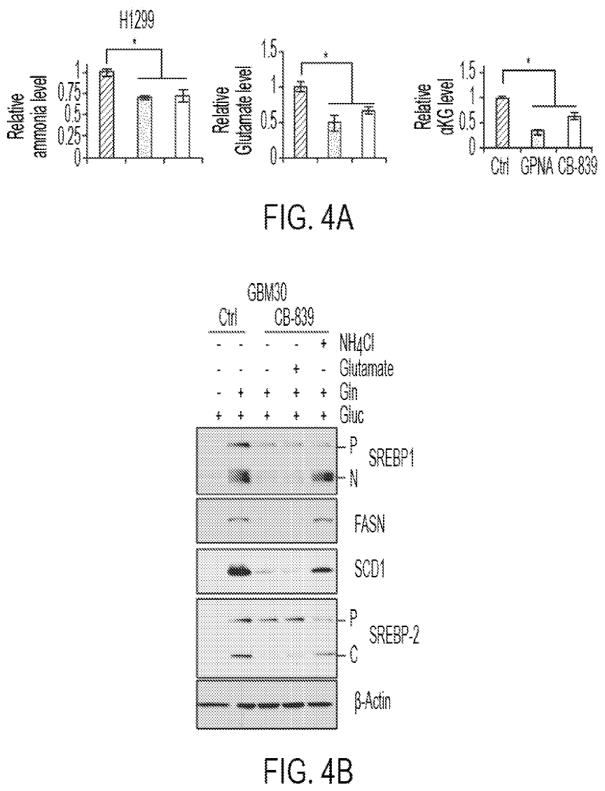

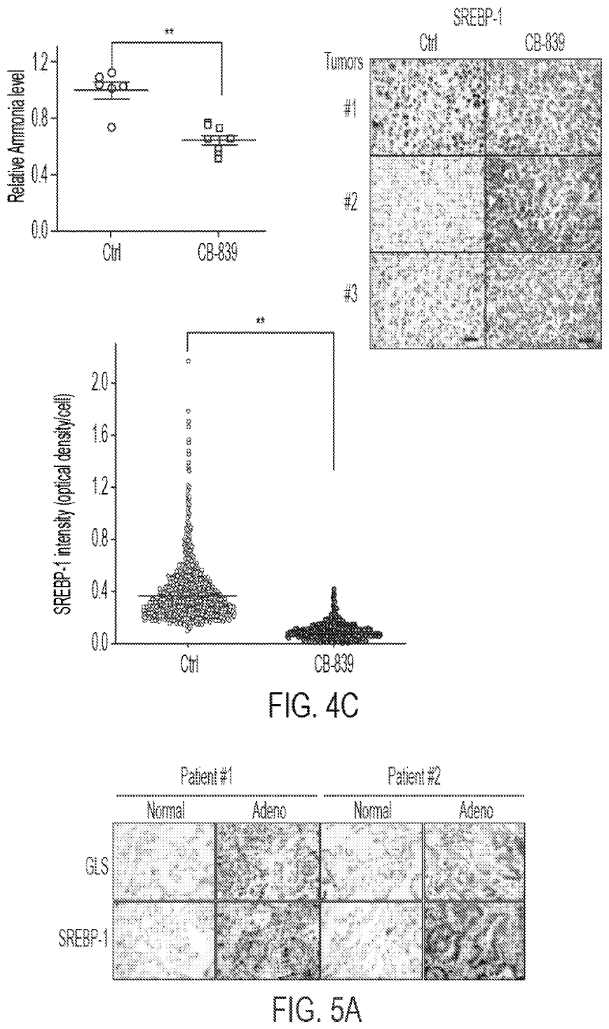

Suppressing ammonia release from glutamine by inhibiting GLS abolishes SREBP activation and lipogenesis. Next, the role of ammonia released from glutamine in the stimulation of SREBP activation and lipogenesis by inhibiting glutamine uptake and glutaminolysis was validated. Glutamine uptake was suppressed with γ-glutamyl-p-nitroanilide (GPNA), an inhibitor of SLC1A5 (also named ASCT2) that has been characterized as the major glutamine transporter, and blocked glutaminolysis with CB-839, a GLS inhibitor. Metabolite analysis showed that both GPNA and CB-839 treatment dramatically reduced the levels of glutamate, ammonia, and α-KG in cells and in cell culture media (FIG. 4A), which was associated with a significant reduction of glutamine consumption by both H1299 and U87 cells. Then, cells were supplemented with ammonia (by adding NH4Cl), glutamate or α-KG to determine which metabolite was able to restore SREBP activation inhibited by CB-839. Western blot analysis clearly showed that only ammonia, and not glutamate or α-KG, was able to strongly restore SREBP activation and FASN/SCD1 expression (FIG. 4B). It was further examined whether the effects of inhibition of GLS by CB-839 on SREBP-1 activation in an H1299-derived xenograft mouse model. Consistent with the reduction of ammonia levels in tumor tissues (FIG. 4C, left panel), immunohistochemistry (IHC) staining showed that SREBP-1 levels were significantly reduced in tumors from mice treated with CB-839 as compared to the vehicle treatment group (FIG. 4C, right panel). GLS was also genetically inhibited using lentivirus-mediated shRNA to suppress the release of ammonia from glutamine.

Together, these data confirm that ammonia is released from glutamine to activate SREBPs and lipogenesis in concert with glucose, unveiling anew glutamine-GLS-ammonia-SREBP activation axis that links glutaminolysis and lipogenesis, two highly upregulated metabolic pathways in various types of cancers.

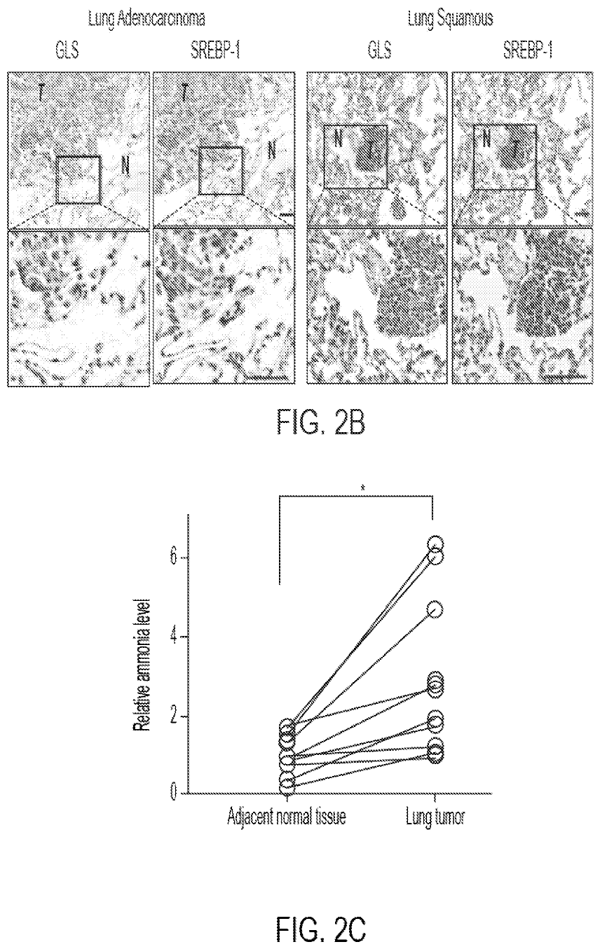

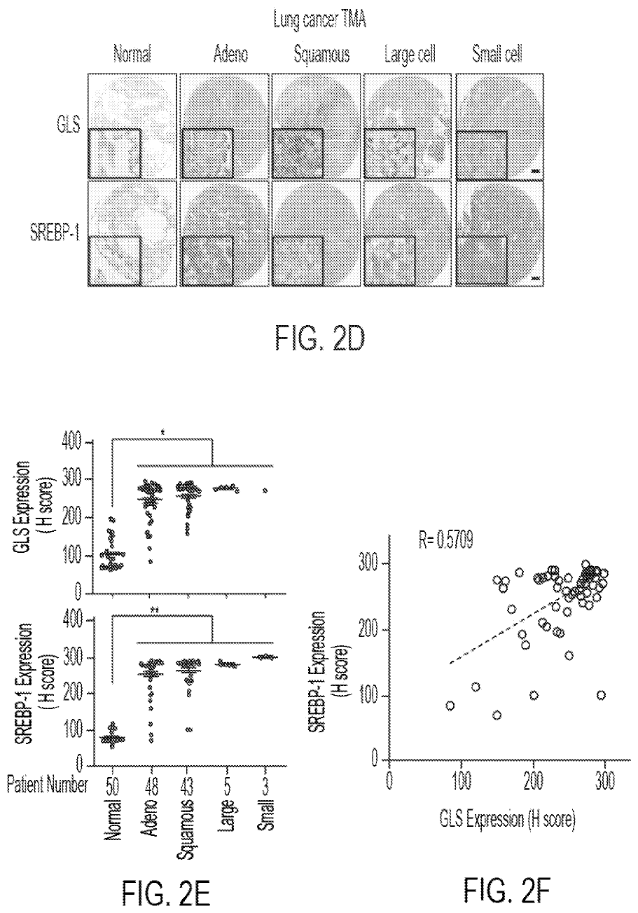

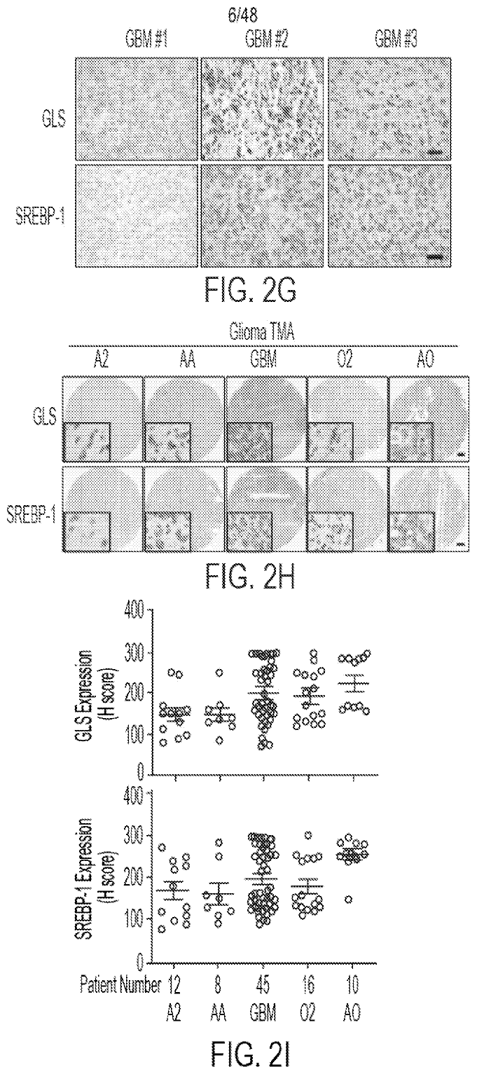

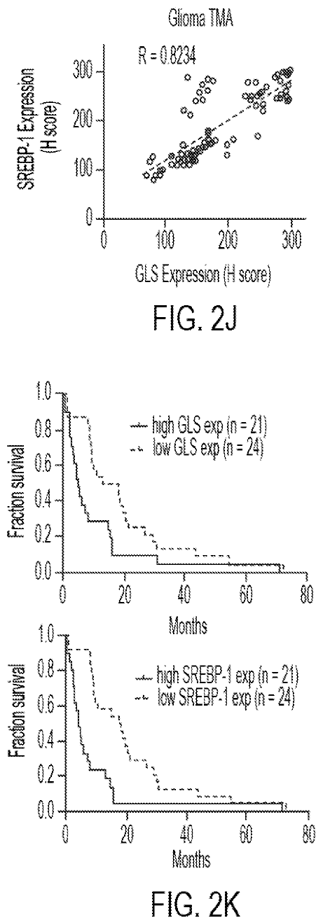

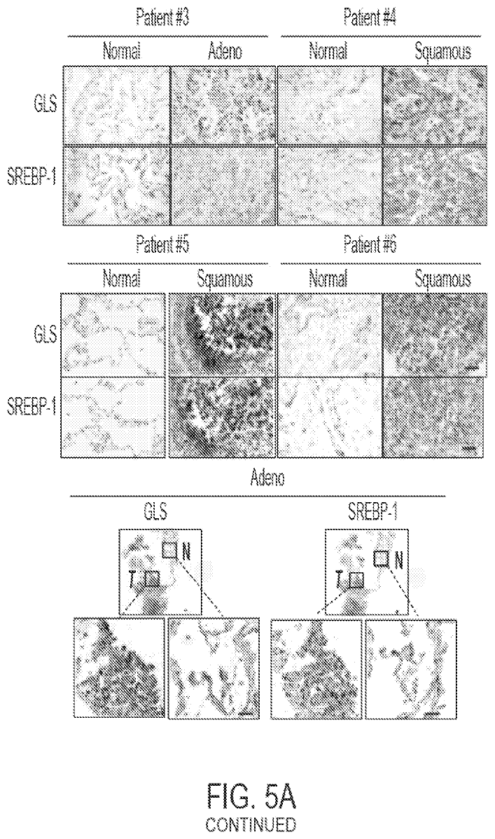

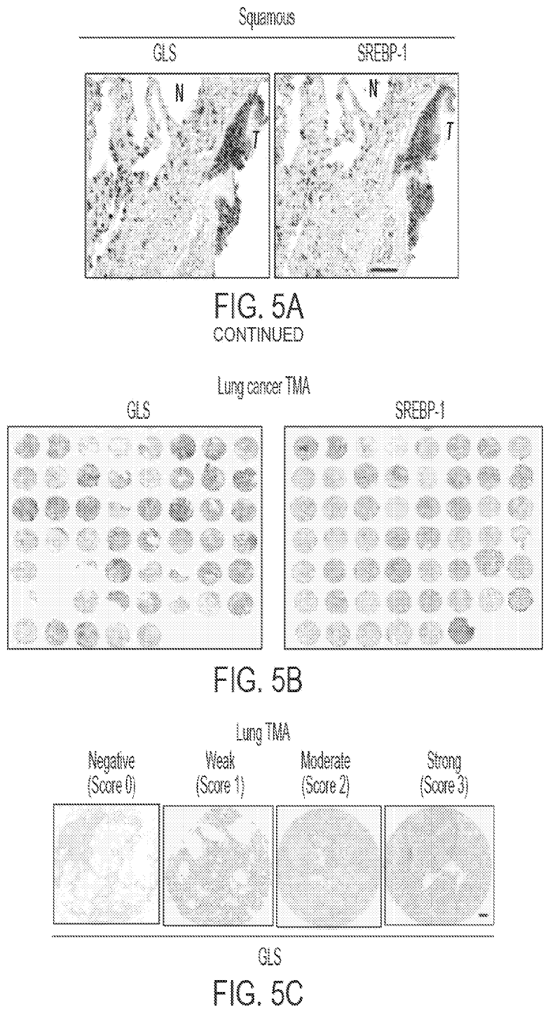

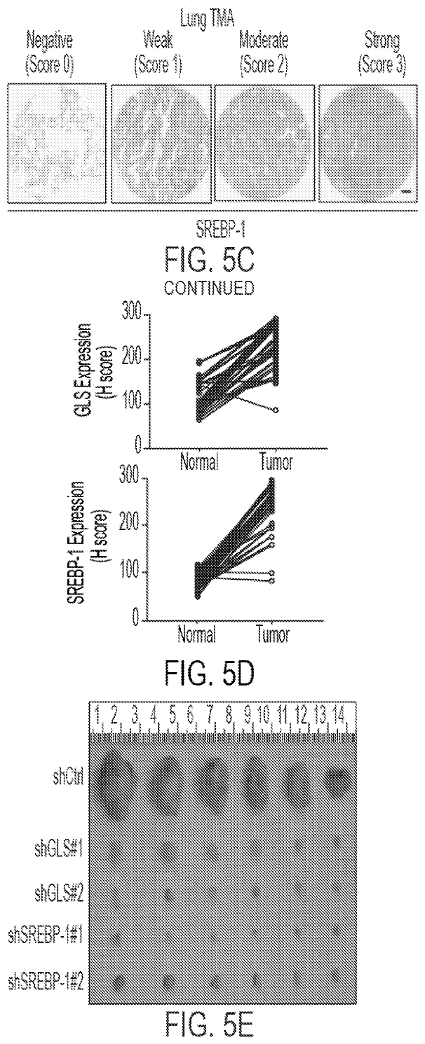

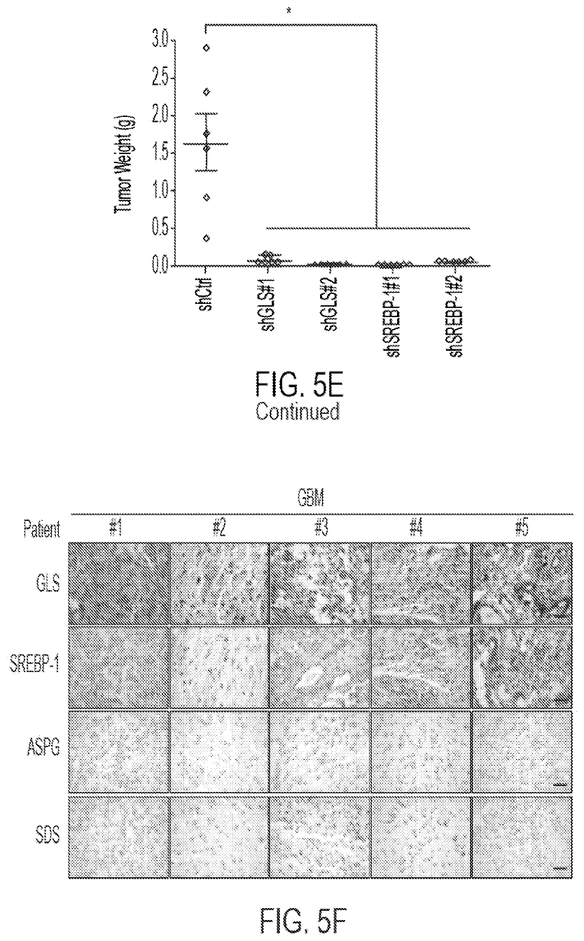

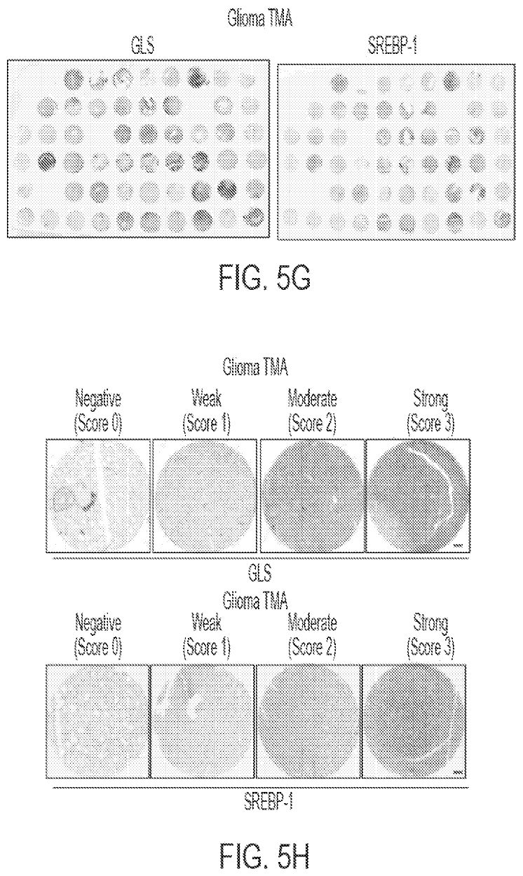

High GLS expression is significantly correlated with strong SREBP-1 activation in lung cancer and glioma clinical samples. We next examined whether the molecular connection between glutaminolysis and lipogenesis identified above could be validated in human tissues. Seven paired frozen tissues, i.e., tumors vs. adjacent normal human lung tissues, were measured from individuals with adenocarcinoma (Adeno), squamous, and large cell lung cancer by western blot. The data showed that all seven tumor tissues contained high levels of GLS, and strong SREBP-1 expression and cleavage, together with dramatically increased FASN protein in comparison with adjacent normal lung tissues (FIG. 2A). Then, multiple paraffin-embedded tumor vs. adjacent normal lung tissues were examined from individuals with adenocarcinoma and squamous lung cancer by IHC staining. Consistent with the western blot results, IHC staining showed that GLS expression, and cytoplasmic and nuclear SREBP-1 staining were highly elevated in tumor tissues (T), while both were low in adjacent normal tissues (N) (FIG. 2B and FIG. 5A). The ammonia levels were then measured in ten paired human lung tumors vs. adjacent normal lung tissues using an ammonia assay kit. Consistent with the elevation of GLS expression and SREBP-1 activation (FIG. 2A and FIG. 2B, FIG. 5A), the data showed that ammonia levels were significantly higher in tumors than in paired normal tissues (FIG. 2C). A tissue microarray (TMA) containing 99 tumors and 50 matched adjacent normal lung tissues were then examined from individuals with different types of lung cancer. IHC staining showed that over 90% of lung tumor tissues contained high level of GLS and strong SREBP-1 staining as compared to adjacent normal lung tissues (FIG. 2D and FIG. 2E, FIG. 5B-5D). Pearson correlation analysis showed that GLS expression was strongly correlated with SREBP-1 staining in these lung cancer tissues (FIG. 2F). Accordingly, genetic knockdown of GLS in a xenograft model gave the same result as SREBP-1 knockdown, dramatically suppressing tumor growth in H1299 cells-derived xenograft mouse model (FIG. 5E). It was also examined multiple GBM tissues and a TMA with 91 glioma samples, including low-grade astrocytoma (A2) and oligodendroglioma (O2) up to high-grade anaplastic astrocytoma (AA), GBM and anaplastic oligodendroglioma (AO). As in lung cancer tissues, IHC staining showed that high GLS expression and strong SREBP-1 staining were associated in tumor tissues across low to high grade gliomas (FIGS. 2G-2I, FIG. 5F-5H). Pearson correlation analysis showed that GLS expression was strongly correlated with SREBP-1 staining in these glioma tissues (FIG. 2J). Kaplan-Meier plot analysis further showed that higher GLS expression and stronger SREBP-1 staining were associated with poorer survival in individuals with GBM (FIG. 2K). The presence of other amino acid deaminases, specifically ASPG and SDS, were determined by IHC. Staining showed that neither enzyme was detected in GBM tumor samples (FIG. 5F), confirming the specific positive correlation between GLS and SREBP-1 in patient tissues. Together, these large clinical sample analyses demonstrate that GLS expression is significantly correlated with SREBP-1 activation in human cancers, which provides strong evidence in support of the molecular connection between glutaminolysis and lipogenesis under physiological conditions.

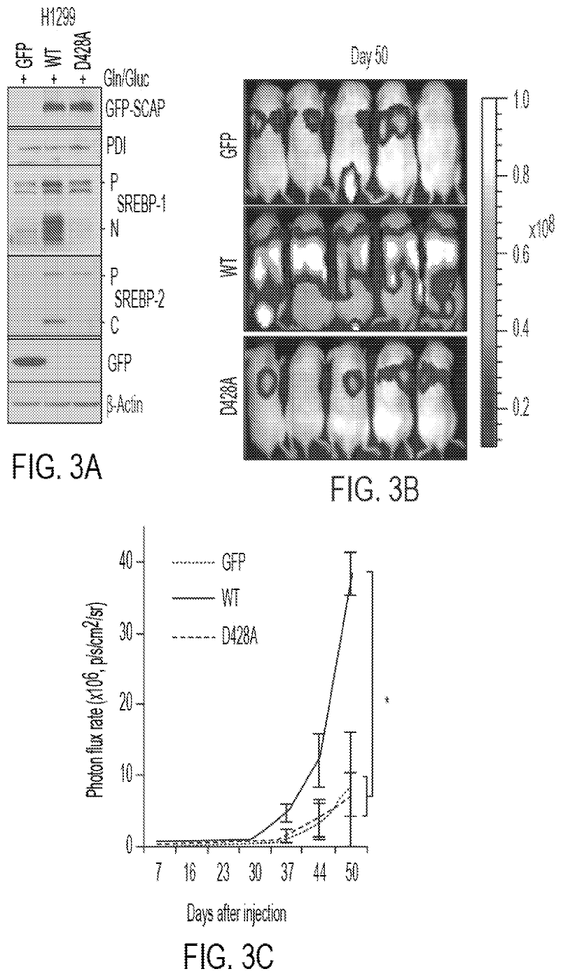

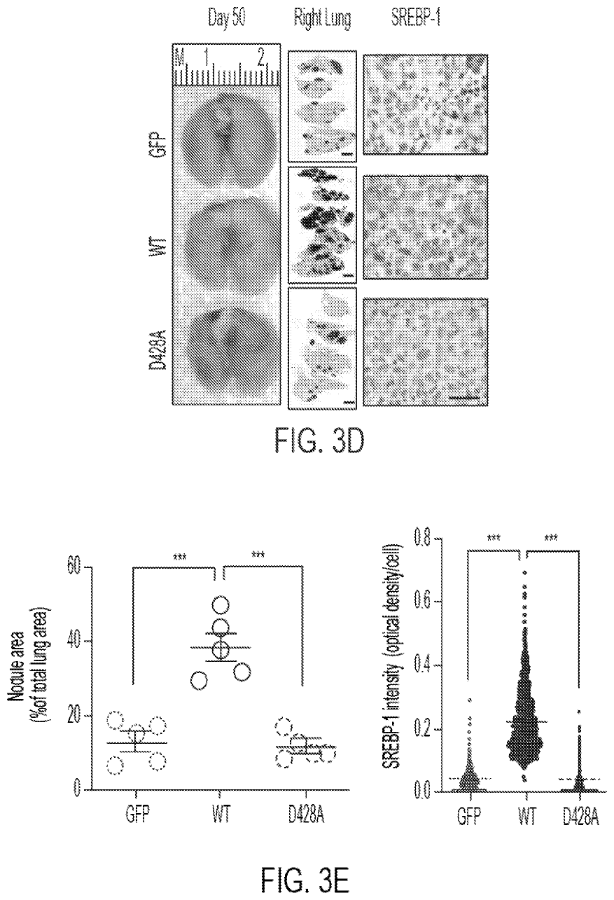

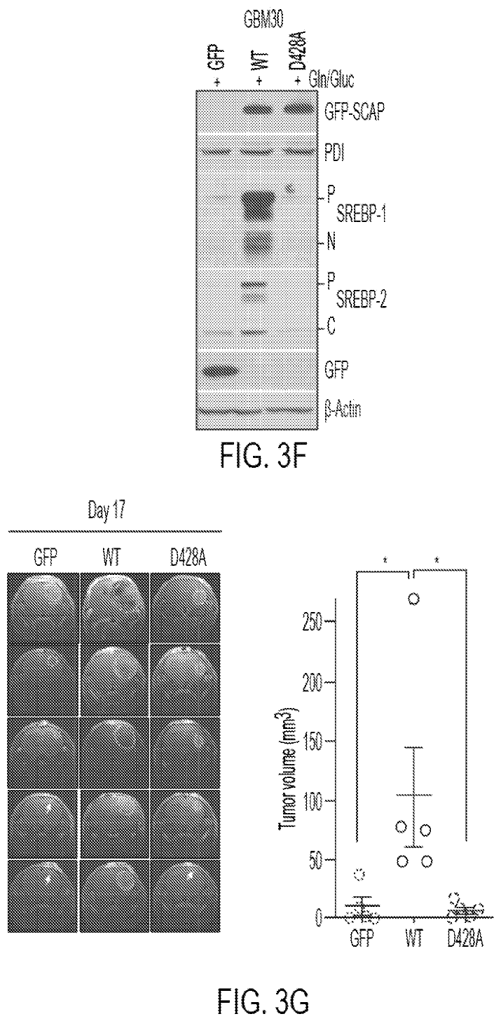

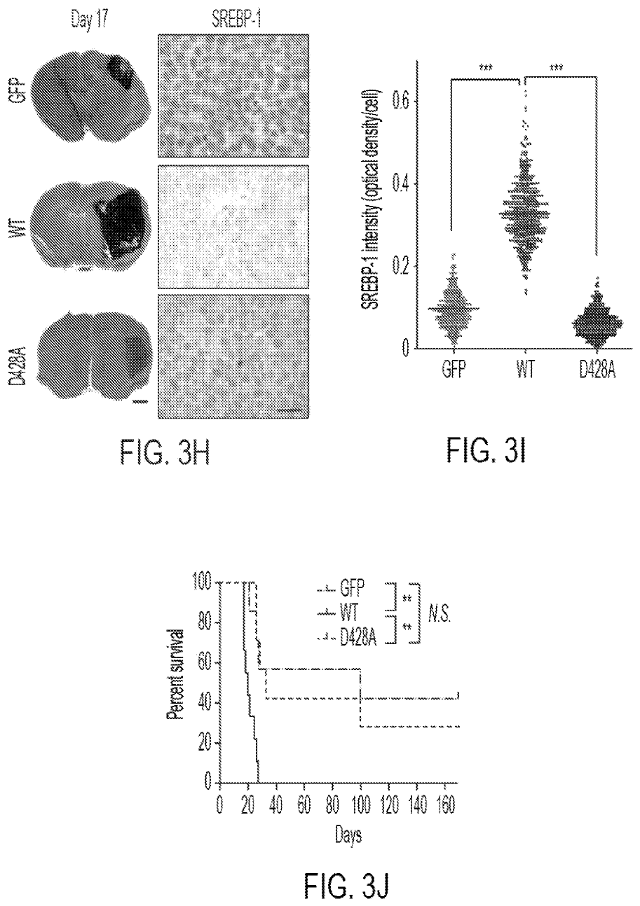

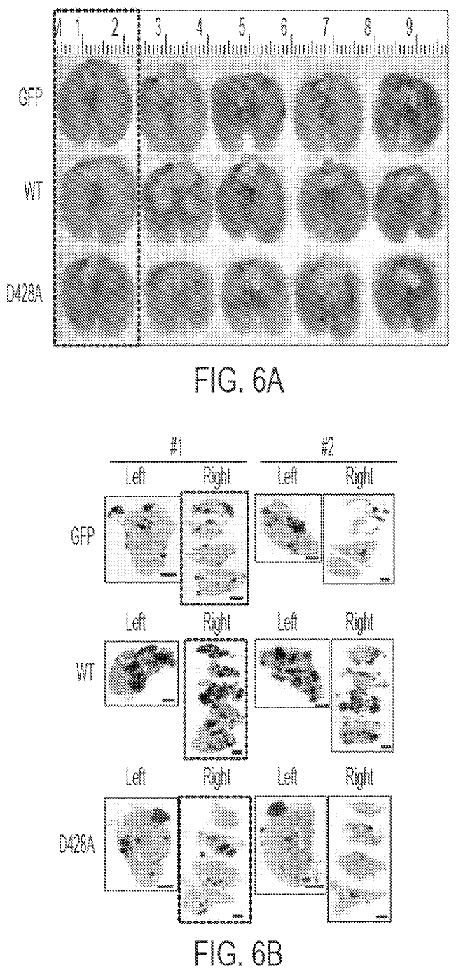

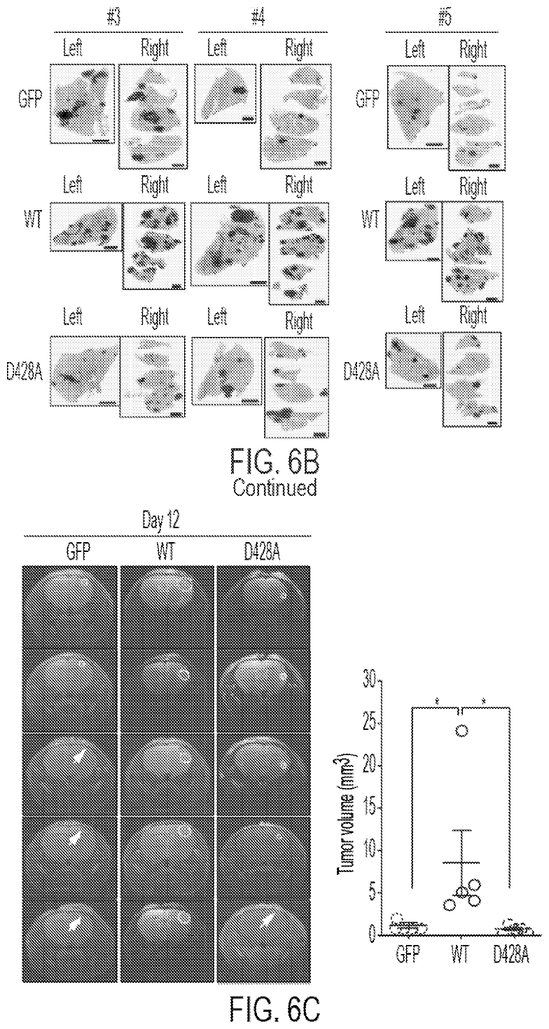

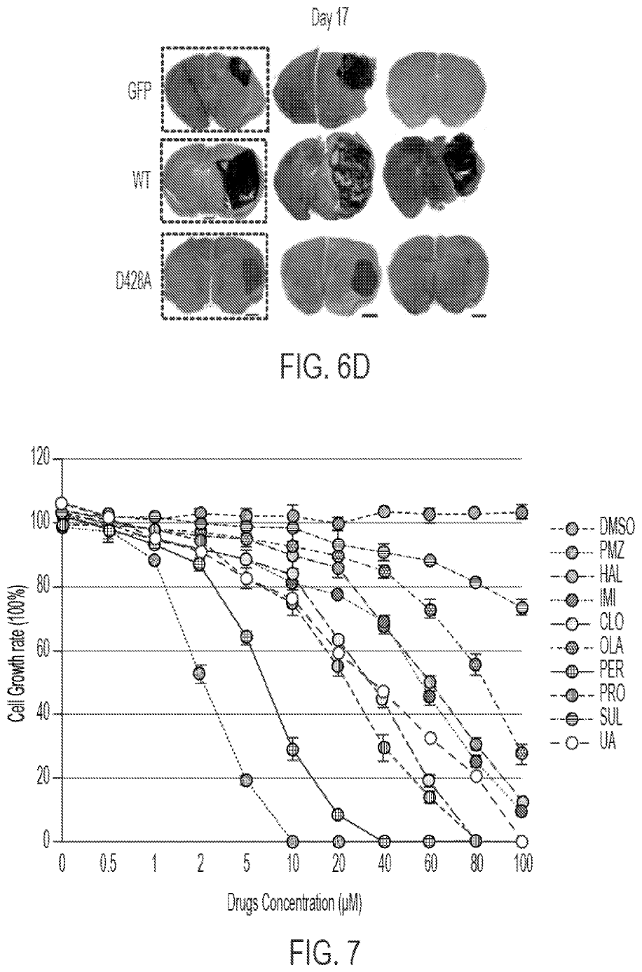

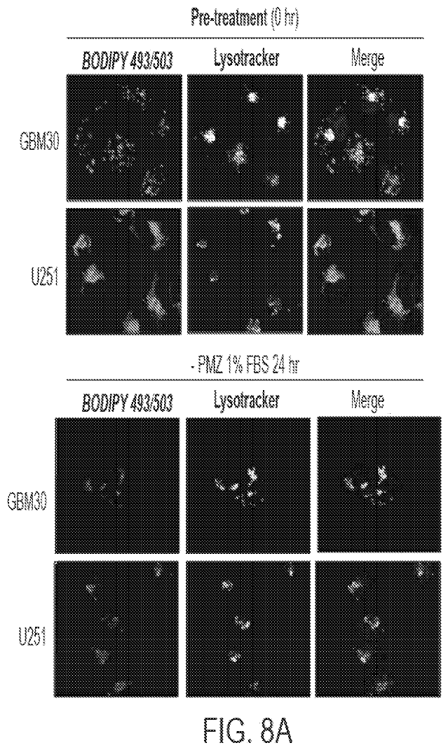

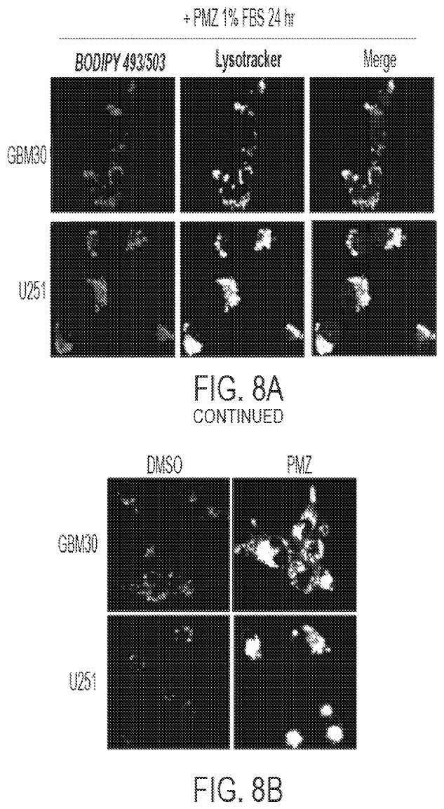

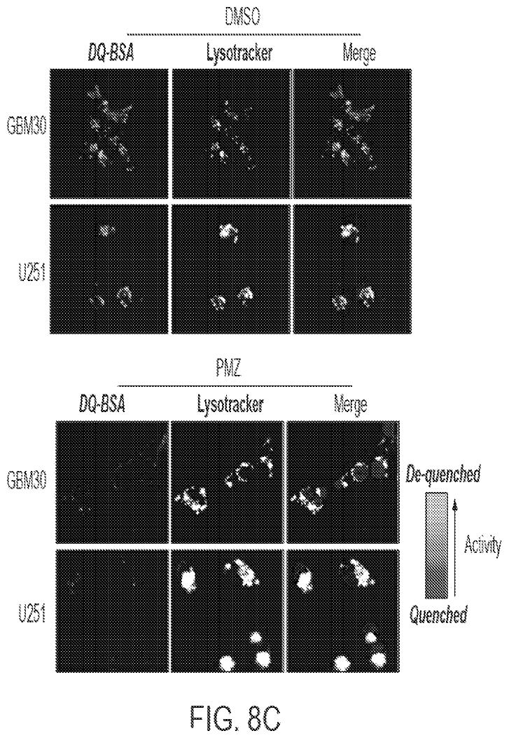

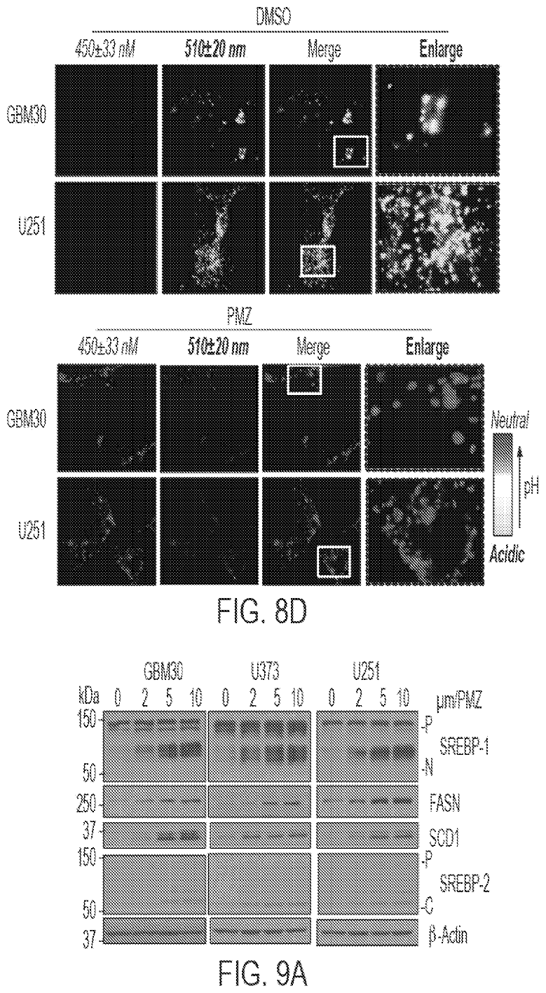

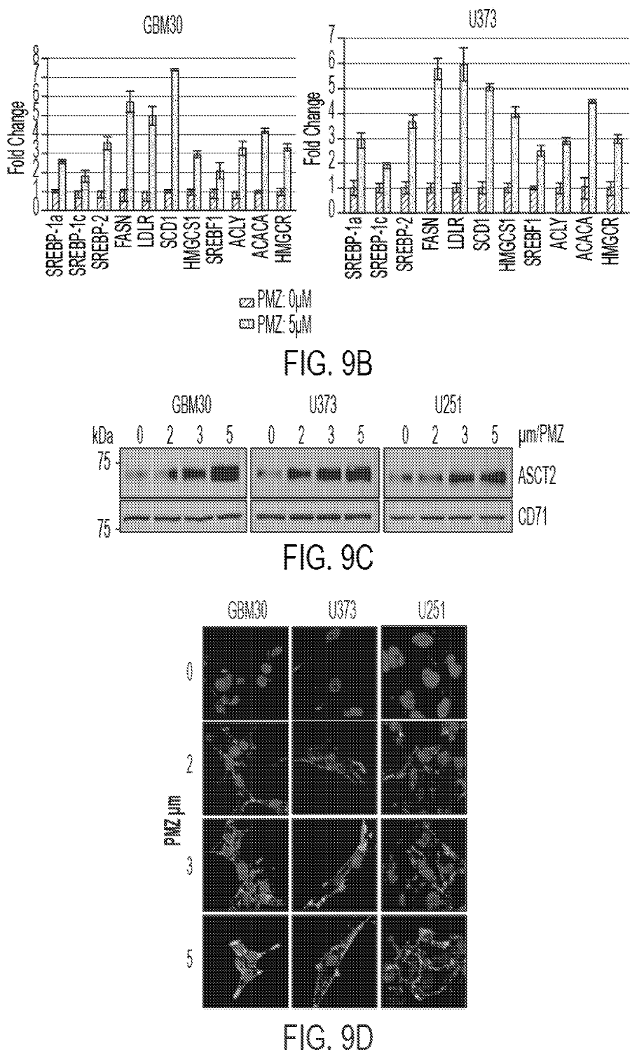

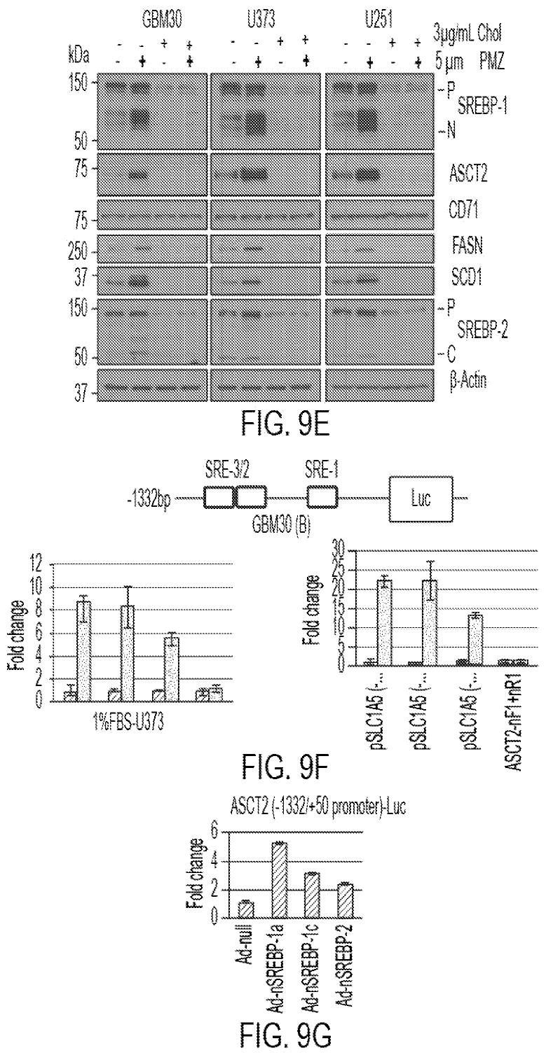

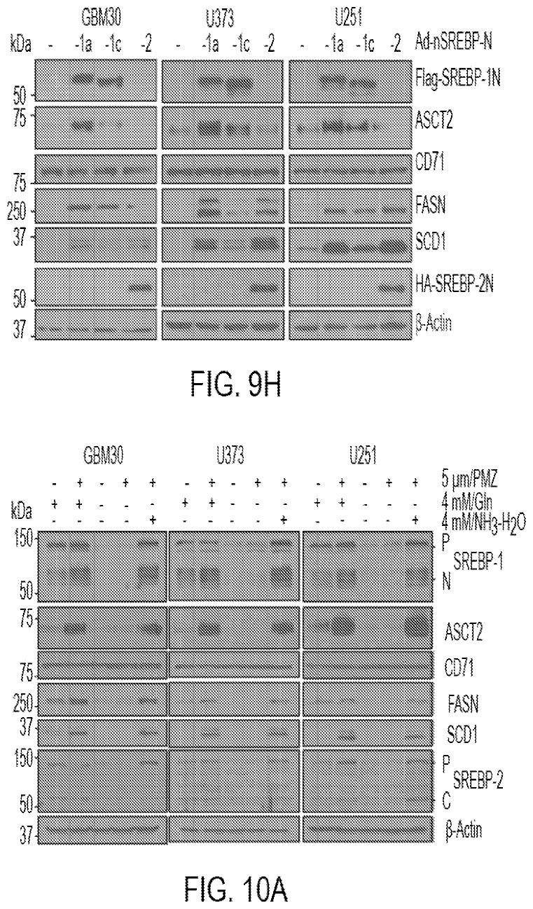

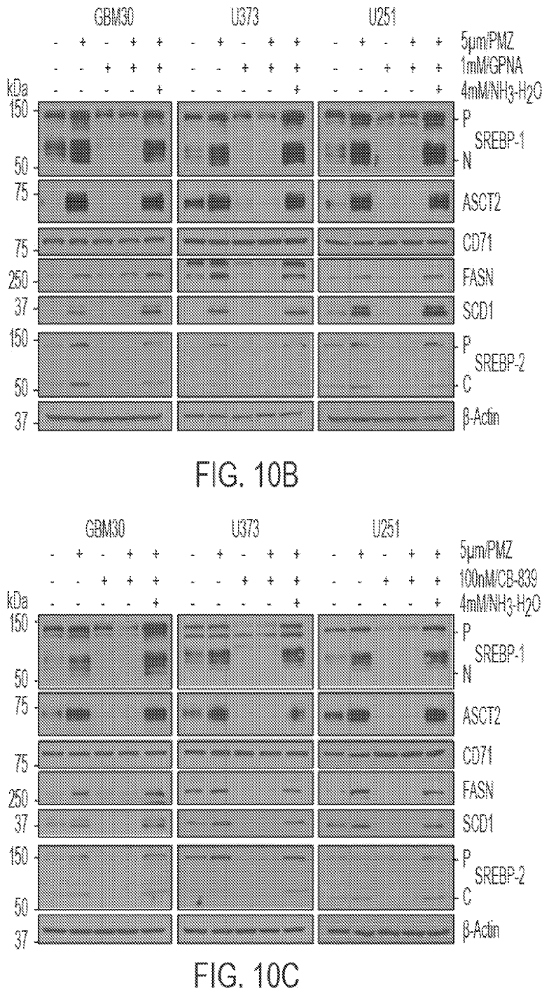

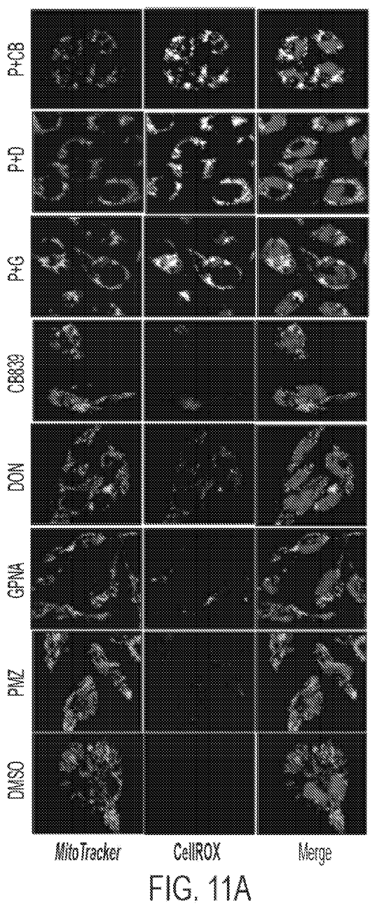

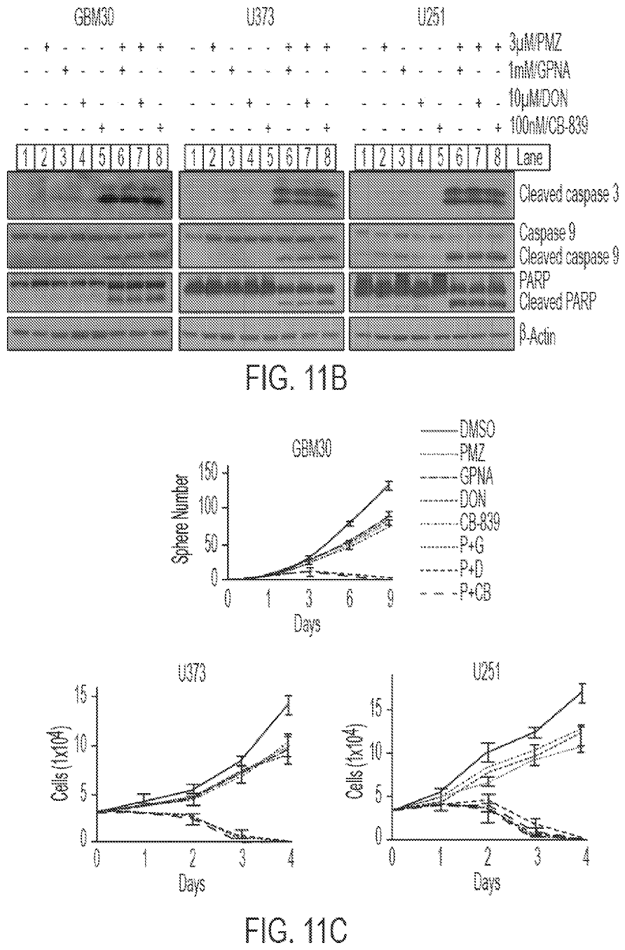

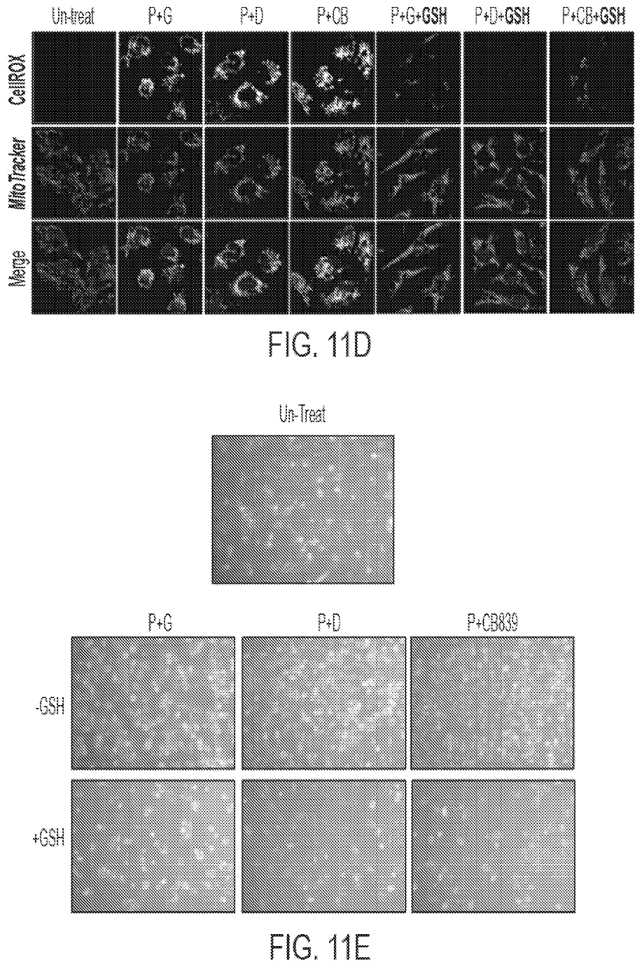

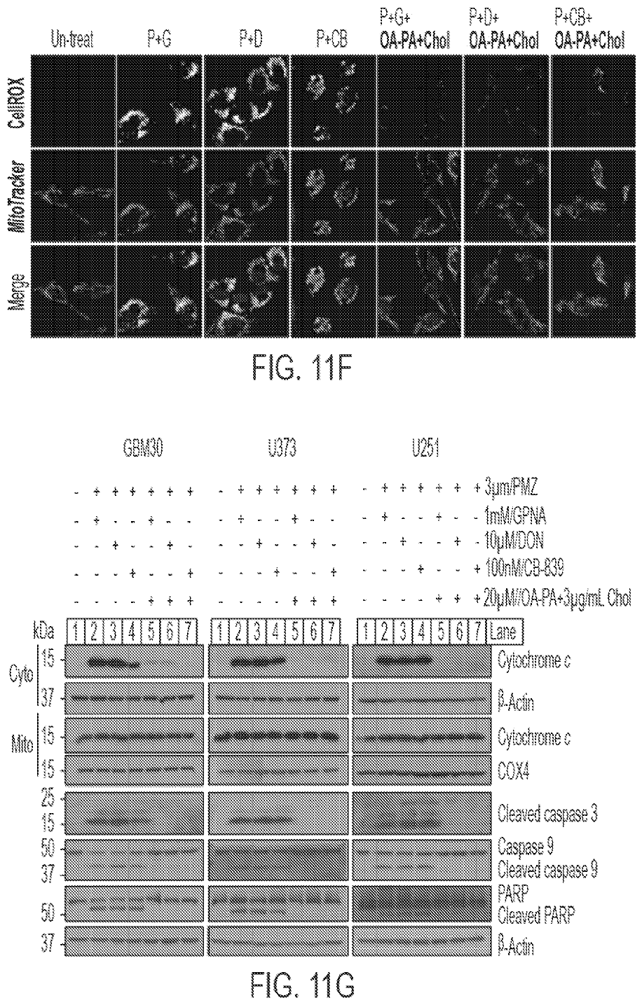

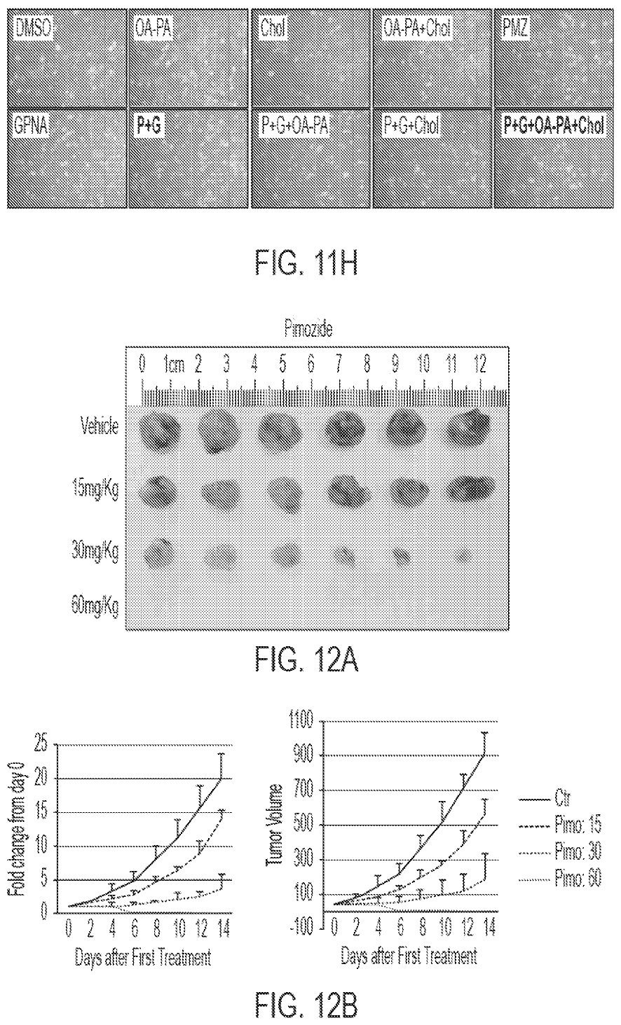

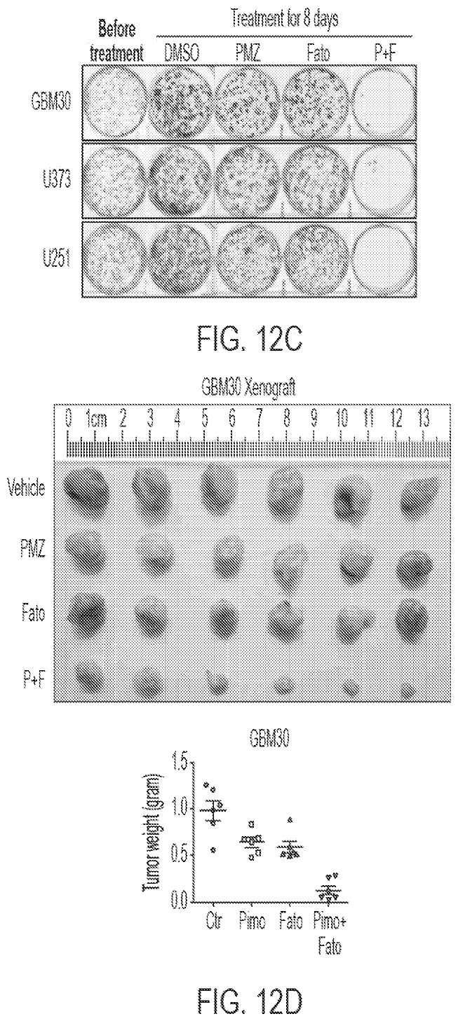

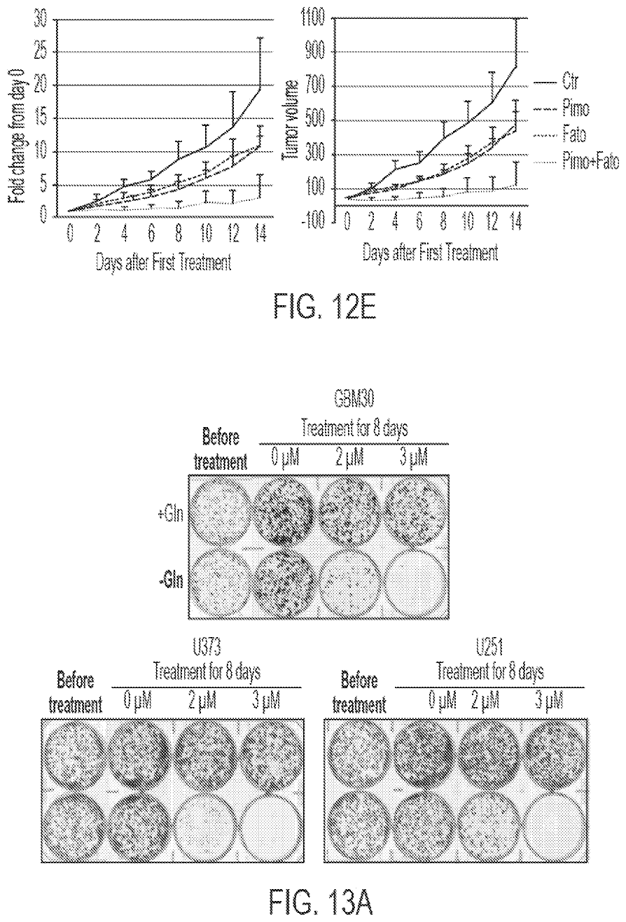

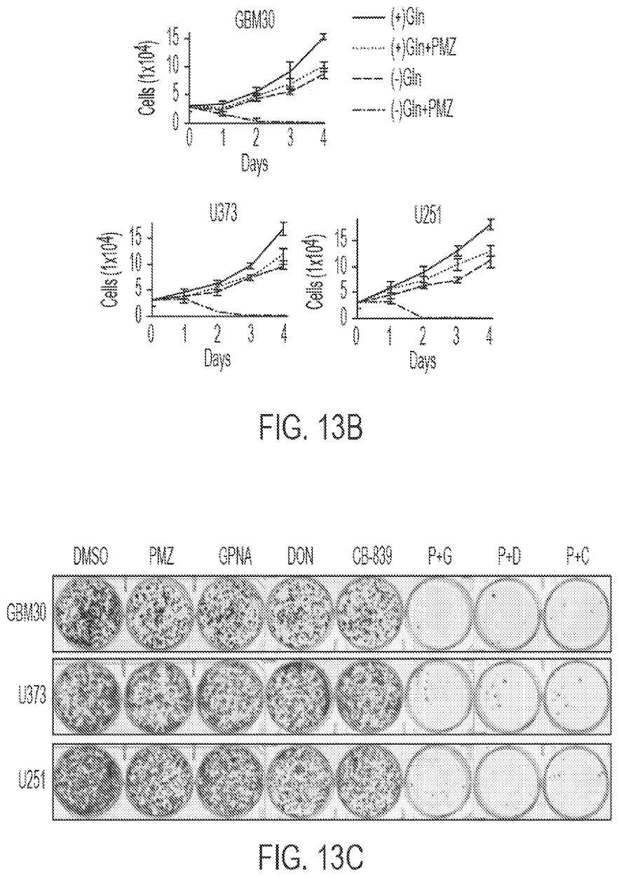

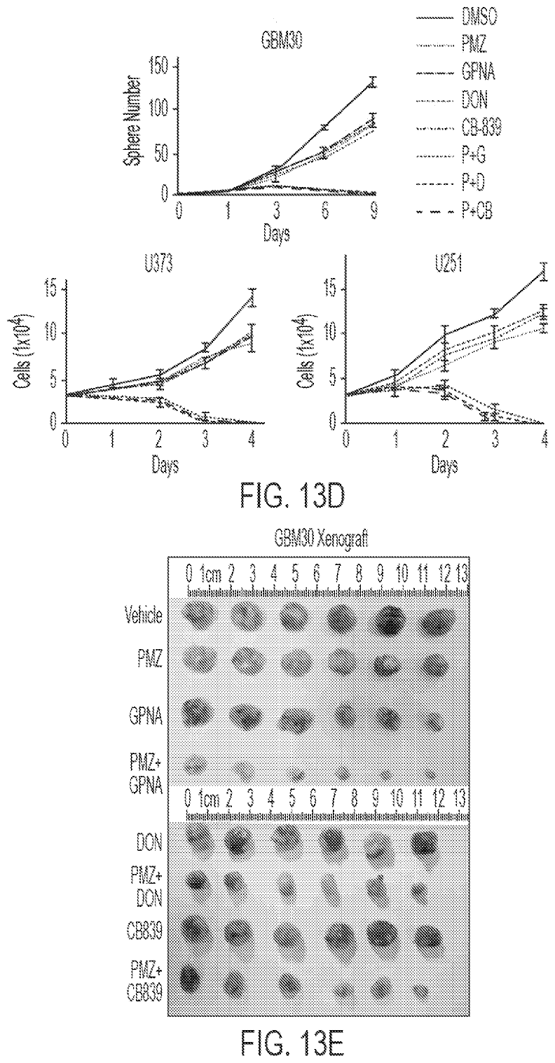

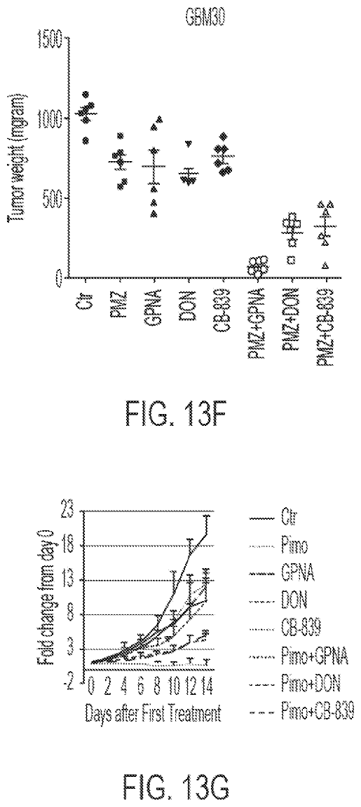

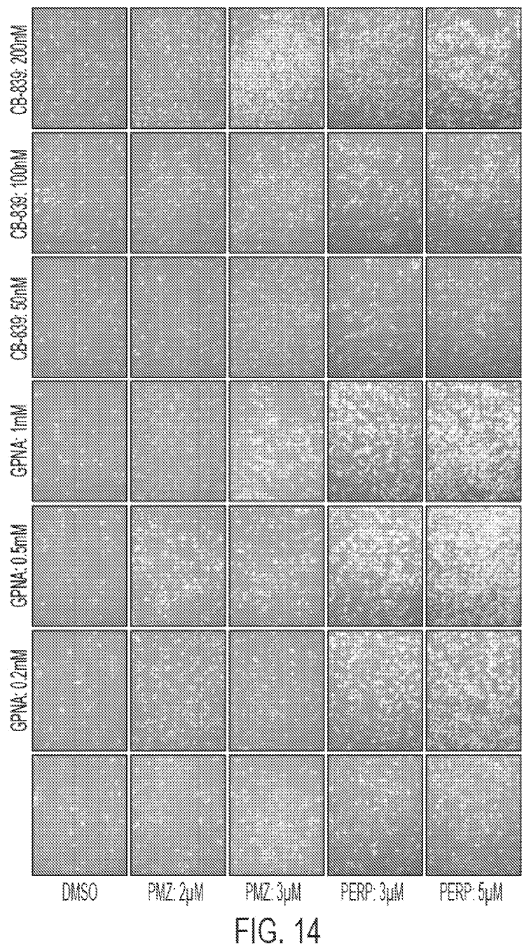

Disrupting SCAP interaction with ammonia via mutation of D428 suppresses lung cancer and glioblastoma growth. It was next examined whether disrupting SCAP-ammonia interaction by changing D428 to alanine (D428A) in SCAP could affect tumor growth. GFP, GFP-SCAP wild type or D428A mutant were transfected into H1299 lung cancer cells that stably express luciferase. Western blot analysis showed that wild-type SCAP expression dramatically increased SREBP-1 and -2 cleavage in the presence of both glucose and glutamine, which was abolished by the D428A mutation (FIG. 3A). These stably transfected cells were then implanted into mice via tail vein injection, and mice were examined by bioluminescence imaging, which clearly showed at day 50 post-implantation that wild-type SCAP expression dramatically increased tumor growth in the lung area as compared to the control GFP group, while the D428A mutation abolished this increase (FIG. 3B and FIG. 3C). Gross lung images showed higher numbers of tumor lesions on the lung surfaces in the wild-type SCAP group as compared with the GFP group, while the lungs in the D428A mutant group had dramatically less tumor lesions than those in the wild-type SCAP (FIG. 3D, left panels, FIG. 6A).

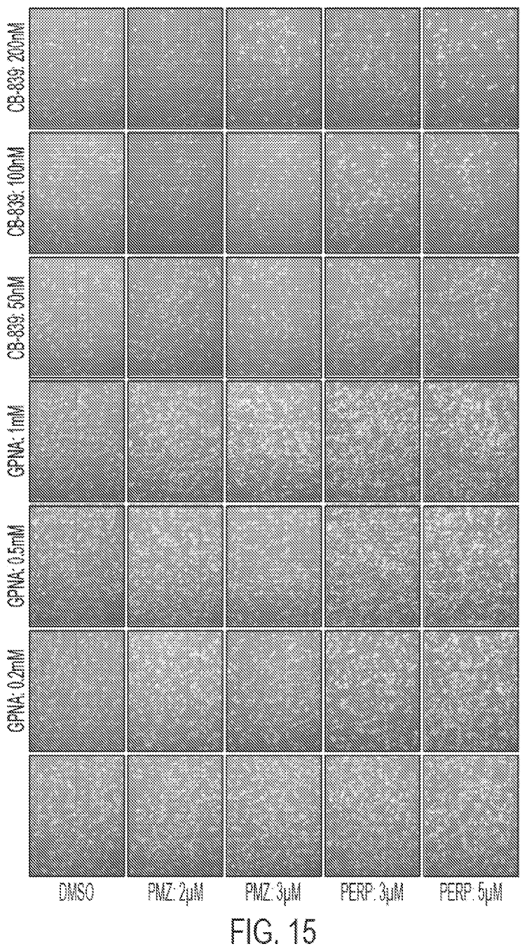

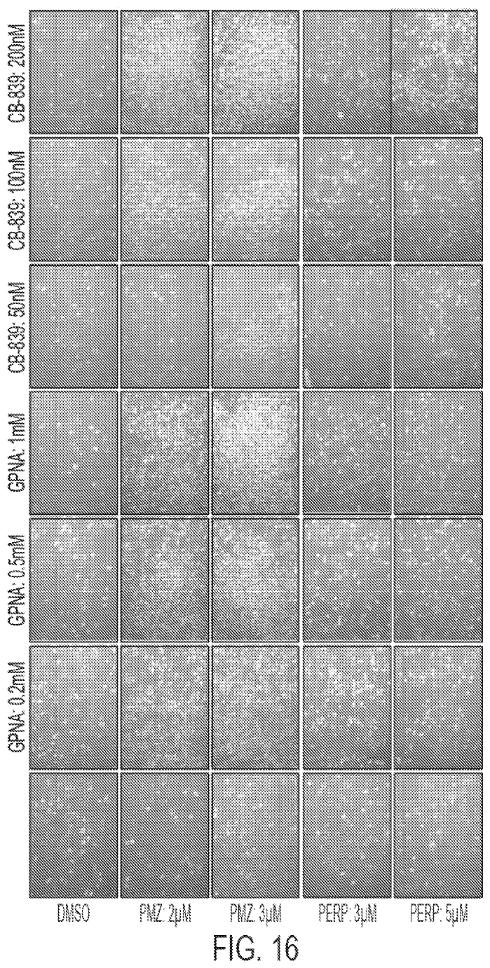

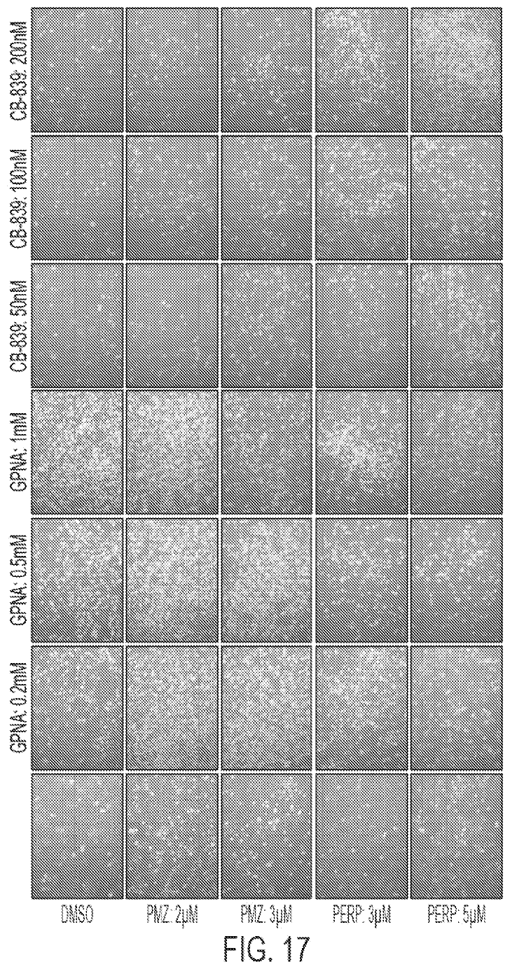

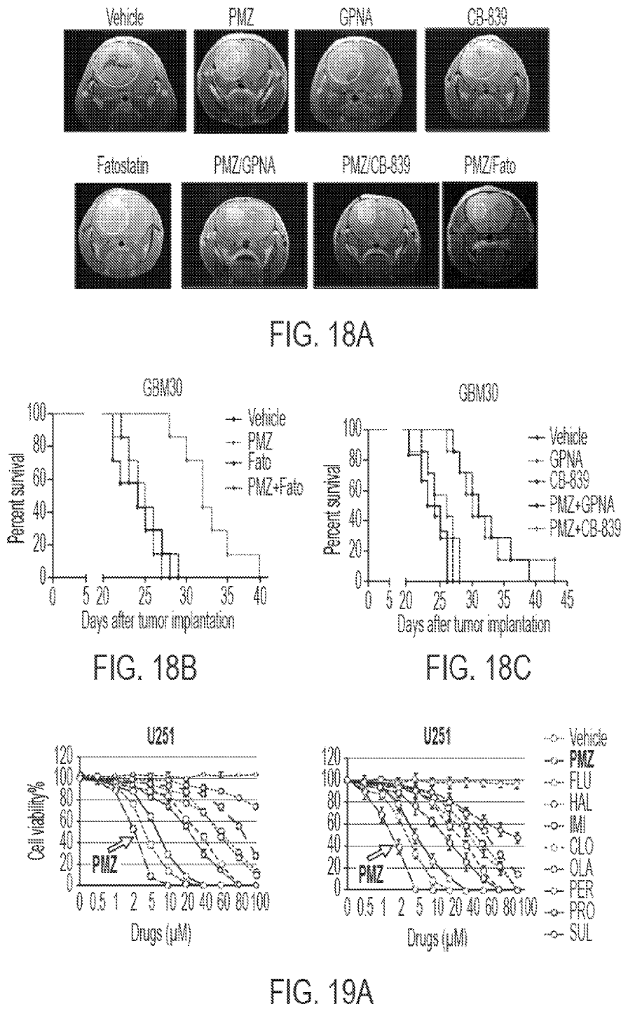

Hematoxylin and eosin (H&E) staining confirmed the dramatically increased number of tumor lesions in the lungs of wild-type SCAP group compared to the GFP and SCAP D428A groups (FIG. 3D and FIG. 3E, FIG. 6B). IHC staining showed that SREBP-1 staining was significantly elevated in lung tumor tissues in the wild-type SCAP group as compared with the GFP group, while this increase was completely abolished by the D428A mutation (FIG. 3D and FIG. 3E). These experiments were repeated with primary GBM30 cells53-55. As in H1299 cells (FIG. 3A), wild-type SCAP expression dramatically increased SREBP-1 and -2 cleavage in GBM30 cells, which was abolished by the D428A mutation (FIG. 3F). These stably transfected GBM cells were implanted into mice brains and tumor growth was examined on Day 12 and 17 by magnetic resonance imaging (MRI). The imaging showed that the tumor volume in the wild-type SCAP group was dramatically greater than in the control GFP group, and the D428A mutation reduced tumor growth to a volume similar to that of the GFP control group (FIG. 3G and FIG. 6C). H&E staining of GBM-bearing mouse brains further showed that the tumor sizes in the different groups on Day 17 were consistent with those detected by MRI imaging (FIG. 3H, left panels and FIG. 15D). IHC staining showed much stronger SREBP-1 staining in the wild-type SCAP group than in the other two groups (FIG. 3H and FIG. 3I). Moreover, the mice implanted with wild-type SCAP expressing cells had significantly shorter survival time than those in the GFP group, and the D428 mutation significantly extended mice survival to levels similar to those in the control group (FIG. 3J). Together, these data from two cancer models demonstrate that disrupting the SCAP-ammonia interaction by mutating the D428 residue significantly suppresses SREBP-1 activation and tumor growth.

SREBPs are master transcription factors that play a critical role in the regulation of lipid metabolism. Interestingly, they are spatially restricted to the ER membrane after synthesis. The mechanisms triggering the exit of SREBPs from the ER for subsequent nuclear translocation and lipogenesis activation have so far remained unclear. In this example, an unprecedented role of ammonia released from glutamine was uncovered as a key activator of SREBP activation and lipid synthesis. Physiological evidence for the connection between glutaminolysis and lipogenesis was also shown by showing the molecular link between GLS expression and SREBP-1 activation in human lung cancer and glioma tissues. Moreover, it was demonstrated that the activation of SREBPs and lipogenesis by glutamine/ammonia in concert with glucose also occurs in melanoma, liver, and breast cancer cells in addition to lung cancer and GBM, suggesting that this is a common mechanism at play in a wide range of cancer types. Altogether, an unanticipated role for ammonia in the regulation SREBP activation and lipid metabolism was revealed.

When glucose and glutamine levels are low, SREBP-1 activation and lipogenesis are accordingly turned down, regardless of oncogenic signaling, which allows tumor cells to preserve the limited amount of nutrients available to maintain basic cellular activity and survival. Hence, tumors function as a well-organized organ that coordinates oncogenic signaling with nutrient availability to dynamically control the activity of anabolic pathways with tumor growth pace. From this perspective, combining oncogenic signaling targeting with nutrient limitation is an effective approach for cancer therapy.

In summary, this example revealed that ammonia released from glutamine acts as a key signaling molecule activating lipid metabolism. Developing technologies or methods that can directly detect the interaction or binding of ammonia to specific targets will be critical to unravel its largely unexplored function. Moreover, there is a need to develop a sensor to directly measure ammonia levels in tissues and the microenvironment to explore its physiological function. An effective cancer therapy is needed to limit ammonia signaling to prevent its stimulation of tumor growth.

Ammonia, released from glutamine, acts in concert with glucose to promote lipogenesis via activation of sterol regulatory element-binding proteins (SREBPs), endoplasmic reticulum (ER)-bound transcription factors that play a central role in lipid metabolism. Ammonia activates the dissociation of glucose-regulated, N-glycosylated SREBP cleavage-activating protein (SCAP) from Insig, an ER-retention protein, via its binding to SCAP aspartate 428 (D428) and serine 326/330 residues, which triggers sequential conformational changes of SCAP, eventually leading to SREBP translocation and lipogenic gene expression. Interestingly, 25-hydroxcycholesterol prevents ammonia to access its binding site on SCAP, thereby blocking binding to SCAP and suppressing SCAP/Insig dissociation. Mutating D428 to alanine (D428A) also prevents ammonia binding to SCAP and ensuing conformational changes, abolishes SREBP-1 activation, and suppresses tumor growth.

SREBPs are synthesized as inactive precursors (˜125 kD) that are retained in the endoplasmic reticulum (ER) membrane and are activated through a tightly controlled ER-Golgi-nucleus translocation process. SREBPs first bind to SREBP-cleavage activating protein (SCAP), which further binds to COPII-coated vesicles that transport the SCAP/SREBP complex from the ER to the Golgi. In the Golgi, SREBPs are sequentially cleaved by site-1 and -2 proteases, which release their N-terminal forms (˜65 kD) that then enter into the nucleus to activate lipogenic gene expression. However, the trafficking of the SCAP/SREBP complex is suppressed by the ER-retention protein, insulin-inducible gene protein (Insig), which includes two isoforms, Insig-1 and -2. Insig binds to SCAP to retain the SCAP/SREBP complex in the ER. It was previously revealed that cholesterol or 25-hydroxycholesterol (25-HC) can bind to SCAP or Insig to enhance their association, which mediates a negative feedback loop to modulate SREBP activation. However, the key step activating the dissociation of SCAP from Insig for subsequent translocation remains unclear.

Herein it was demonstrated that glucose stimulates SREBP activation and lipogenesis by promoting SCAP N-glycosylation and stability. In this example, it was unexpectedly found that when glutamine is lacking, glucose alone is unable to activate SREBPs and lipogenesis despite low cholesterol levels and stable SCAP N-glycosylation. Here, it was revealed that N-glycosylated SCAP requires the stimulation of ammonia released from glutamine to undergo sequential conformational changes in order to dissociate from Insig and promote SREBP translocation and lipogenesis. The binding site of ammonia was identified in the central location of SCAP transmembrane domain, including D428 and serine S326/S330 residues demonstrating that the function of ammonia is prevented by 25-hydroxycholesterol (25-HC), which blocks access to its binding site on SCAP, thereby suppressing SCAP/Insig dissociation and SREBP activation. This example further shows that targeting the key molecular link between glutamine, glucose and lipid metabolism is a strategy for treating malignancies and metabolic syndromes. Thus, inhibiting lipid supplies from internal storage lipid droplets and external lipoproteins by suppressing lysosomal function, a synergistic effect may be realized.

### Example 2: Use of Pimozide In Vitro

The prognosis for patients with glioblastoma (GBM), the most lethal primary brain tumor in adults, has been largely unchanged for the past two decades, with the median survival still remaining at 12-16 months after initial diagnosis, even if the patient has undergone extensive treatment. Thus, there is an urgent need to identify effective new therapies for GBM. By the time of diagnosis, many GBM cells have typically already invaded the surrounding normal brain tissue, making complete surgical resection of the tumor unlikely. Furthermore, the blood-brain barrier (BBB) significantly limits the penetration of many antitumor drugs into GBM tissues. Even if drugs could be efficiently delivered intracranially, GBM cells quickly develop resistance to these drugs, compromising their efficacy and affecting overall outcome. Therefore, GBM is among one of the most difficulty cancers to treat.

Fatty acids (FAs) and cholesterol are two essential lipids for cell growth and proliferation. FAs constitute the hydrophobic tail of phospholipids and cholesterol inserts between phospholipids to regulate membrane fluidity and permeability. It has been shown that GBM cells acquire abundant cholesterol from external sources by upregulating low-density lipoprotein receptor (LDLR)-mediated uptake of LDL, a major cholesterol carrier in the bloodstream. LDL contains abundant cholesterol esters (CEs) that are hydrolyzed in the lysosomes to release free cholesterol and FAs for GBM growth. Lipid droplets (LDs), a hallmark of adipocytes, contain abundant CEs and triacylglycerols (TAGs). It has also been demonstrated that patient derived GBM tissues contain large amounts of LDs, which can also be found in other cancers such as breast, prostate, liver, pancreatic, colon and renal cancers. LD hydrolysis in lysosomes and the release of stored cholesterol and FAs fuel GBM growth. Most recently, it was demonstrated that glutamine-released ammonia (NH4+) activates sterol regulatory element-binding protein 1 (SREBP-1), a key transcription factor that regulates lipogenic gene expression to promote FAs and cholesterol synthesis. Together, it has been demonstrated that GBM cells can aggressively access multiple lipid sources to ensure a sufficient supply of FAs and cholesterol to support their rapid growth. Thus, limiting access of GBM cells to lipid sources, including LDL, LD hydrolysis and de novo synthesis, is an effective approach to target this deadly cancer. However, simultaneously blocking all three lipid sources is very challenging, particularly in a clinically relevant manner.

Recently, many have shown that various antipsychotic drugs exhibit antitumor activities. The proposed antitumor mechanisms for these drugs are very broad, including damaging lysosomes, stimulating autophagy, inhibiting the function of different oncogenes, activating tumor suppressors, and others. Some reported mechanisms are controversial, such as autophagic stimulation despite the induction of lysosomal damage, as it would be expected that autophagic flux would be blocked when lysosomal activity is inhibited. Collectively, the major mechanisms underlying the antitumor effect of these antipsychotic drugs remain unclear. Moreover, these agents only exhibit minor to modest antitumor effects in preclinical animal studies, including in GBM. Why tumor cells lack sensitivity to these drugs and/or how tumor cells become resistance to these drugs have not yet been studied, which limit their repurposing for cancer treatment. Nevertheless, these neuronal-based antipsychotic drugs remain of great interest, particularly for brain tumors, as they can efficiently cross the BBB.

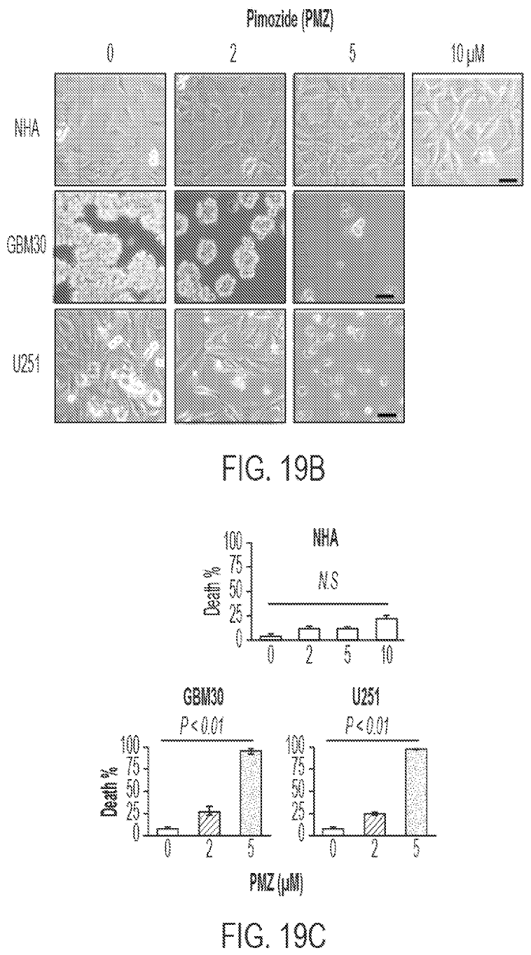

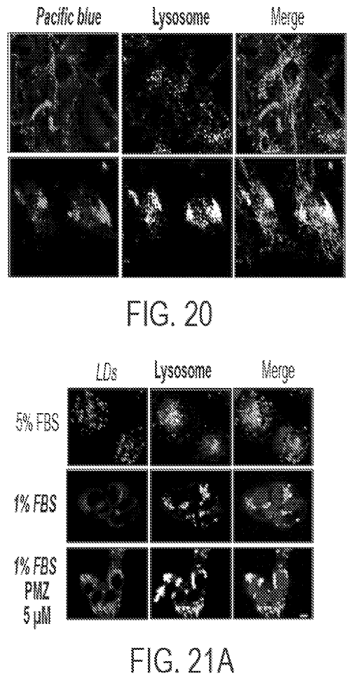

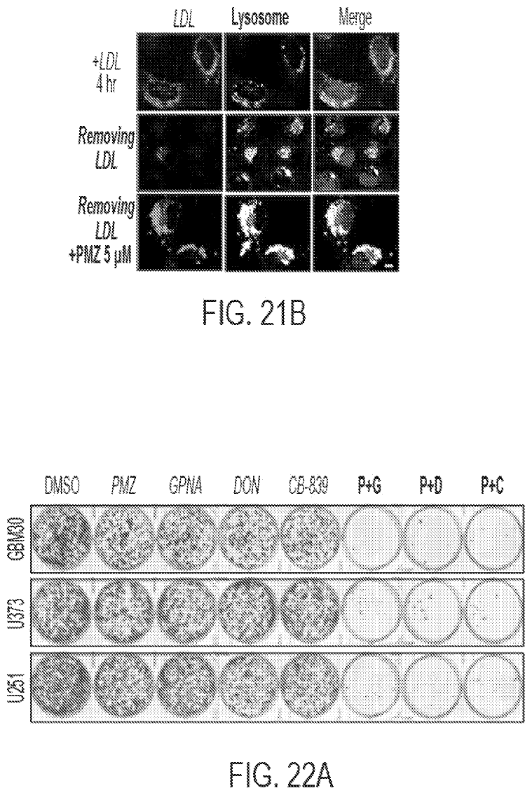

Nine FDA-approved antipsychotic drugs were screened for antitumor effects. Pimozide, which is used to treat schizophrenia, as well as motor and phonic tics associated with Tourette's syndrome, is the most potent drug for killing GBM cells in vitro (FIG. 19A-19C). Pimozide was tested for the treatment of melanoma in a Phase T1 clinical trial (n=30) in the 1980s, while only showed a favorable response in 6 (17%) patients. A recent study reported that pimozide treatment could cause lysosomal membrane permeabilization, leading to the release of proteinase cathepsin into the cytosol, together with overactivation of autophagy causing GBM cell death. In contrast, another study showed that pimozide treatment alone had no significant effect in GBM intracranial models, and only a modest antitumor effect when combined with radiation therapy. These data demonstrate that GBM cells develop resistance to pimozide, but the underlying mechanisms are unknown. The data herein showed that pimozide treatment effectively suppressed the hydrolysis of both LDs and LDL in GBM cells, resulting in their accumulation in the lysosomes (FIG. 21A and FIG. 21B). Supplementation of GBM cells with free FAs and cholesterol markedly rescued pimozide-inhibited cell viability. These results challenge the conclusion by others that pimozide-induced GBM cell death is caused by activation of autophagy and proteinase leaking from the lysosomes as FA/cholesterol addition would not rescue autophagy and proteinase-mediated cell killing. Moreover, the activities of the lysosomal proteinases are mainly dependent on the acidic environment (pH 4.5˜5.0). Once exposed to a neutral environment in the cytosol (pH˜7.0), lysosomal proteinases promptly lose activity.

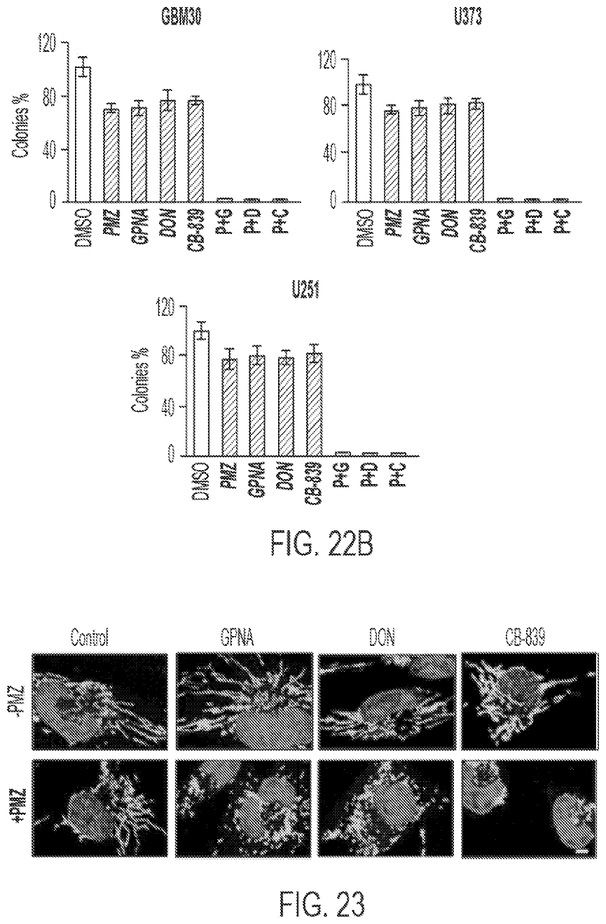

Stable isotope 13C-glutamine tracing experiments were conducted, and it was found that glutamine uptake, glutaminolysis and reductive carboxylation-mediated citrate synthesis derived from glutamine were greatly elevated upon pimozide treatment in vitro. Furthermore, pharmacologically inhibiting either ASCT2, the glutamine transporter, or glutaminase (GLS), which controls the first step of glutaminolysis, to inhibit glutamine uptake or glutaminolysis, respectively, in combination with pimozide resulted in an almost complete eradication of GBM cell-derived colonies, whereas monotherapy with each drug only showed a slight inhibitory effect (FIG. 22A-22B). Additionally, it was observed that the combined treatment induced a striking mitochondrial fragmentation (FIG. 23).

Among nine antipsychotic agents, pimozide is the most potent drug for killing GBM cells in vitro. Nine antipsychotic drugs, including two anti-depressants (imipramine and fluoxetine) were reported to have antitumor effects in vitro or in vivo. Pimozide was found to be the most potent drug to reduce GBM cell viability (FIG. 19A). This drug almost completely killed patient-derived primary GBM30 cells and U251 cells at 5 μM (FIGS. 19B-19C), while it had no toxic effects on normal human astrocytes (NHA), even at doses up to 10 μM.

Fluorescence-labeling pimozide enters into lysosomes in GBM cells. The structure of pimozide indicates that the pKa of the amide residue, located at the center of the molecule, is ˜8.63 (FIG. 19A), which can be quickly protonated in an acidic environment. Lysosomes are the most acidic organelles in cells (pH 4.5˜5.0) and lysosomal hydrolases are fully dependent on this acidic environment for their activity. It was speculated that pimozide enters the lysosomes of GBM cells and is protonated, trapping it in the lysosomal lumen as protonated pimozide, which is positively charged and cannot cross the lysosomal membrane. This process consumes large amounts of lysosomal protons (H+), leading to an increase in lysosomal pH and suppression of the lysosomal hydrolytic function, thereby blocking LD and LDL hydrolysis. However, there is no direct evidence showing that antipsychotic drugs can enter the lysosomes to disrupt the acidic environment.

To test this, Pacific blue, a fluorescent compound, was used to label pimozide (FIG. 20), and cells treated with labeled-pimozide were visualized by confocal microscopy to determine its distribution and examine if it enters the lysosomes. Lysosomes were co-stained with LysoTracker (red). The data showed that pimozide directly enter the lysosomes, while octane-amine linked Pacific blue alone was unable to enter cells and could only bind to the plasma membrane of GBM cells, which excluded any effect of the fluorophore dye on the crossing of the plasma membrane by labeled pimozide (FIG. 20). Thus, the trafficking and subcellular localization of fluorescently labeled pimozide in the cytoplasm of GBM cells faithfully reflect those of the unlabeled pimozide compound.

Pimozide inhibits LD and LDL hydrolysis in GBM cells. To explore how pimozide affects GBM cells, the effects on LD and LDL hydrolysis was examined. Pimozide treatment dramatically suppressed LD and LDL hydrolysis, leading to their accumulation in the lysosomes (FIGS. 21A-21B). Fluorescence imaging also showed that lysosomes became enlarged and swollen and aggregated together after treatment for 24 hr (FIGS. 21A-21B, arrow).

Pharmacological targeting of glutamine metabolism in combination with pimozide nearly eradicates all pre-formed GBM colonies. The data herein further showed that combining pimozide (3 μM) with pharmacological targeting of either the glutamine transporter ASCT2 with gamma-glutamyl-p-nitroanilide (GPNA, 0.5 mM) or the key glutaminolysis enzyme GLS with CB-839 (100 nM), or with the broad antagonist of glutamine metabolism, 6-diazo-5-oxo-I-norleucine (DON, 5 μM) nearly entirely eradicated pre-formed GBM colonies, while each drug alone only had a slight inhibitory effect (FIGS. 22A-22B). These data strongly show that such combinatorial approaches could be promising strategies to target GBM.

Targeting glutamine metabolism in combination with pimozide results in striking mitochondrial damage. It was explored how combining pimozide with the ASCT2 or GLS inhibitors or with DON killed GBM cells. Using fluorescence imaging, each combination resulted in striking mitochondrial fragmentation, while treatments with individual drugs had no significant effect (FIG. 23), strongly showing that these combinations led to mitochondrial damage.

In summary, combining lipogenesis inhibition with lysosome suppression results in simultaneously inhibiting all three lipid sources: de novo lipid synthesis, internal LD storage and external lipoproteins. By markedly reducing lipid levels, a highly effective method killing tumor cells is provided, thereby serving as effective approach for cancer therapy

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

The invention claimed is:

1-82. (canceled)

83. A method of treating cancer in a patient in need thereof, comprising administering the lysosome dysregulating agent in combination with the lipogenesis inhibitor, an ammonia suppressing agent, or a combination thereof.

84. The method of claim 83, comprising administering a lipogenesis inhibitor, wherein the lipogenesis inhibitor comprises a fatty acid synthesis inhibitor.

85. The method of claim 84, wherein the fatty acid synthesis inhibitor comprises fatostatin, FT113, BI99179, BI99179, FASN-IN-4 tosylate, IPI-9119, TVB-3166, TVB-3663, TVB-2640, PF429242, GSK2194069, Pseudoprotodioscin dihyrochloride, Betulin, C75, Lycorine, GSK837149A, ACC1/2-IN-1, ACC1/2-IN-2, JA-ACC, PF-05175157, Firsocostat, MK-4074, CP-610431, 7ACC2, CP-640186, CP-640186, Olumacostat glasaretil, 7ACC1, hACC2-IN-1, CMS-121, Firsocostat, Moiramide B, ND-646, Aceyl-CoA Carboxylase-IN-1, A-908292, Cerulenin, 28UCM, GSK2194069, Orlistat, TOFA, PF-05221304, YTX-465, T-3764518, MF-438, A939572, XEN723, CVT-12012, CAY10566, YTX-7739, MK-8245, GSK1940029, SCD1-IN-1, SCD1 inhibitor-4, eliglustat, ibiglustat, lucrerastat, or a combination thereof.

86. The method of claim 85, wherein the fatty acid synthesis inhibitor comprises fatostatin, TVB-2640, or a combination thereof.

87. The method of claim 83, comprising administering a lipogenesis inhibitor, wherein the lipogenesis inhibitor comprises a SREBP inhibitor.

88. The method of claim 87, wherein the SREBP inhibitor comprises tocotrienol, artesunate, ursolic acid, archazolid B, PF-429242, nelfinavir, cinobufotalin, yridin; 1-(4-bromophenyl)-3-(yridine-3-yl)urea, firsocostate, YTX-7739, TVB-2640, PF-05221304; ND646; PF-05175157, CP 640186, NB-598, terbinafine, or a combination thereof.

89. The method of claim 87, wherein the SREBP inhibitor comprises an HMG-CoA inhibitor.

90. The method of claim 89, wherein the HMG-CoA inhibitor comprises simvastatin.

91. The method of claim 83, wherein the lysosome dysregulating agent comprises an antipsychotic, an antimalarial, or a combination thereof.

92. The method of claim 91, wherein the lysosome dysregulating agent comprises pimozide, chloroquine, hydroxychloroquine, or a combination thereof.

93. The method of claim 83, comprising administering an ammonia suppressing agent, wherein the ammonia suppressing agent comprises a glutaminase inhibitor.

94. The method of claim 93, wherein the glutaminase inhibitor comprises 6-diazo-5-oxonorleucine, bis-2-(5-phenylacetamido-1,3,4-thiadiazol-2-yl) ethyl sulphide, 5-(3-bromo-4-(dimethylamino)phenyl)-2,2-dimethyl-2,3,5,6-tetrahydrobenzo[a]phenanth ridin-4(1H)-one, telaglenastat, ethyl 2-(2-Amino-4-methylpentanamido)-6-diazo-5-oxonorleucine, IPN60090, GK921; UPGL00004; BPTES; JHU395; JHU-083, or a combination thereof.

95. The method of claim 94, wherein the glutaminase inhibitor comprises telaglenastat.

96. The method of claim 83, comprising administering an ammonia suppressing agent, wherein the ammonia suppressing agent comprises an ACST2 inhibitor.

97. The method of claim 96, wherein the ACST2 inhibitor comprises GPNA.

98. The method of claim 83, wherein the cancer comprises lung cancer, breast cancer, glioblastoma, prostate cancer, liver cancer, pancreatic cancer, colon cancer, or renal cancer.

99. The method of claim 83, wherein the cancer comprises glioblastoma.

100. The method of claim 83, comprising administering pimozide, chloroquine, hydroxychloroquine, or a combination thereof, and TVB-2640, telaglenastat, simvastatin, fatostatin, GPNA, or a combination thereof.
